(19) United States
(12) Patent Application Publication
(10) Pub. No.: US 2009/0293142 A1
(43) Pub. Date: Nov. 26, 2009

(54) Methods for increasing shoot-to-root ratio, seed production and resistance to diseases

(75) Inventors: Roitsch Thomas, Wurzburg (DE); Maria de la Cruz Gonzalez Garcia, Sevilla (ES)

Correspondence Address: MDIP LLC, POST OFFICE BOX 2630, MONTGOMERY VILLAGE, MD, 20886-2630, US

(73) Assignee: JULIUS-MAXIMILIANS-UNIVERSITAT WURZBURG, Wurzburg (DE)

(21) Appl. No.: 12/281,508

(22) PCT Filed: Mar. 7, 2007
(86) PCT No.: PCT/EP07/01964

§ 371 Date: Dec. 12, 2008

(30) Foreign Application Priority Data
Mar. 7, 2006 (EP) 06004640.6

(51) Int. Cl. C12N 15/82; A01H 1/06; A01H 5/00; C07H 21/04; C07K 14/415; C12N 15/63; C12N 5/10; A01N 1/00; G01N 33/68

(52) U.S. Cl. 800/276; 800/279; 800/278; 800/298; 800/301; 536/23.6; 536/24.5; 530/350; 435/320.1; 435/419; 435/1.1; 435/468; 436/86

(57) ABSTRACT

The present invention is related to a method for increasing shoot-to-root ratio of a plant comprising the step of
inhibiting the activity of an invertase in the root tissue of said plant.

The present invention is related to methods for increasing shoot-to-root ratio, seed production and resistance to diseases.

Furthermore the present invention is related to new invertases and the use thereof.

Shoot-to-root ratio, seed production and pathogen resistance in plants are particularly linked to carbohydrate metabolism. More specifically, in higher plants, growth and metabolism of sink tissues is sustained by the carbohydrates synthesized in source leaves and transported, mainly in the form of sucrose, thorough the phloem into the sink tissues. Source-sink relationships have been shown to change with plant growth and development and in response to different biotic and abiotic stresses. In concrete, the acquisition of fixed carbon from the shoot by the roots seems to be determined both by the availability of, and the need for, assimilates in the shoot and root respectively (Farrar and Jones, 2000). Use of sucrose in the sink tissues requires cleavage of the glycosidic bond, catalysed both by sucrose synthase and invertases. Sucrose synthase cleaves sucrose into UDP-glucose and fructose, whereas invertases hydrolyse sucrose into the hexose monomers. Three types of invertase isoenzymes are distinguished based solubility, sub-cellular localization, pH optima and isoelectric point (Roitsch and González, 2004). Between them, cell-wall bound invertases have been shown to play a crucial function in carbohydrate partitioning and supply of photoassimilates to sink tissues (Tang et al., 1999; Goetz et al., 2001; Weschke et al., 2003). Cleavage of sucrose at the site of phloem unloading and transport of the generated hexoses into the sink cells, through the concerted action of cell-wall invertases and hexose transporters, generates differences in osmotic pressure that drive the transport of sucrose in the phloem. An apoplasmic unloading of sucrose is not only characteristic of symplasmically isolated tissues but also of actively growing tissues, like maize primary root tips, where the demand of photoassimilates cannot be satisfied solely by the symplasmic unloading (Bret-Harte and Silk, 1994). The high invertase activity reported in root tips and site of emergency of secondary roots supports a role of cell-wall invertase in active growth of this sink tissue (Eschrich, 1980). In Arabidopsis thaliana, the expression pattern of cell-wall and vacuolar invertases in the root during development and in response to different culture conditions suggests that cell wall invertase is involved in sucrose partitioning in conditions with a high assimilated demands in this tissue. In mature roots, however, cell wall invertase expression is not detected and vacuolar invertase expression would be responsible for sucrose incoming (Tymowska-Lalanne and Kreis, 1998). Phloem unloading in mature roots would then fit to the model proposed by Sturm et al. (1995), where the driving force for sucrose unload results from the cleavage of the sugar by sucrose synthase and vacuolar invertase in the cytosol. In carrot tap roots, the effect of antisense inhibition of vacuolar and cell wall invertases on plant phenotype suggests an important role in sucrose partitioning (Tang et al., 1999). In addition, vacuolar invertase may be a key regulator of cell expansion, due to the doubled osmotic potential generated by sucrose cleavage in the vacuole.

Given this highly complex interaction of elements of plant carbohydrate metabolism, the problem underlying the present invention is to identify methods for increasing shoot-to-root ratio, seed production and resistance to diseases in plants.

The problem underlying the present invention is solved in a first aspect by a method for increasing shoot-to-root ratio of a plant comprising the step of
inhibiting the activity of an invertase in the root tissue of said plant.

The problem underlying the present invention is solved in a second aspect by a method for increasing seed production of a plant comprising the step of
inhibiting the activity of an invertase in the root tissue of said plant.

The problem underlying the present invention is solved in a third aspect by a method for increasing resistance to a disease of a plant comprising the step of
inhibiting the activity of an invertase in the root tissue of said plant.

In an embodiment according to the first, the second and the third aspect of the present invention the activity of the invertase is inhibited either by
(a) a knock-down of the invertase or
(b) knock-out of the invertase, or
(c) an inhibitor to the invertase.

It is to be noted that if not indicated to the contrary, any of the following embodiments is an embodiment of the first, the second and the third aspect of the present invention.

In an embodiment the inhibitor is active in the root tissue of the plant.

In an embodiment the inhibitor is a polypeptide.

In an embodiment the inhibitor is encoded by a nucleic acid.

In an embodiment the nucleic acid is under the control of a transcription element and/or a translation element, whereby such transcription element and/or such translation element allows for the specific transcription and/or translation of the nucleic acid in root tissue.

In an embodiment the transcription element is a promoter, preferably a root specific promoter.

In an embodiment the promoter is an inducible promoter.

In an embodiment the promoter is selected from the group comprising promoter pyk20, promoter T80-cryptic, and promoter WRKY6.

In an embodiment the promoter is promoter T80-cryptic.

In an embodiment the inhibitor is selected from the group comprising tobacco invertase inhibitors and Arabidopsis invertase inhibitors, whereby, preferably, the tobacco invertase inhibitors are selected from the group comprising NT-CIF1, Y12805; Nt-VIF, AY145781, and/or, preferably, the Arabidopsis invertase inhibitors are selected from the group comprising AtCNVIF1, At1g47960; AtCNVIF2, At5g64620, and AtCNVIF3, At3g17130.

In an embodiment the knock-down is caused by post-transcriptional gene silencing and/or co-suppression.

In an embodiment the invertase is an invertase selected from the group comprising a soluble invertase, a vacuolar invertase, a neutral/alkaline invertase and a cytoplasmatic invertase.

In an embodiment the invertase is an invertase selected from the group comprising a cell wall bound invertase, and an extracellular, apoplasmic but not cell wall bound invertase, whereby preferably the invertase is a cell wall bound invertase.

In an embodiment the invertase is an invertase having an amino acid sequence, whereby the amino acid sequence is encoded by a nucleic acid which is selected from the group of nucleic acid sequences comprising nucleic acid sequences SEQ.ID.No. 23 to 36.

In an embodiment the invertase is an invertase having an amino acid sequence, whereby the amino acid sequence is encoded by a nucleic acid which is selected from the group of nucleic acid sequences comprising nucleic acid sequences SEQ.ID.No. 1 to 22.

In an embodiment the disease of the plant is a disease involving or affecting the root tissue

In an embodiment the disease of the plant is transferred or caused by a pathogen.

In an embodiment the pathogen is selected from the group comprising Plasmodiophora brassicacae, Verticillium and nematodes, whereby the nematode preferably is Heterodera schachtii Schm.

In an embodiment the disease is selected from the group comprising diseases which are caused by or associated with an organism selected from the group comprising Pythium aphanidermatum, Pythium ultimum, Phytophthora syringae P. undulata, Oxysporum f. sp. radicis-lycopersici, Meloidogyne hapla, Phytophtora quercina and Rhizoctonia solani Kühn.

In an embodiment the plant is a member of the family of Brassicacae.

In an embodiment the plant is selected from the group comprising rapeseed, cabbage and china cabbage.

The problem underlying the present invention is solved in a fourth aspect by a nucleic acid molecule, preferably coding for an invertase, having a nucleic acid sequence, whereby the nucleic acid sequence is selected from the group of nucleic acid sequences SEQ.ID.No. 1 to 36, or a nucleic acid essentially complementary thereto.

The problem underlying the present invention is solved in a fifth aspect by a nucleic acid molecule which hybridizes, preferably under stringent conditions, to the nucleic acid sequence according to the fourth aspect of the present invention.

The problem underlying the present invention is solved in a sixth aspect by a nucleic acid molecule which, but for the degeneracy of the genetic code, would hybridize, preferably under stringent conditions, to the nucleic acid according to the fourth or the fifth aspect of the present invention.

The problem underlying the present invention is solved in a seventh aspect by a polypeptide, preferably an invertase, encoded by a nucleic acid molecule according to any of the fourth, the fifth and the sixth aspect of the present invention.

The problem underlying the present invention is solved in a eighth aspect by a vector comprising a nucleic acid molecule according to any of the fourth, the fifth and the sixth aspect of the present invention.

In an embodiment the vector is a plant vector, more preferable a plant expression vector.

In an embodiment the vector comprises a root specific promoter.

The problem underlying the present invention is solved in a ninth aspect by a cell, preferably a plant cell, comprising nucleic acid molecule according to any of the fourth, the fifth and the sixth aspect of the present invention and/or a vector according to the eighth aspect of the present invention.

The problem underlying the present invention is solved in a tenth aspect by a tissue and/or an organ comprising a nucleic acid molecule according to any of the fourth, the fifth and the sixth aspect of the present invention and/or a vector according to the eighth aspect of the present invention and/or a cell according to the ninth aspect of the present invention.

In an embodiment of the tenth aspect the tissue is a root tissue and/or the organ is a root.

The problem underlying the present invention is solved in a eleventh aspect by an organism, preferably a plant, comprising a nucleic acid molecule according to any of the fourth, the fifth and the sixth aspect of the present invention and/or a vector according to the eight aspect of the present invention and/or a cell according to the ninth aspect of the present invention.

The problem underlying the present invention is solved in a twelfth aspect by the use of a nucleic acid construct for the modification of the genome of a plant, whereby the construct comprises
(a) a root specific promoter; and
(b) a nucleic acid coding for an invertase inhibitor,
   wherein the promoter and the nucleic acid coding for the invertase are operably linked to each other.

The problem underlying the present invention is solved in a thirteenth aspect by the use of a nucleic acid construct for inhibiting the activity or presence of an invertase, preferably an invertase in root and/or root tissue, whereby the construct comprises
(c) a root specific promoter; and
(d) a nucleic acid coding for an invertase inhibitor,
   wherein the promoter and the nucleic acid coding for the invertase are operably linked to each other.

The problem underlying the present invention is solved in a fourteenth aspect by the use of a nucleic acid construct for increasing shoot-to-root ratio, seed production and/or resistance to disease of a plant, whereby the construct comprises
(e) a root specific promoter; and
(f) a nucleic acid coding for an invertase inhibitor,
   wherein the promoter and the nucleic acid coding for the invertase are operably linked to each other.

The problem underlying the present invention is solved in a fifteenth aspect by the use of a nucleic acid construct for the manufacture of a medicament for the treatment of a plant disease, whereby the construct comprises
(g) a root specific promoter; and
(h) a nucleic acid coding for an invertase inhibitor,
   wherein the promoter and the nucleic acid coding for the invertase are operably linked to each other.

In an embodiment of the fifteenth aspect of the present invention the medicament is for gene therapy of a plant.

In an embodiment of the fifteenth aspect of the present invention the plant is a plant cell or a plant tissue, preferably prior to regeneration to a mature plant.

The problem underlying the present invention is solved in a sixteenth aspect by the use of a nucleic acid construct for the generation of a transgenic plant, whereby the transgenic plant preferably shows one or more of an increase in shoot-to-root ratio, increase in seed production and increase in resistance to pathogens and/or diseases.

The problem underlying the present invention is solved in a seventeenth aspect by the use of a polypeptide according to the seventh aspect of the present invention as a target molecule.

In an embodiment of the seventeenth aspect the polypeptide is a target molecule for an inhibitor in vitro and/or in vivo.

In an embodiment of the seventeenth aspect the target molecule is a target molecule in the root tissue of a plant.

In an embodiment of the seventeenth aspect of the present invention the target molecule is used in s screening method for the identification of a plant protection agent.

In an embodiment of the seventeenth aspect of the present invention the target molecule is used in s screening method for the identification of a plant growth promoter.

The present inventors have surprisingly found that invertases and more specifically root invertase activity is a suitable target for solving the problems underlying the present invention. More specifically, the present inventor has found that the inhibition of invertase activity and more particularly invertase activity in root and root tissue, respectively, is a suitable means for increasing, in plants, the shoot-to-root ratio, the seed production and resistance to disease in general and root related or root associated diseases in particular. Contrary to what may have been expected by the one skilled in the art, a reduced invertase activity in the root produced a slight increase rather than a decrease in root fresh weight. The most pronounced effect of inhibiting invertase activity in root was, however, observed in the shoots, namely an increase in biomass with respect to control plants and an associated increase in shoot-to-root ratio. The increased growth of the areal part of the plant resulted in an increased production of shoots and silique number and, as a consequence, an increased seed yield in this kind of plant. In other words, root-specific compression or inhibition of an invertase is suitable to trigger these effects. Insofar, the technical teaching of the present invention is to decrease the activity of an invertase, preferably an invertase in the root and root tissue, respectively, of a plant for which the above mentioned effects are desirable. In principle, there is a number of means and ways available to decrease invertase activity in plant tissue and more specifically in root tissue. One such means is a knock-down of the mRNA coding for such invertase, another one is the use of an inhibitor to the invertase whereby such inhibitor is administered to the plant to be treated, preferably by means of genetic engineering.

In a preferred embodiment of the present invention, the inhibition of the invertase activity occurs at the post-translational level. This provides for the option that the overall invertase activity is factually decreased or inhibited which is in contrast to the phenomenon frequently observed with knock-down of a single invertase coding gene, where the plants typically react by up-regulating a different invertase gene.

The term knock-down as used herein preferably also comprises the knock-out of the invertase activity. In connection with the present invention, a knock-down or knock-out thus goes along with a decreased activity of the invertase which is knocked-down or knocked-out. Such decrease in activity of an invertase is typically at least a reduction in five, more preferably 10, more preferably 20 or more percent of activity compared to the non-knocked-down activity. It will be acknowledged by the ones skilled in the art that a knock-out or knock-down of the invertase can also be effected by a knock-out or a knock-down of an activator or other factor providing for the activity and/or expression of the invertase.

The activity of an invertase is preferably defined as the hydrolysis of sucrose into the hexose monomers. Respective assay systems for measuring the activity of invertases are known to the ones skilled in the art, and, for example, described in Roitsch et al. (1995) (Roitsch T., Bittner M., Godt D. E. (1995). Induction of apoplastic invertase of Chenopodium rubrum by D-glucose and a glucose analog and tissue-specific expression suggest a role in sink-source regulation. Plant Physiol. 108, 285-294).

Basically, the invertase assay is performed in an embodiment as follows. A soluble protein extract is obtained by homogenisation of the tissue in a homogenisation buffer. An insoluble protein fraction is obtained by shaking the insoluble pellet in high salt buffer overnight. After dialysis of these fractions, vacuolar, neutral and extracellular invertase activity in the corresponding fractions are measured by determining the amount of glucose released in a reaction with sucrose as a substrate and at the corresponding pH by use of a buffer. Glucose released is measured by use of a coupled assay with glucose oxidase and peroxidase enzymatic activities. The concentration of glucose released in the reaction is calculated from the OD value by use of a calibration curve. In all cases, control reactions using the same volume of water instead of sucrose in the reaction mixture are performed. Invertase activity for each sample is preferably determined in triplicate and normalised to the concentration of protein in the assay determined by the Bradford method (1976) with the Bio Rad kit.

A preferred way to knock down an invertase which preferably means degrading the mRNA coding for the invertase, is post-transcriptional gene silencing in plants such as, for example, by an antisense construct. This kind of technology is, among others, described in Mol. J. N. et al. (Mol. J. N., van der Krol A. R., van Tunen A. J., van Blokland R., de Lange P. and Stuitje A. R. (1990). Regulation of plant gene expression by antisense RNA. FEBS Lett. 268, 427-430).

Another possibility to knock down an invertase is by using RNA-interference technology as, e.g., described in Kusaba M. (Kusaba M. (2004). RNA interference in crop plants. Curr. Opin. Biotechnol. 15, 139-143) or in Matzke M. A. et al (Matzke M. A., Matzke A. J., Pruss G. J. and Vance V. B. (2001). RNA-based silencing strategies in plants. Curr. Opin. Genet. Dev. 11, 221-227).

A still other possibility to knock down an invertase is by using co-suppression as, e.g., described by Vaucheret H. (Vaucheret H., Beclin C. and Fagard. Post-transcriptional gene silencing in plants. J. Cell Sci. 114, 3083-3091), or by Vance V. (Vance V. and Vaucheret H. (2001). RNA Silencing in Plants-Defense and Counterdefense. Science 292, 2277-2280).

A second approach for inhibiting or decreasing the activity of an invertase is by an inhibitor to such invertase. Such inhibitors to invertases are, in principle, known to the one skilled in the art. It is within the present invention that preferably any invertase inhibitor can be used in connection with the present invention, more preferably any invertase inhibitor under the proviso that the respective inhibitor is inhibiting the activity of an invertase, most preferably an invertase expressed or active in the root tissue of a plant, whereby route tissue as used herein preferably means both intercellular to the root tissue as well as extracellular to the root tissue.

In a preferred embodiment, the inhibitor of an/the invertase is a polypeptide. As preferably used herein, a polypeptide is a polymer comprising at least two amino acids which are linked to each other by a peptide bond. More preferably, the polypeptide comprises 6, 10, 25 or more amino acids, whereby the upper range is preferably 50, 100, 200 and 500 amino acids. In connection with the present invention, the term polypeptide and protein are used in a synonymous manner. Preferably, the size of invertase inhibitors is approximately 500 nucleotides and 166 to 192 amino acids, respectively, according to a comparison done in Rausch and Greiner (2004) (Rausch T. and Greiner S. (2004). Plant protein inhibitors of invertases. Biochim. Biophys. Acta 1696, 253-261), and the molecular weight is approximately 18 kDa. The protein sequence is not well conserved, although a stronger sequence conservation is observed in the N-terminal part than in the C-terminals, and extracellular and vacuolar invertase inhibitors from the same species share only 47% identity at the sequence level, with four cysteines at positions conserved in all invertase inhibitors described so far.

Further invertase inhibitors which can be used in an embodiment of the present invention are described in Greiner S. et al. (Greiner S., Krausgrill S, and Rausch T. (1998). Cloning of a tobacco apoplasmic invertase inhibitor. Proof of function of the recombinant protein and expression analysis during plant development. Plant Physiol. 116, 733-742); Greiner S. et al. (Greiner S., Rausch T., Sonnewald U. and Herbers K. (1999). Ectopic expression of a tobacco invertase inhibitor homolog prevents cold-induced sweetening of potato tubers. Nature Biotech. 17, 708-711), Krausgrill S. et al. (Krausgrill S., Sander A., Greiner S., Weil M. and Rausch T. (1996). Regulation of cell wall invertase by a proteinaceous inhibitor. J. Exp. Bot. 47, 1193-1198), Krausgrill S. et al. (Krausgrill S., Greiner S., Koster U., Vogel R. and Rausch T. (1998). In transformed tobacco cells the apoplasmic invertase inhibitor operates as a regulatory switch of cell wall invertase. Plant J. 13, 275-280), Link M. et al. (Link M., Rausch T. and Greiner S. (2004). In Arabidopsis thaliana, the invertase inhibitors AtC/VIF1 and 2 exhibit distinct target enzyme specificities and expression profiles. FEBS Lett. 573, 105-109), Rausch T. and Greiner S. (2004). Plant protein inhibitors of invertases. Biochim. Biophys. Acta 1696, 253-261), Sander A. et al. (Sander A., Krausgrill S., Greiner S., Weil M. and Rausch T. (1996). Sucrose protects cell wall invertase but not vacuolar invertase against proteinaceous inhibitors. FEBS Lett. 385, 171-175), Weil M. et al. (Weil M., Krausgrill S., Schuster A. and Rausch T. (1994). A 17-kDa Nicotiana tabacum cell-wall peptide acts as in-vitro inhibitor of the cell-wall isoform of acid invertase. Planta 193, 438-445) and Wolf S. et al (Wolf S., Grsic-Rausch S., Rausch T. and Greiner S. (2003). Identification of pollen-expressed pectin methylesterase inhibitors in Arabidopsis. FEBS Lett. 555, 551-555).

Further invertase inhibitors which are useful in the practice of the present invention, are described in Gerrits, N. et al. (Gerrits, N., Turk, S., van Dun, K., Hulleman, S., Visser, R., Weisbeek, P., Smeekens, S. Sucrose Metabolism in Plastids. Plant Physiol. 2001 February; 125(2):926-934), Koch, K. (Koch, K. Sucrose metabolism: regulatory mechanisms and pivotal roles in sugar sensing and plant development. Curr Opin Plant Biol. 2004 June; 7(3):235-246.); Plant invertase inhibitors: Expression in cell culture and during plant development AU: Greiner,-Steffen [Author]; Koster,-Ulrike [Author]; Lauer,-Katja [Author]; Rosenkranz,-Heiko [Author]; Vogel,-Rolf [Author]; Rausch,-Thomas [Reprint-author] SO: Australian-Journal-of-Plant-Physiology. 2000; 27 (8-9): 807-814, Hothorn, M. et al. (Hothorn, M., D'Angelo, I., Marquez, J. A., Greiner, S., Scheffzek, K. The invertase inhibitor Nt-CIF from tobacco: a highly thermostable four-helix Bundle with an unusual N-terminal extension. J Mol. Biol. 2004 January 23; 335(4):987-995), Hothorn, M. et al. (Hothorn, M., Wolf, S., Aloy, P., Greiner, S., Scheffzek, K. Structural insights into the target specificity of plant invertase and pectin methylesterase inhibitory proteins. Plant Cell. 2004 December; 16(12):3437-3447), Sonnewald, U. et al. (Sonnewald, U., Brauer, M., von Schaewen, A., Stitt, M., Willmitzer, L. Transgenic tobacco plants expressing yeast-derived invertase in either the cytosol, vacuole or apoplast: a powerful tool for studying sucrose metabolism and sink/source interactions. Plant J. 1991 July; 1(1):95-106), Bate N J et al. (Bate N J, Niu X, Wang Y, Reimann K S, Helentjaris T G. An invertase inhibitor from maize localizes to the embryo surrounding region during early kernel development. Plant Physiol. 2004 January; 134(1):246-54), Scognamiglio M A et al. (Scognamiglio M A, Ciardiello M A, Tamburrini M, Carratore V, Rausch T, Camardella L. The plant invertase inhibitor shares structural properties and disulfide bridges arrangement with the pectin methylesterase inhibitor. J Protein Chem. 2003 May; 22(4):363-9), Sayago J E et al. (Sayago J E, Vattuone M A, Sampietro A R, Isla M I. An invertase inhibitory protein from Pteris deflexa link fronds. J Enzyme Inhib. 2001 December; 16(6):517-25), Sayago J E et al. (: Sayago J E, Vattuone M A, Sampietro A R, Isla M I. Proteinaceous inhibitor versus fructose as modulators of Pteris deflexa invertase activity. J Enzyme Inhib Med. Chem. 2002 April; 17(2):123-30), Ordonez R M et al. (Ordonez R M, Isla M I, Vattuone M A, Sampietro A R. Invertase proteinaceous inhibitor of Cyphomandra betacea Sendt fruits. J Enzyme Inhib. 2000; 15(6):583-96), and Cheng S H et al. (Cheng S H, Liu J, Song B T, Xie C H. Related Articles, [Cloning of potato invertase inhibitor St-inh cDNA and its expression in E. coli and functional analysis] Shi Yan Sheng Wu Xue Bao. 2004 August; 37(4):269-75. Chinese).

In a preferred embodiment, the inhibitor is encoded by a nucleic acid. It will be acknowledged by the ones skilled in the art that, based on the amino acid sequence of an inhibitor of an invertase, in principle the coding sequence for the inhibitor can be perceived by the one skilled in the art. Due to the generacy of the genetic code, however, quite a number of different sequences is possible. In view of this the one skilled in the art will take into consideration the preferred codon usage of the plant or plant species into which the inhibitor of the invertase is to be introduced, preferably to be introduced by means of transferring the nucleic acid coding for said inhibitor. More preferably, the nucleic acid coding for such inhibitor will be a nucleic acid coding for the inhibitor, whereby such inhibitor is preferably isolated from bacterial, fungal and plant sources.

Among the variety of known invertase inhibitors which are, in principle, suitable for use in the present invention, the tobacco cell wall invertase inhibitor as described by Greiner et al. 1998, a cell wall invertase inhibitor (At5g46940), also referred to herein as AtCNVIF2 and a vacuolar invertase inhibitor At1g47960, also referred to herein as AtC/VIF1 both from Arabidopsis, are particularly suitable for the practice of the present invention. It is known that AtCNVIF1 (At1g47960) specifically inhibits vacuolar invertase activity, whereas AtCNVIF2 (At5g46940) inhibits both, i.e. vacuolar invertase activity as well as cell wall invertase activity, although it has a ten fold higher affinity for vacuolar than for cell wall invertase (Link et al., 2004). A further suitable invertase inhibitor is Nt-inhl as described in international patent application WO 98/04722.

Apart from the afore-mentioned specific invertase inhibitors, further invertase inhibitors are available and in principle suitable for the practice of the present invention. More specifically, such invertase inhibitors are those derived from the 14 genes of the Arabidopsis thaliana genome with sequence identity to tobacco cell wall and vacuolar invertase inhibitors. From these, two genes, At1g47960 and At3g17130, group with the tobacco invertase inhibitors, and two more, At1g48020 and At3g17220, with pectin methylesterase inhibitors. At2g31430, At3g55680, At5g64620, At3g12880 and At5g50070 do not group with any of them in a phylogenetic tree, whereas At5g46940/70/60/80 form a subgroup linked on chromosome 5 (Rausch and Greiner, 2004, supra).

Invertase inhibitor activity is determined in a preferred embodiment by the use of purified protein fractions for both the invertase inhibitor and the corresponding invertase activity. For the determination of invertase inhibitor activity, the invertase and invertase inhibitor preparations are mixed and pre-incubated at 37° C. for 1 hour. After this pre-incubation, sucrose is added to a concentration of 20 mM and the reaction is incubated at 26° C. during 30 minutes. The amount of glucose released in the assay is determined enzymatically as described in Weil M. et al. (Weil et al., 1994; Weil M., Krausgrill S., Schuster A. and Rausch T. (1994). A 17-kDa Nicotiana tabacum cell-wall peptide acts as in-vitro inhibitor of the cell-wall isoform of acid invertase. Planta 193, 438-445), or Greiner S. et al. (Greiner S., Krausgrill S, and Rausch T. (1998). Cloning of a tobacco apoplasmic invertase inhibitor. Proof of function of the recombinant protein and expression analysis during plant development. Plant Physiol. 116, 733-742), or Link M. et al. (Link M., Rausch T. and Greiner S. (2004). In Arabidopsis thaliana, the invertase inhibitors AtCNVIF1 and 2 exhibit distinct target enzyme specificities and expression profiles. FEBS Lett. 573, 105-109). In the determination of invertase inhibitor activity complications may arise from the fact that not purified extract but crude extracts, containing in addition invertase enzymatic activities are used. Additional complication may reside in the fact that the complex formed between the invertase and the inhibitor can, in principle, dissociate during the preparation of the extracts. For these reasons, a “mixed-extract” assay is preferably used in which an aliquot of the root extract of a transgenic plant, therefore expressing the invertase inhibitor, is mixed with an aliquot of a leaf extract of a wild-type plant. The mix, done in the appropriate pH buffer, is incubated 30 min at 37° C. for the formation of the complex between the invertase and the invertase inhibitor. After the pre-incubation, sucrose is added at a final concentration of 5 mM and the reaction incubated for 30 min at 26° C. The reaction is stopped in ice and the glucose released determined by GOD test and compared to the added value of the independent extracts incubated separately. Whereas control roots give values even higher than the corresponding added values, a reduction of up to 50% of the added values is obtained for transgenic roots.

It is obvious for the one skilled in the art that the expression of the nucleic acid coding for the inhibitor has to be controlled by control elements. Preferably, such control elements are active at the transcription level and/or the translation level. In connection with the present invention it has been found to be particularly suitable to have a transcriptionally active element such a promoter for controlling the availability and activity, respectively, of the inhibitor in a cell and tissue, respectively, so as to inhibit invertase activity. In order to provide for the tissue specificity of invertase inhibition as subject to a preferred embodiment of the various aspects of the present invention, namely root tissue specificity, and thus to increase shoot-to-root ratio, seed production and resistance to a disease, the promoter is a root-specific promoter. Such promoter is preferably operately linked to the nucleic acid coding for the inhibitor.

Promoters suitable for such purpose are, in principle, known to the one skilled in the art. More preferred promoters are the following: the promoter pPyk10 which is a promoter of an Arabidopsis mirosinase (Nitz et al., 2001 and also described in international patent application WO 01/44454 and German patent application DE 19960843) and the cryptic promoter also referred to herein as T80-cryptic promoter as described in European patent application EP 1 196 581 and Mollier et al. 2000, Plant-Cell-Reports, 19, 1076-1083, which are both Arabidopsis root-specific promoters. Other promoters which, in principle, are suitable in the practice of the present invention are those root-specific promoters described in international patent applications WO 02/040687 which describes different tissue specific promoters isolated from sugar beets, especially two root specific promoters 2-1-48 and 2-1-36 and those described in WO 00/77187. The promoters described in WO 00/77187 are isolated from tomato and tobacco which is both tapetum specific and tobacco specific.

Additionally, inducible promoters can be used such as those which are inducible by steroids as described, for example, by Zuo et al. (Zuo et al., 2002).

It is within the present invention, that the invertase is actually any invertase activity which is preferably present in roots and/or root tissue and which may be targeted by the methods disclosed herein, namely by knock-down and/or an inhibitor activity. It will be acknowledged by the one skilled in the art that the specificity of the inhibitor to an invertase has to be given at least to the extent that the inhibitor is suitable to inhibit the or some of the activity of an invertase activity in roots and root tissue, respectively. Preferred invertases which can thus be targeted are those described herein, and more specifically the invertase having a nucleic acid sequence according to SEQ. ID. NO. 15 or SEQ. ID. NO. 26. The following table represents varies invertases from Arabidopsis which are suitable in the practice of the present invention as targets as well as the tissue/organ where they are expressed.

| | | | | | Small | | | | | | |
|---|---|---|---|---|---|---|---|---|---|---|---|
| | | | | | flower | Big flower | | | | Siliques | |
| Invertase | Gene locus | Leaf | Stem | Root | bud | bud | Flower | Anthers | Pistil | young | Seedling |
| AtcwINV 1 | At3g13790 | +++ | + | ++++ | ++ | ++ | + | − | + | + | +++ |
| AtcwINV 2 | At3g52600 | − | − | − | − | + | ++ | ++++ | + | − | − |
| AtcwINV 4 | At2g36190 | − | − | − | −/+ | + | ++ | +++ | + | − | − |
| AtcwINV 5 | At3g13784 | ++ | + | ++ | − | + | + | +++ | + | + | ++++ |
| Atβfruct 3 | | ++ | + | ++++ | − | + | ++ | ++ | + | + | |
| Atβfruct 4 | | +++ | +++ | ++ | + | ++ | +++ | +++ | ++ | ++ | |

In this table the level of mRNA is categorized as follows:
++++: indicates a very high level of mRNA
+++: indicates a high level of mRNA
++: indicates are moderate high level of mRNA
+: indicates a relative low level of mRNA
−: no mRNA detectable

Apart from increasing, as outlined herein, the shoot-to-root ratio and seed production by inhibiting the activity of an invertase, more preferably of a root invertase, also plant diseases can be treated and prevented, respectively. The rational underlying this method for the treatment of a plant suffering from a plant disease is that by reducing the carbohydrate supply to the root those pathogens feeding on the root carbohydrates, are deprived of their energy source. Insofar, various diseases caused by various pathogens can be treated whereby the term treatment as preferably used herein also comprises prevention of such disease and thus protection of plants from such disease which are, in principle, susceptible to such disease or are at risk to suffer from such disease. It will be obvious for the one skilled in the art that quite a number of diseases and pathogens can thus be prevented and treated, respectively. Among others, a pathogen is the fungus Plasmodiophora brassicacae which is causing club root disease. This fungus penetrates into the root of brassicacae, whereupon the roots show a significant growth affecting in a negative manner both water and nutrient uptake by the plant. As a consequence, the plantlets grow poorly and the older leaves yellow. The spores of the fungus can survive in soil up to 20 years so that this kind of disease is promoted by an intense crop rotation. Particularly affected plants are cultivated plants such as rapseed, cabbage, radish, mustard and cress, as well as ornamental plants, each preferably of the family brassicacae.

Another pathogen which can thus be prevented and/or treated in accordance with the present invention is the fungus Verticillium which causes, among others, brachiomycosis and affects quite a number of plants, including ornamental plants, ornamental trees, fruit trees, vegetables and field plants. Therefore, the diseases which can be prevented and/or treated in accordance with the present invention are those caused by or associated with Verticillium.

A further group of pathogens which can thus be prevented and/or treated in accordance with the present invention are nematodes which feed from carbohydrates of the roots of host plants. The plants particularly affected by this kind of pathogen are maize, cereals and other monocotolydones and dicotolydones. Therefore, the diseases which can be prevented and/or treated in accordance with the present invention are those caused by or associated with nematodes.

In a preferred embodiment, the invertase which is inhibited by the invertase inhibitor in order to increase shoot-to-root ratio, seed production and resistance of a plant to a disease, more particularly any of the diseases described herein, is an invertase in accordance with the present invention.

The invertases in accordance with the present invention are defined by their nucleic acid sequence as disclosed herein. The invertases in general, have the following amino acid sequence in their catalytic center: cysteine (C)— proline (P)— asparagine (D) which, at the nucleic acid level, corresponds to TGT/C—CCT/C/A/G—GAT/C in case of cell wall invertases, and cysteine (C)— valine (V)—asparagines (D), or at the nucleic acid level, TGT/C, GTT/C/A/G—GAT/C for vacuolar invertases.

The cell wall invertases as disclosed herein can be compared to the six known sequences of invertases from Arabidopsis (AtcwINV 1-6) and it has been found that the cell wall invertases can be linked to the invertases 1 to 4 of Arabidopsis as depicted in table 1.

The vacuolar invertases of the present invention can be aligned to databank entries the result of which is depicted in table 2.

It is also within the present invention that those nucleic acid molecules are comprised which hybridized to the nucleic acid sequences according to SEQ.ID.NO 1 to Z, preferably under stringent conditions. Such stringent conditions are, for example, described in Sambrook J., Fritsch E. F. and Maniatis T. (1989). Molecular cloning. A laboratory manual. Cold Spring Harbor Laboratory, Cold Spring Harbor N.Y.

More preferably RNA samples are fractionated in formaldehyde agarose gels, transferred to nitrocellulose membranes and hybridized to ³²P-labelled cDNA of the corresponding gene under standard conditions (Sambrook et al., 1989), preferably at 42° C.

Furthermore, it is within the present invention that the invention is related to a nucleic acid molecule which, but for the degeneracy of the genetic code, would hybridize, preferably under stringent conditions, to the nucleic acid molecules disclosed herein, each preferably coding for invertase.

It is also within the present invention that the respective nucleic acid molecules either coding for an inhibitor of an invertase or coding for the invertase according to the present invention, are cloned into a vector. Preferably such vector is an expression vector. For the purpose of producing any polypeptide encoded by the nucleic acids according to the present invention or those described herein an expression vector is used, whereby such expression vector is a viral, microbial, plant or animal vector, preferably a plant vector. It is also within the present invention that such vector is inserted into a cell, whereby such cell is preferably a plant cell. It is also within the present invention that the plant cell is grown into a mature plant. In a further embodiment the cell and the mature plant generate a seed containing such vector or a cell containing such vector. Preferably the seed and/or the plant is a hybrid plant which is preferably not capable of being propagated by common biological means, i.e. crossing and propagation.

It is also within the present invention that the vector contains a root-specific promoter as preferably described herein.

In a further aspect, the invention is related to a nucleic acid construct which comprises a root-specific promoter and a nucleic acid coding for an invertase inhibitor. The root-specific promoter may be any of the promoters described herein. The invertase inhibitor is preferably any invertase inhibitor described herein. Similar to the vector, the root-specific promoter and the nucleic acid coding for the invertase inhibitor are operably linked to each other allowing for the expression of the invertase inhibitor. This nucleic acid construct can be introduced to a vector and cell, respectively, as defined above. Preferably such cell is regenerated to a tissue and plant, respectively and a plant regenerated or obtained therefrom by both genetic engineering means as well as conventional propagation. Such plant may be any plant and any species and family or genus, as described herein.

Methods for the introduction of this genetic construct and vector, respectively, into a plant cell, which is preferably an embryonic plant cell, are known to the one skilled in the art and, for example, described in Clough S. J. and Bent A. F. (1998). Floral dip: a simplified method for Agrobacterium-mediated transformation of Arabidopsis thaliana. Plant J. 16, 735-743. Preferred transformations methods are Agro, particle bombardment, and floral dip. More preferably, Arabidopsis plants are transformed by floral dip according to the method of Clough and Bent (1998). Plants are grown under long days until flowering and clipped to encourage proliferation of secondary bolts. An Agrobacterium strain, carrying the construct in a binary vector, is grown in a large culture in YEB at 28° C. Subsequently, the Agrobacterium is centrifuged and resuspended to a OD_{600 nm}=0.8 in 5% sucrose solution. Silwett is added to a concentration of 0.05% and flowers are dipped by immersion in the solution for 2-3 seconds. Dipped plants are covered with Saran wrap during 24 hours, and in darkness, to maintain high humidity. Subsequently, they are uncovered and grow normally. Watering is stopped as seeds become mature and seeds are selected in antibiotic-containing plates.

In another aspect, the invention is also related to this kind of plant which is preferably a transgenic plant. In a particularly preferred embodiment, the genetic construct is under control of an inducible promoter as known to the one skilled in the art. Particularly preferred inducible promoters are, for example, but not limited to, a dexamethasone inducible promoter such as described in Aoyama T., et al. (Aoyama T. and Chua N. H. (1997). A glucocorticoid-mediated transcriptional induction system in transgenic plants. Plant J. 11, 605-612), or McNellis T. W. et al. (McNellis T. W., Mudgett M. B., Li K., Aoyama T., Horvath D., Chua N.-H. and Staskawicz B. J. (1998). Glucocorticoid-inducible expression of a bacterial avirulence gene in transgenic Arabidopsis induced hypersensitive cell death. Plant J. 14, 247-257), or a steroid-inducible promoter such as described in Schena M. et al. (Schena M., Lloyd A. M. and Davis R. W. (1991). A steroid-inducible gene expression system for plant cells. Proc. Natl. Acad. Sci. USA 88, 10421-10425), or Zuo J. et al. (Zuo J., Niu Q. W. and Chua N. H. (2000). An estrogen receptor-based transactivator XVE mediates highly inducible gene expression in transgenic plants. Plant J. 24, 265-273).

In a further aspect, the present invention is related to seeds derived from such plant, preferably recombinant plant.

In a further aspect of the present invention, the nucleic acids, vectors, cells and plants as well as other organisms containing such vectors encoding for any of the invertases as disclosed herein, are used for the production of the respective invertase. For such purpose, the respective cells expressing the nucleic acid coding for the invertase is cultivated in an appropriate reaction vessel containing an appropriate medium and subsequently the invertase is isolated and/or purified. Such cultivation and isolation/purification methods are known to the one skilled in the art.

In a still further aspect the present invention is related to the use of any of the invertases as disclosed herein, as a target molecule. A target molecule as used herein is a molecule which is either targeted in vivo or in vitro or in silico. Such targeting can, in a preferred embodiment, mean that the invertase is subject to a screening process, preferably an in vitro screening process or an in silico screening process.

In connection with the in vitro screening process, the target molecule as such is provided and one or several compounds, preferably taken from a library, are tested typically by contacting the compound with the target molecule, whether or not any of the compounds have an impact on the target, preferably whether or not there is an increase or decrease in the activity of the target, i.e. the invertase activity. As the target molecule is an invertase, assays are known to the one skilled in the art to evaluate whether a compound, also referred to herein as a candidate compound, has an inhibitory or activating effect on the invertase. Such molecules can further be used as a candidate, lead or compound for the manufacture of an agrochemical product. Preferably such agrochemical product is an agrochemical suitable to increased shoot-to-root ratio, seed production and/or increasing resistance if, preferably if it shows an inhibitory effect on the invertase.

In an in silico screening, the target molecule, i.e. any of the invertases as described herein, is used as a molecule against which the fit of other molecules is tested or other molecules are designed by means of computational analysis and design so as to fit to the target molecule preferably such as to inhibit or promote the activity of the target molecule. Preferably such fitting is related to the active center of the invertase. The thus identified compound, either identified by in vitro and/or by in silico screening can ultimately be used in connection with the methods disclosed herein. The thus identified compound can thus be a plant growth promoter or a plant protective agent, particularly in case such compound is actually decreasing the activity of an invertase, more preferably a root invertase and its activity respectively, in accordance with the present invention.

In a preferred embodiment of any aspect of the present invention the invertase is a plant invertase, and an inhibitor to an invertase is an inhibitor to plant invertase, whereby the inhibitor is a plant or plant-derived inhibitor.

The invention is now further illustrated by the attached figures, examples and the sequence listing from which further features, embodiments and advantages may be taken.

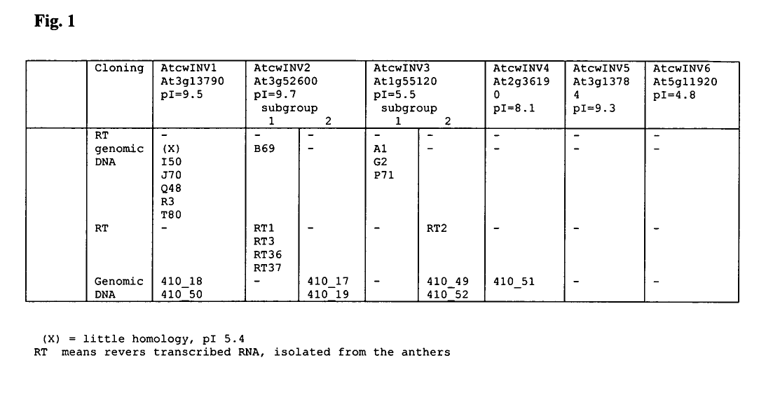

FIG. 1 shows table 1 attributing the invertases of the present invention to known invertases from Arabidopsis;

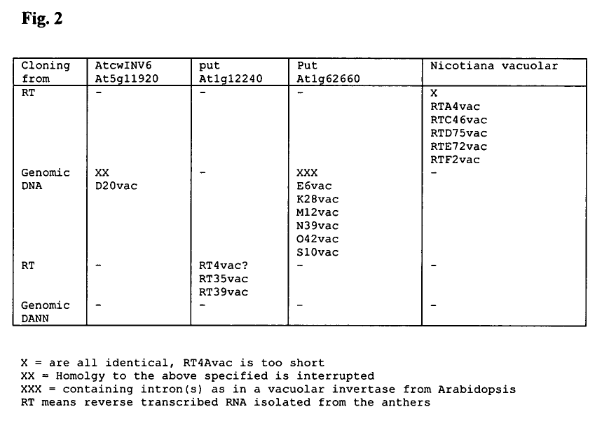

FIG. 2 shows table 2 attributing the invertases of the present invention to known invertases indicating those entries of databank having the highest homology with the invertases according to the present invention;

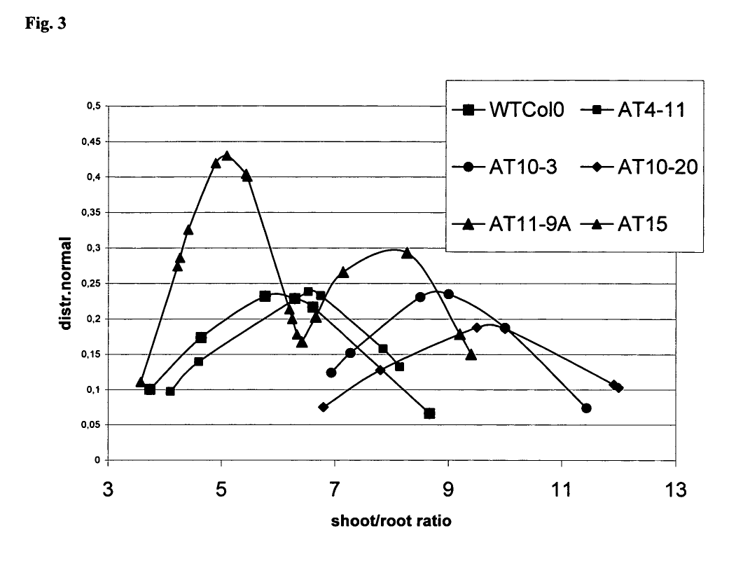

FIG. 3 is a diagram indicating the distribution of plants as a function of shoot-to-root ratio for the various transgenic plants (AT) and the corresponding wild type plants (WTCol10) cultivated in medium containing 1% sucrose, whereby the overall number of plants of a distinct variety is set to 1 (100%) and the relative portion of plants is indicated which have a specific shoot-to-root ratio;

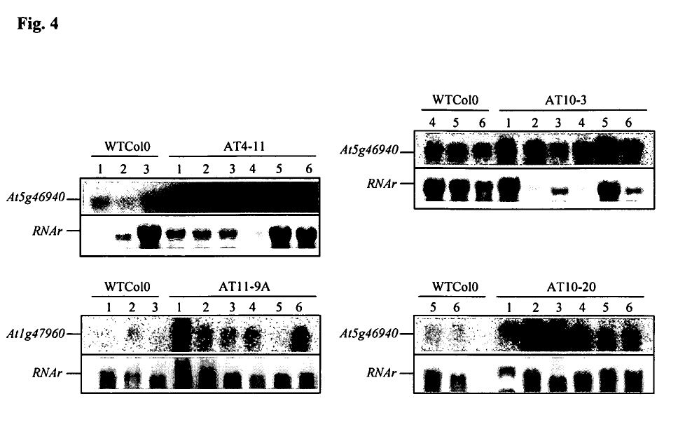

FIG. 4 is the result of a Northern blot analysis representing RNA expression of invertase inhibitors in roots of transgenic plants in comparison to control wild type plants;

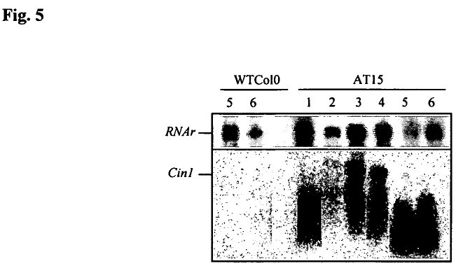

FIG. 5 is the result of a Northern blot analysis of RNA expression of Cin1 in roots of transgenic plants;

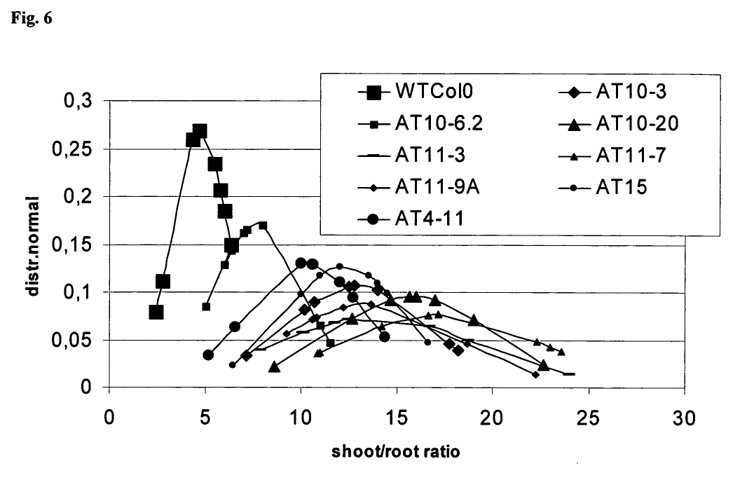

FIG. 6 is a diagram, similar to the one of FIG. 3, indicating the distribution of plants as a function of shoot-to-root ratio for the various transgenic plants (AT) and the corresponding wild type plants (WTCol10) cultivated in perlite;

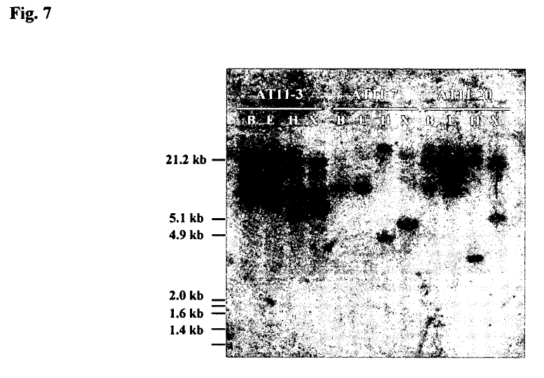

FIG. 7 is the result of a Southern blot analysis of different independent lines containing different copy number and insertion sites for AtC/Vif1 (At1g47960);

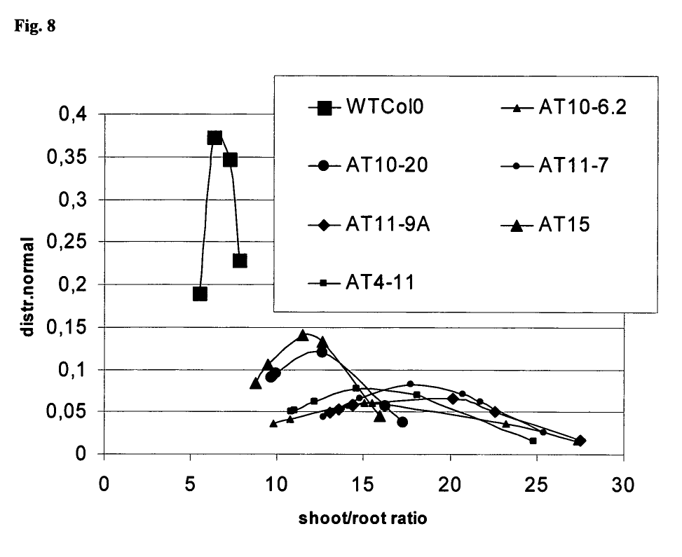

FIG. 8 is a diagram, similar to the one of FIG. 3, indicating the distribution of plants as a function of shoot-to-root ratio for various transgenic plants (AT) and the corresponding wild type plants (WTCol10) initially grown in medium containing 1% sucrose and subsequently transferred to perlite;

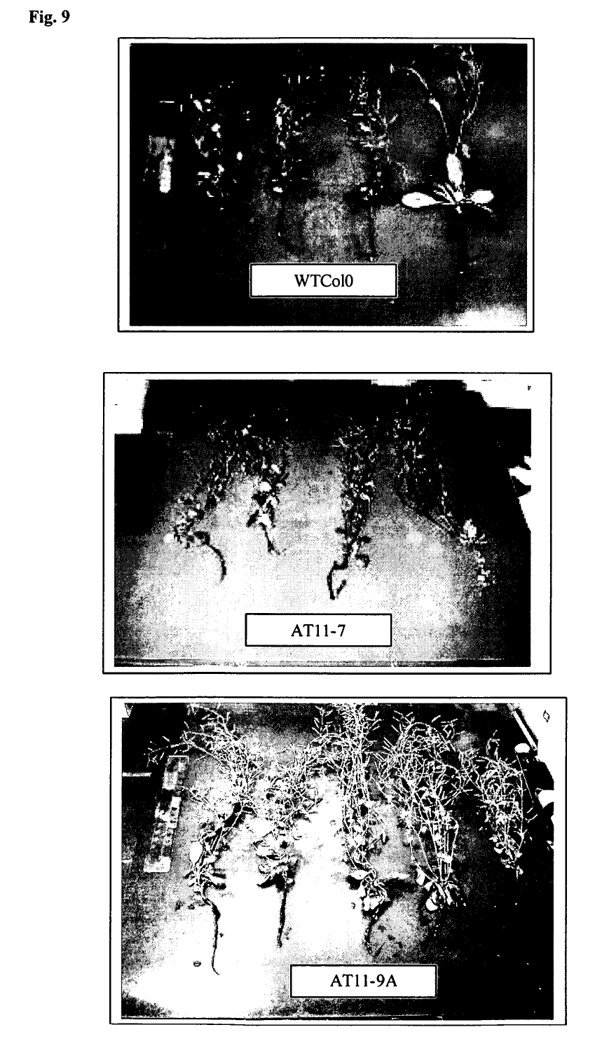

FIG. 9 is a photograph of the phenotype of wild type and transgenic plants after 49 days of growth at LD conditions;

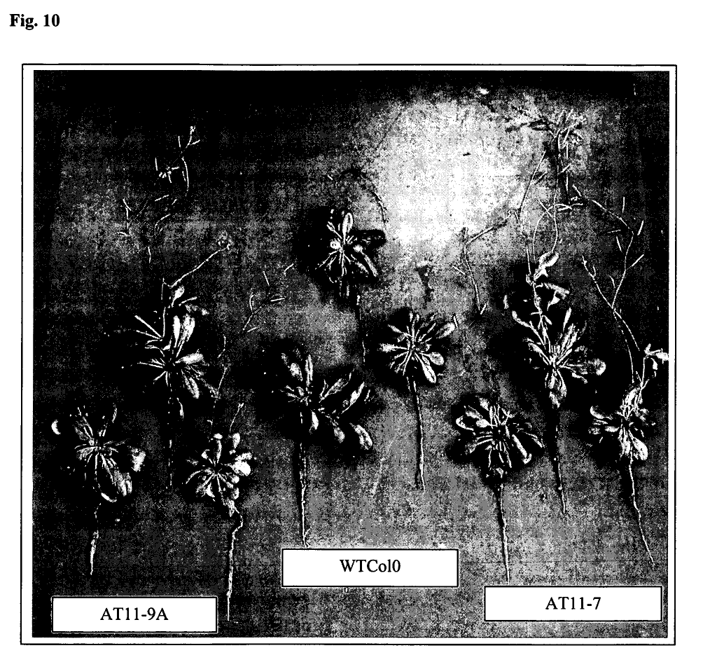

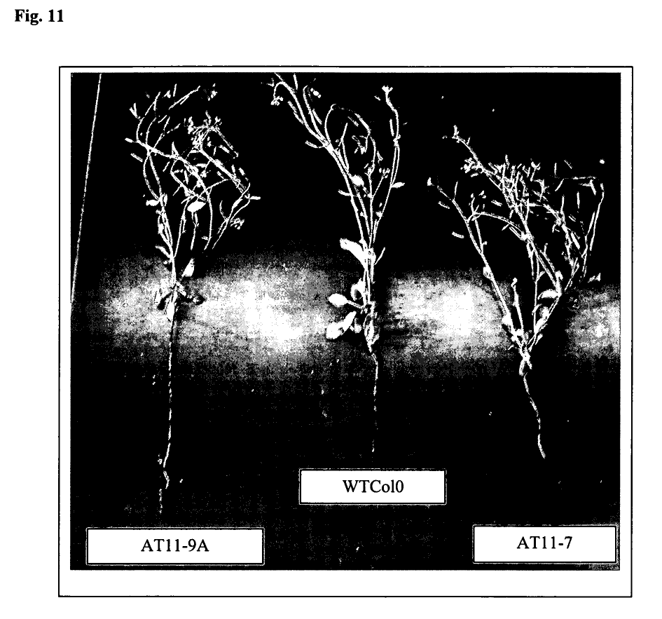

FIG. 10 is a photograph of the phenotype of wild type and transgenic plants after pre-growing in selection medium containing glucose and transfer to perlite after 14 days taken after 43 days of growth at SD conditions;

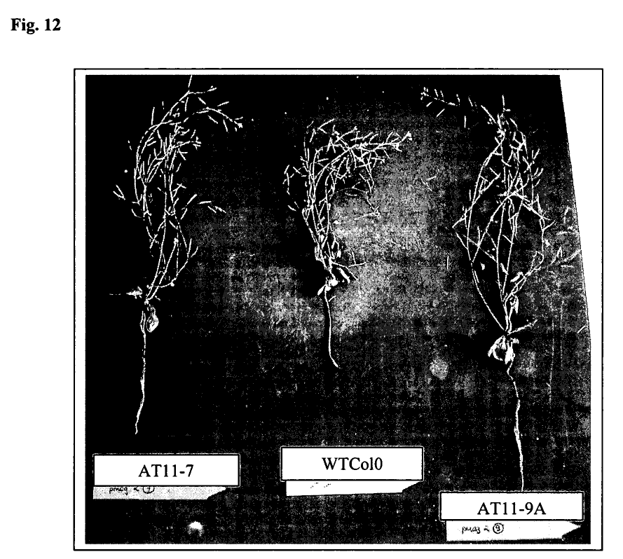

FIG. 11 is a photograph of the phenotype of wild type and transgenic plants after pre-growing in selection medium containing glucose and transfer to perlite after 14 days, whereby the photographs were taken after 30 days of growth at LD conditions;

FIG. 12 is a photograph of the phenotype of wild type and transgenic plants after pre-growing in selection medium containing glucose and transfer to perlite after 14 days, whereby the photographs were obtained after 40 days of growth at LD conditions;

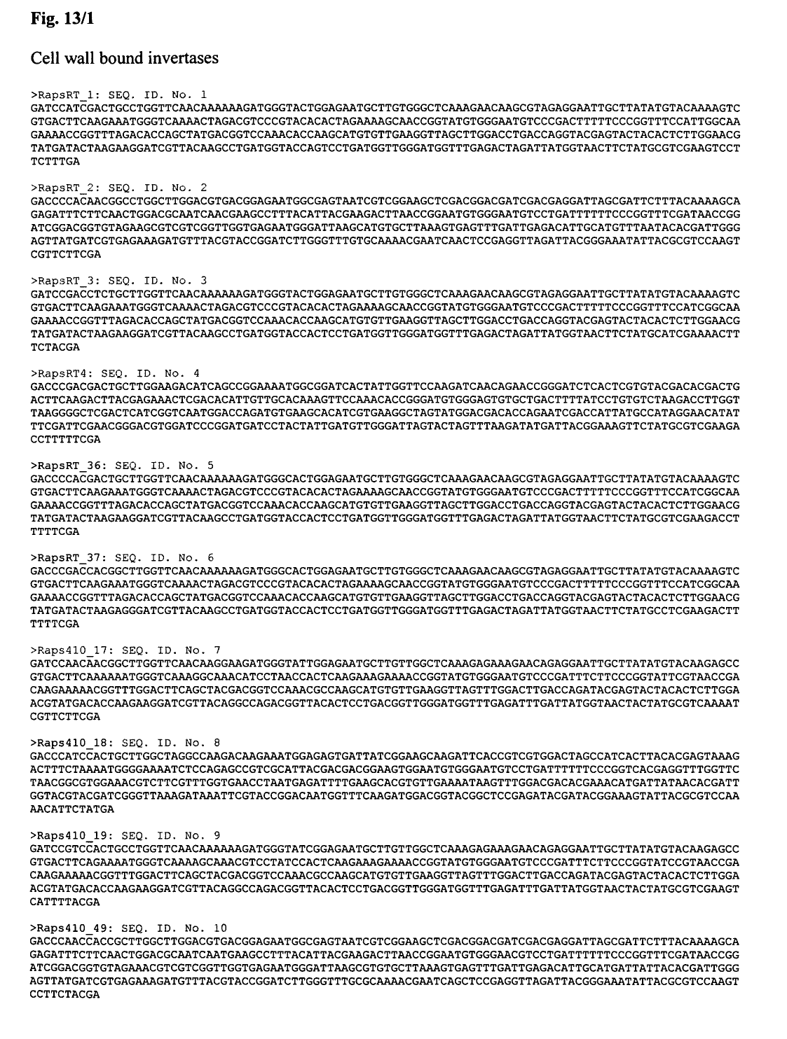

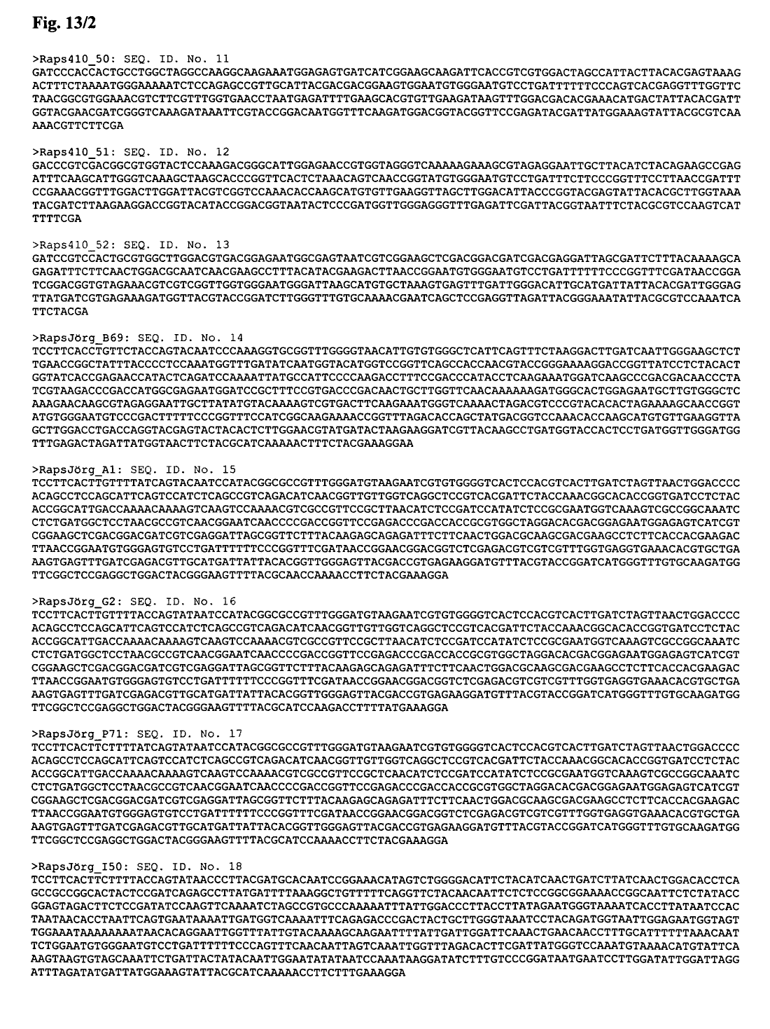

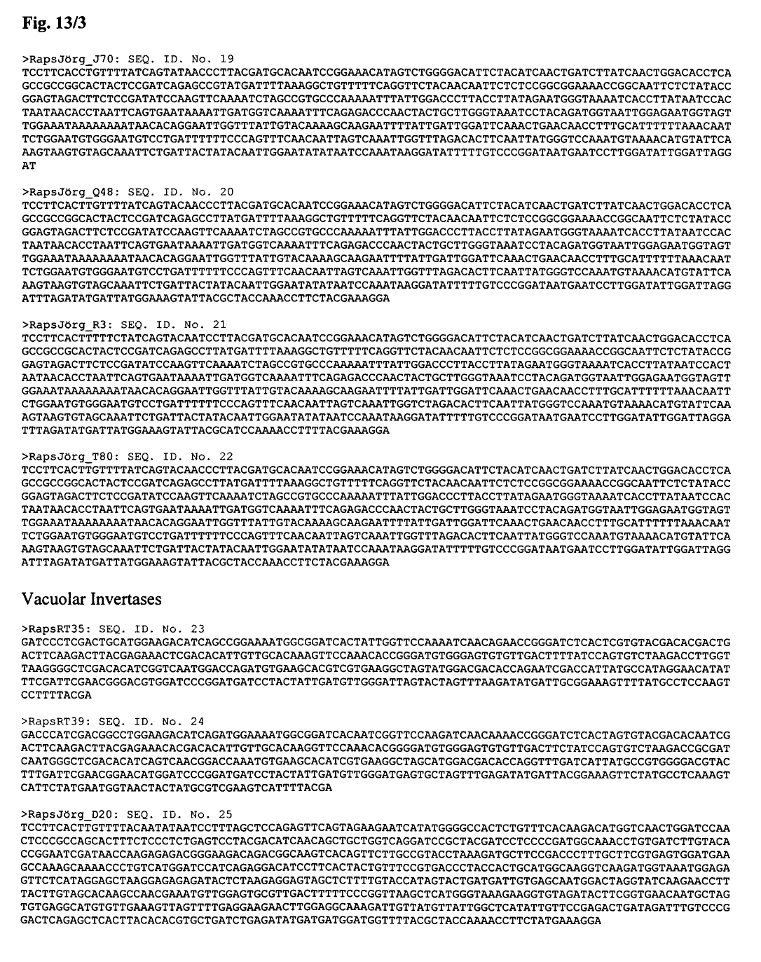

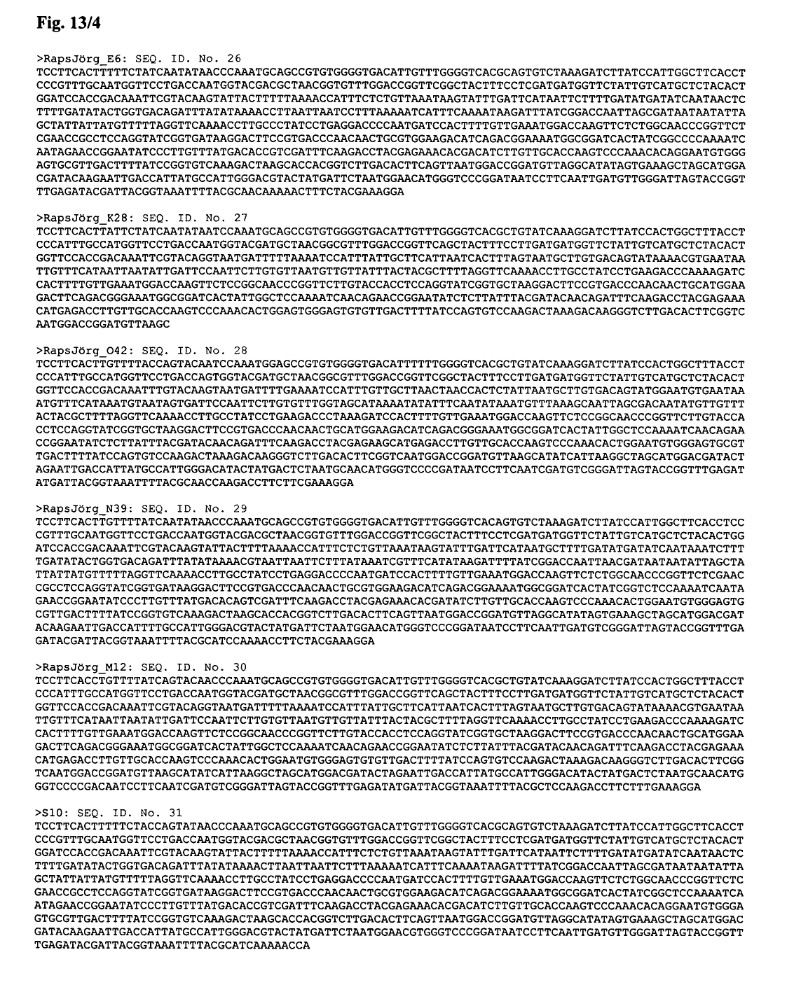

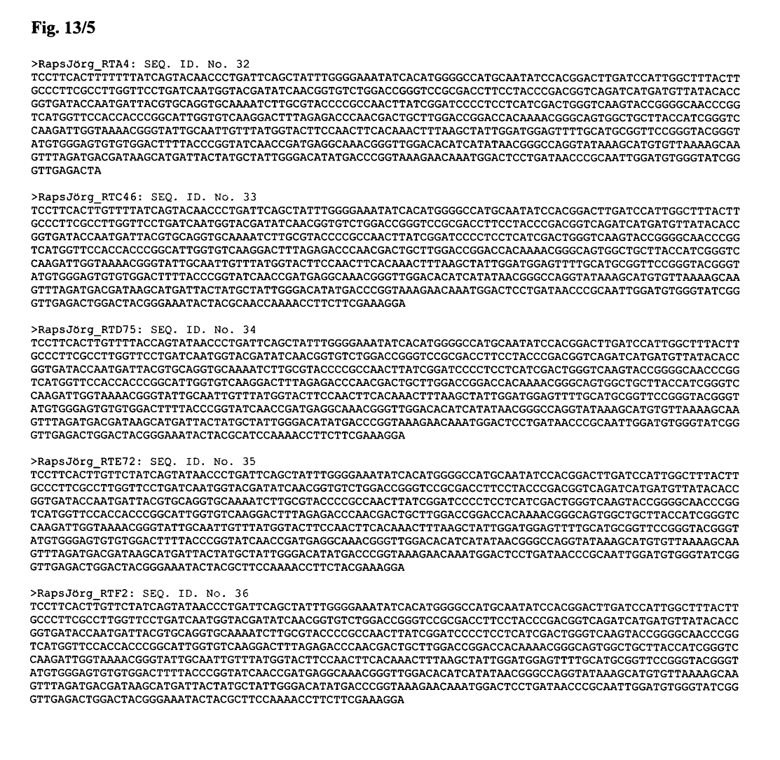

FIG. 13 indicates the internal references and the nucleic acid sequences of the invertases in accordance with the present invention;

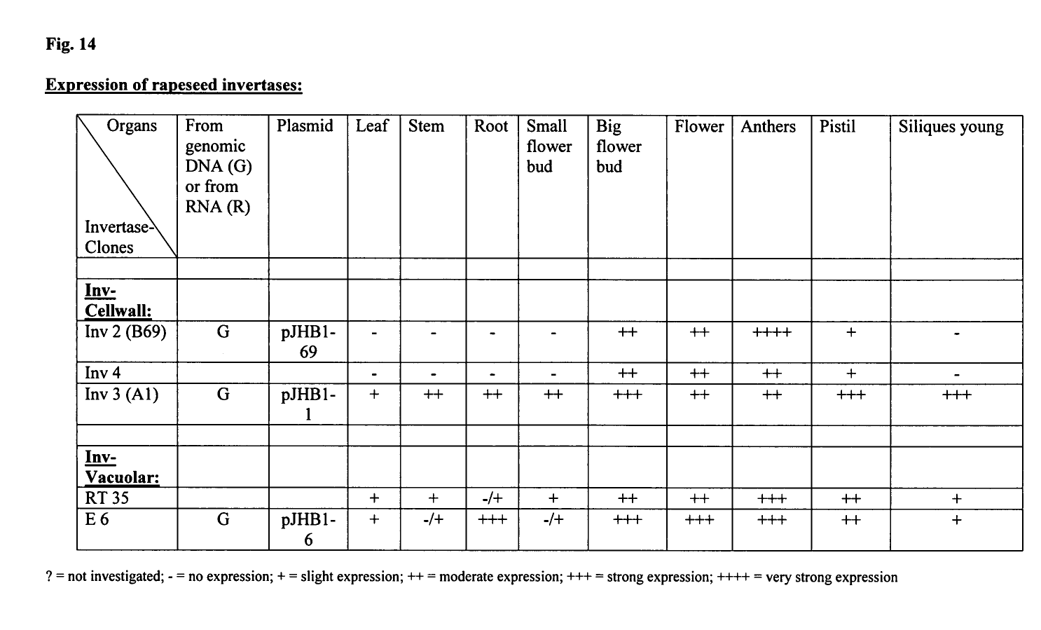

FIG. 14 shows a table indicating the expression level of different rapeseed invertases in different plant organs indicating that cell wall invertase Inv 3(A1) and invertase E6 are particularly preferred invertases for the practice of the present invention;

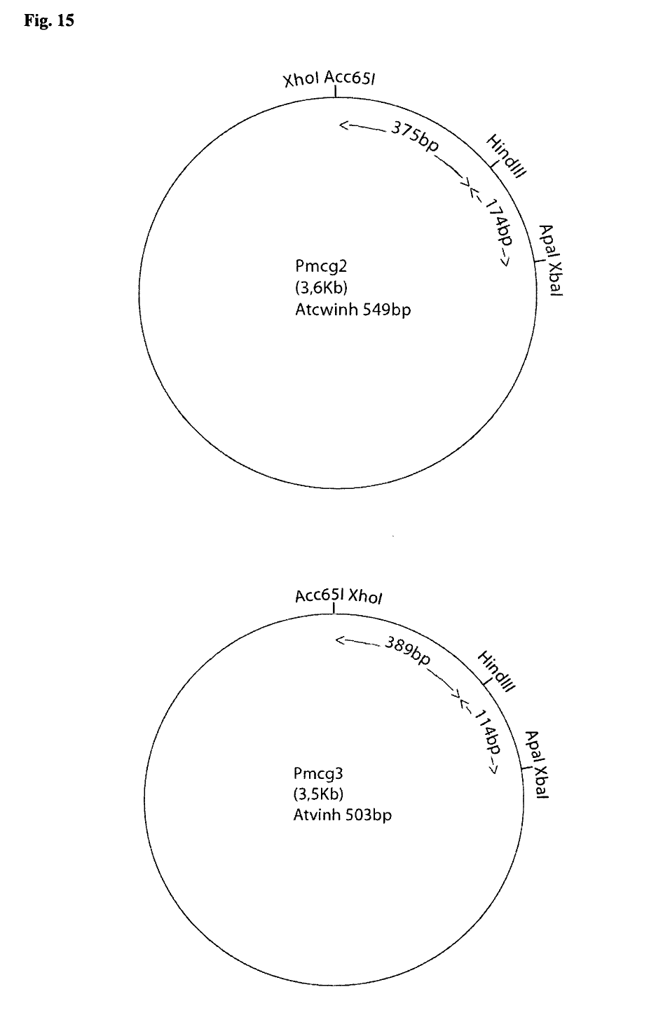

FIG. 15 shows a restriction map for plasmids pmcg2 and pmcg3;

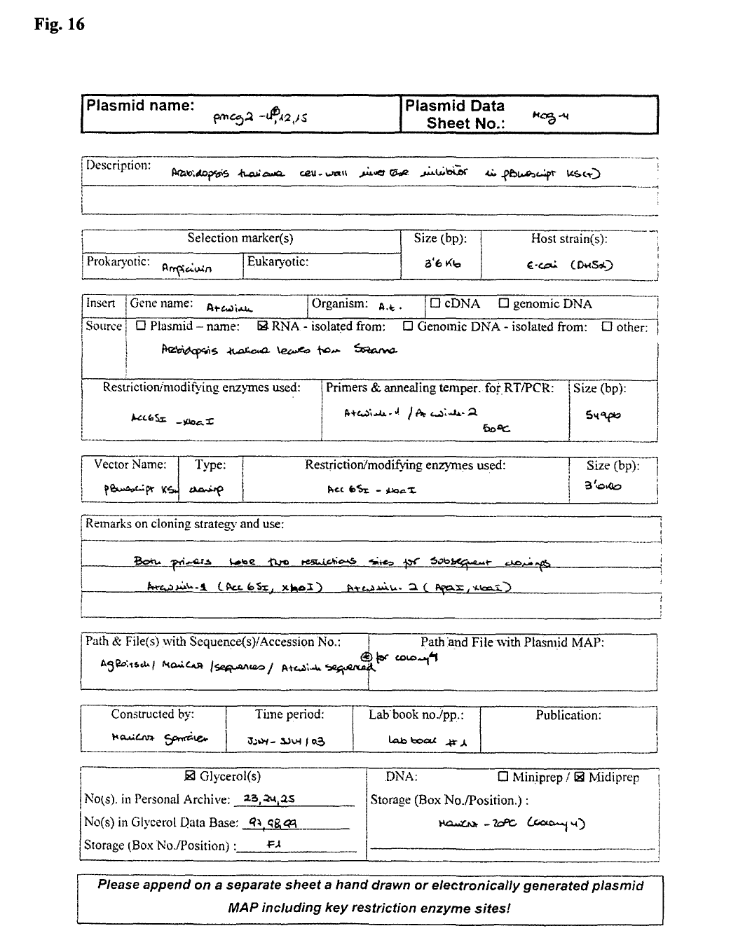

FIG. 16 is the plasmid data sheet for MCG-4;

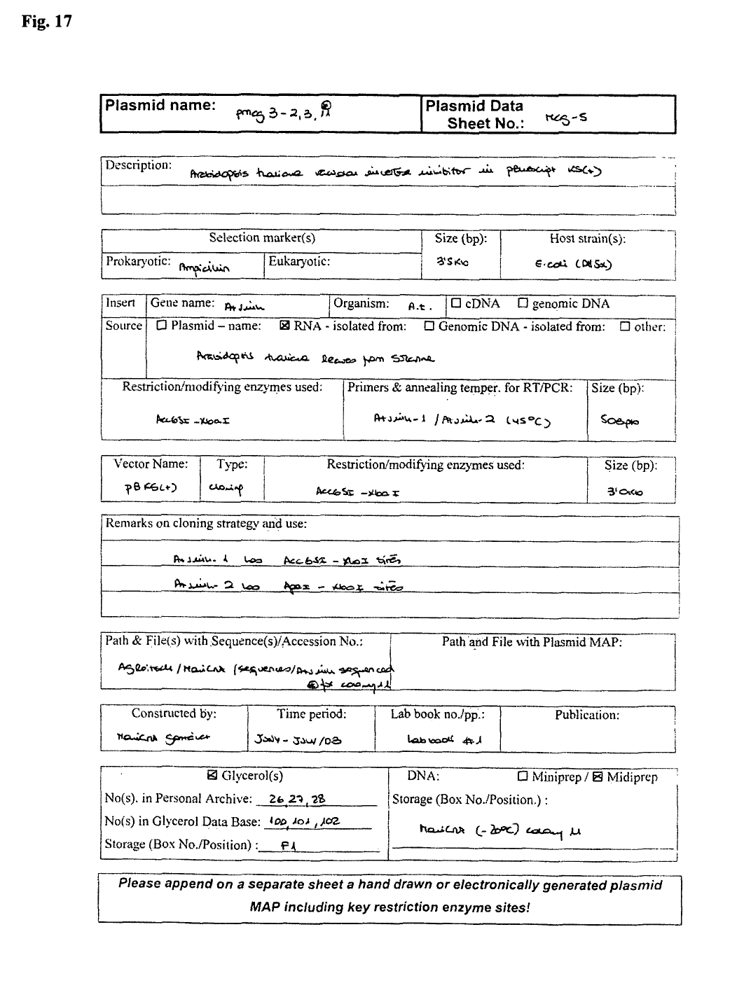

FIG. 17 is the plasmid data sheet for MCG-5;

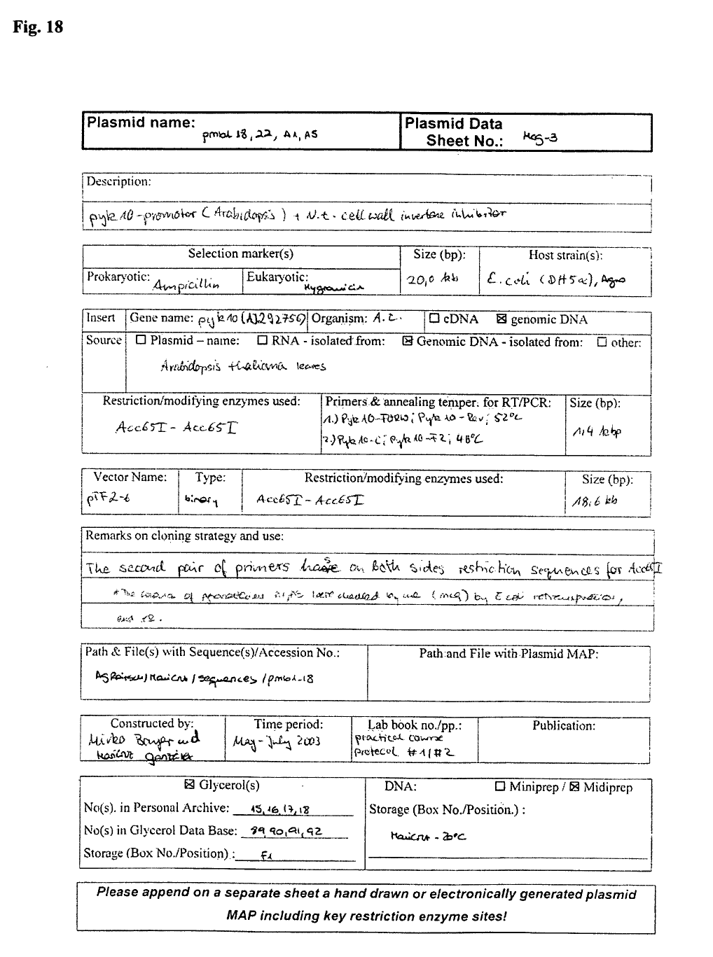

FIG. 18 is the plasmid data sheet for MCG-3;

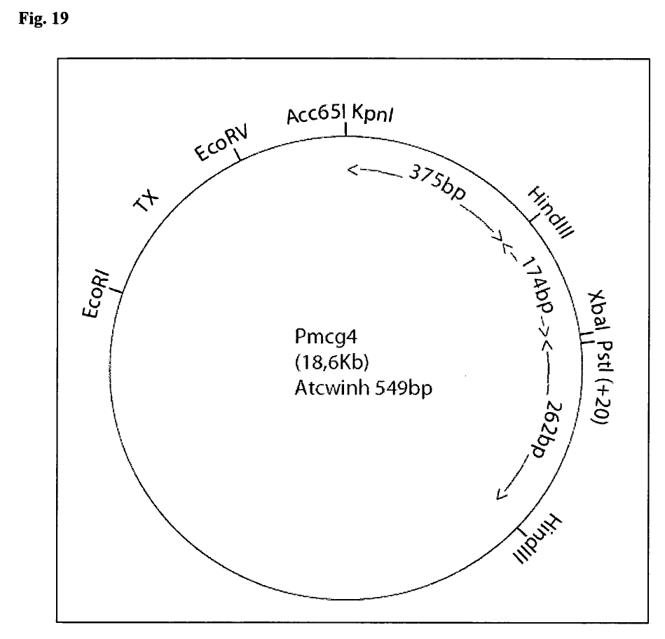

FIG. 19 is a schematic illustrating the generation of pmcg4;

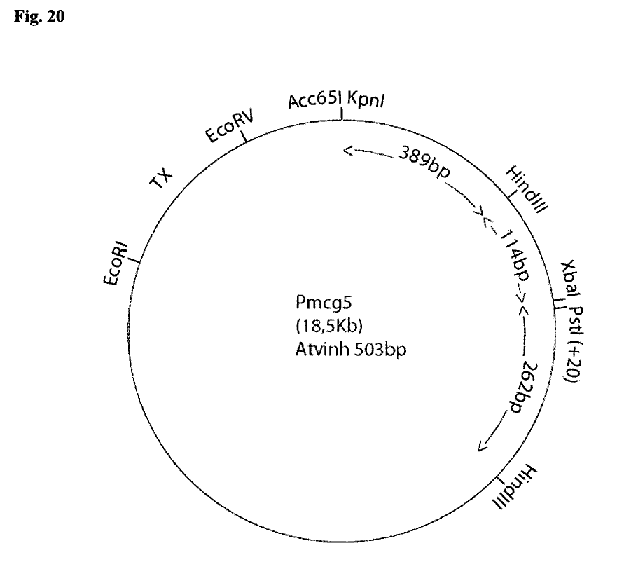

FIG. 20 is a schematic illustrating the generation of pmcg5;

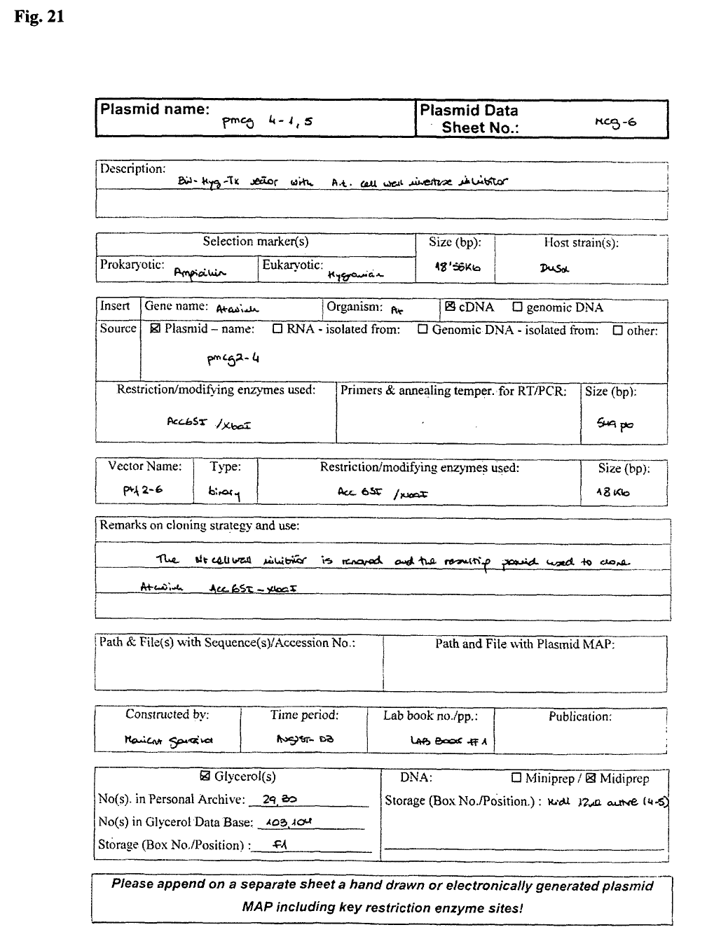

FIG. 21 is the plasmid data sheet for MCG-6;

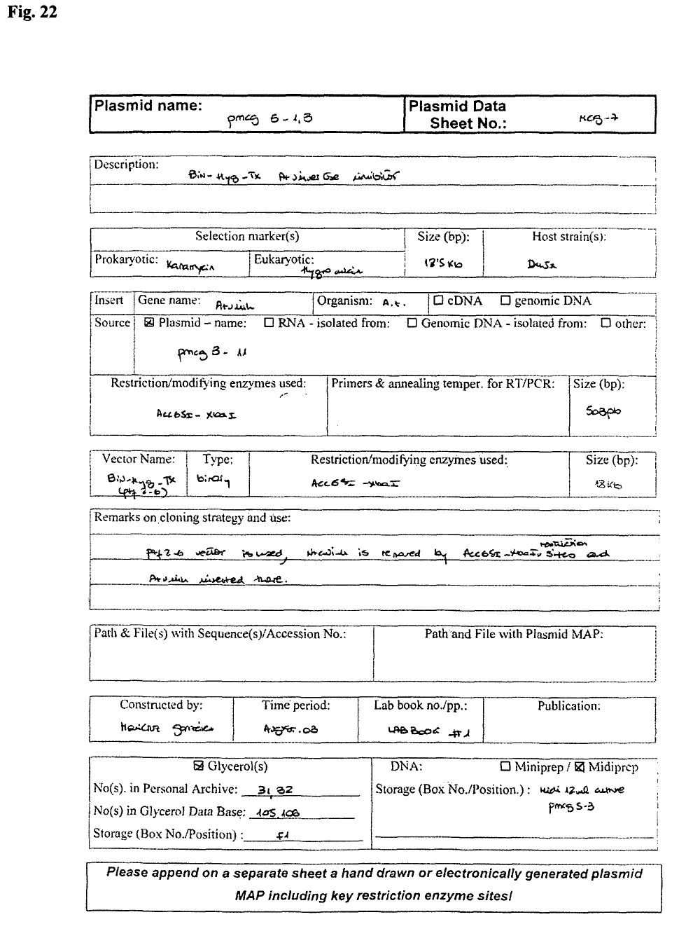

FIG. 22 is the plasmid data sheet for MCG-7;

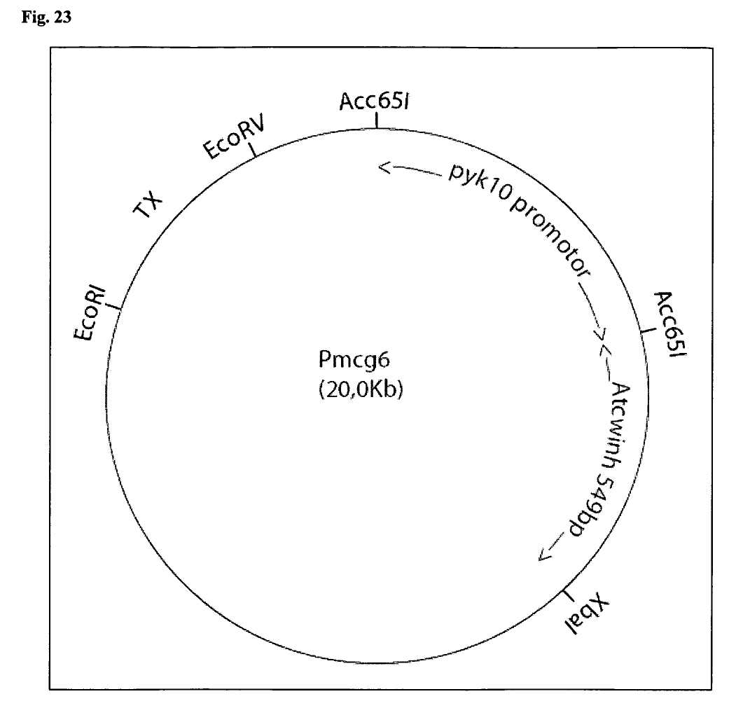

FIG. 23 is a schematic illustrating the generation of pmcg6;

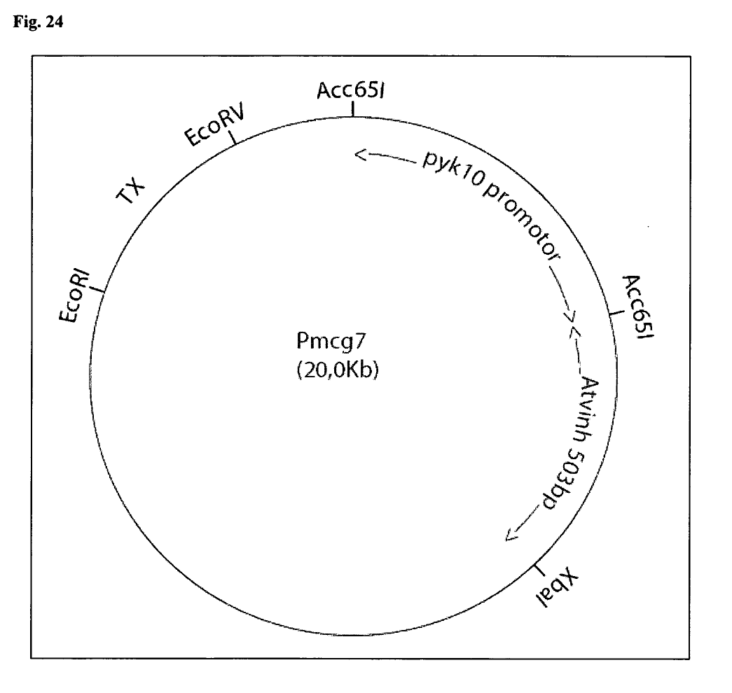

FIG. 24 is a schematic illustrating the generation of pmcg7;

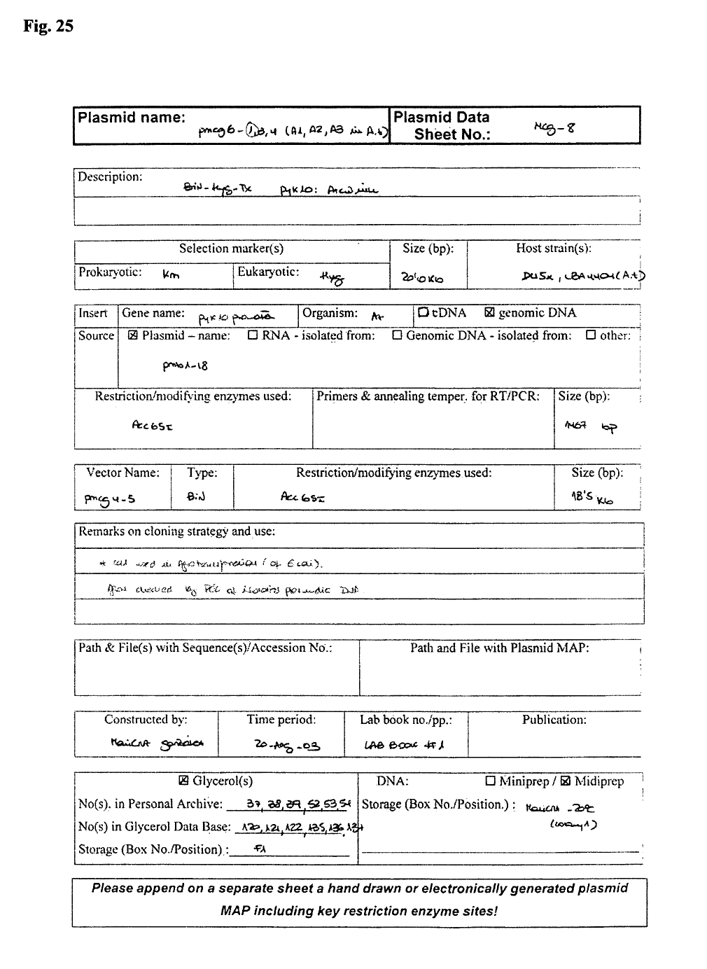

FIG. 25 is the plasmid data sheet for MCG-8;

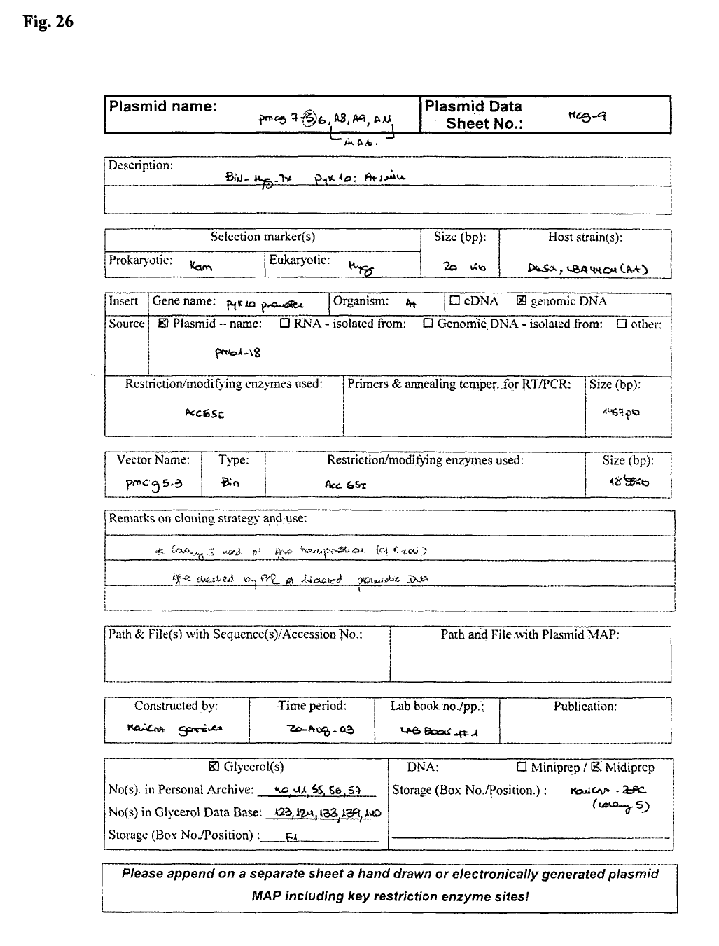

FIG. 26 is the plasmid data sheet for MCG-9;

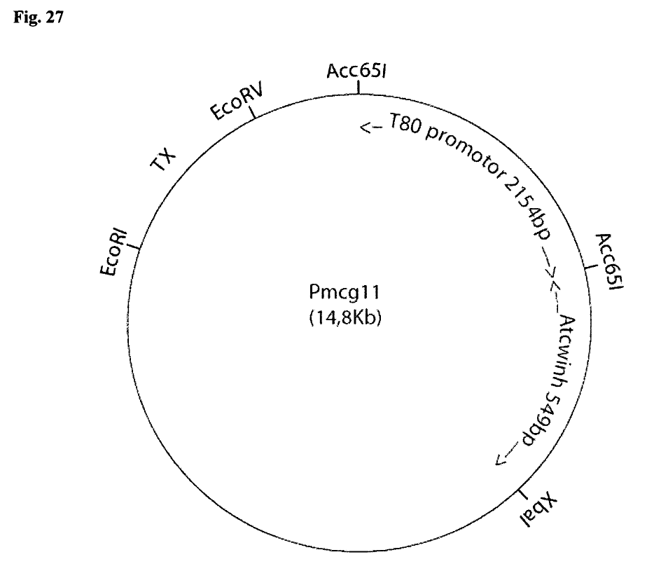

FIG. 27 is a schematic illustrating the generation of pcmg 11-9;

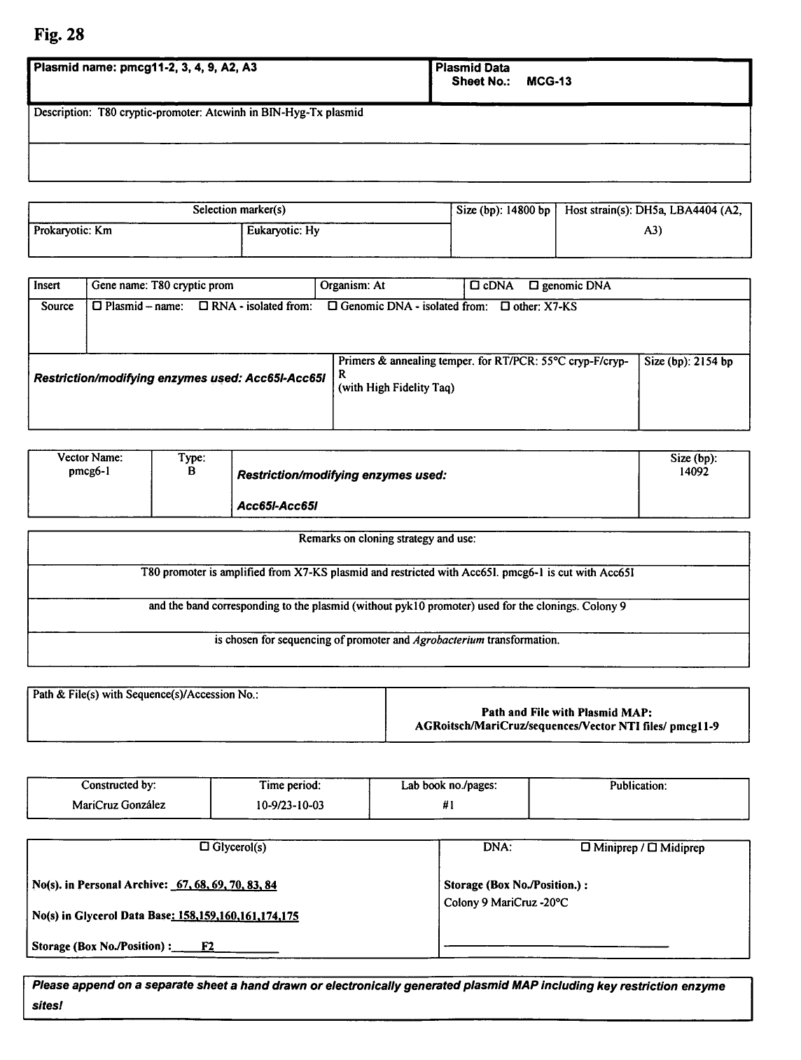

FIG. 28 is the plasmid data sheet for MCG-13

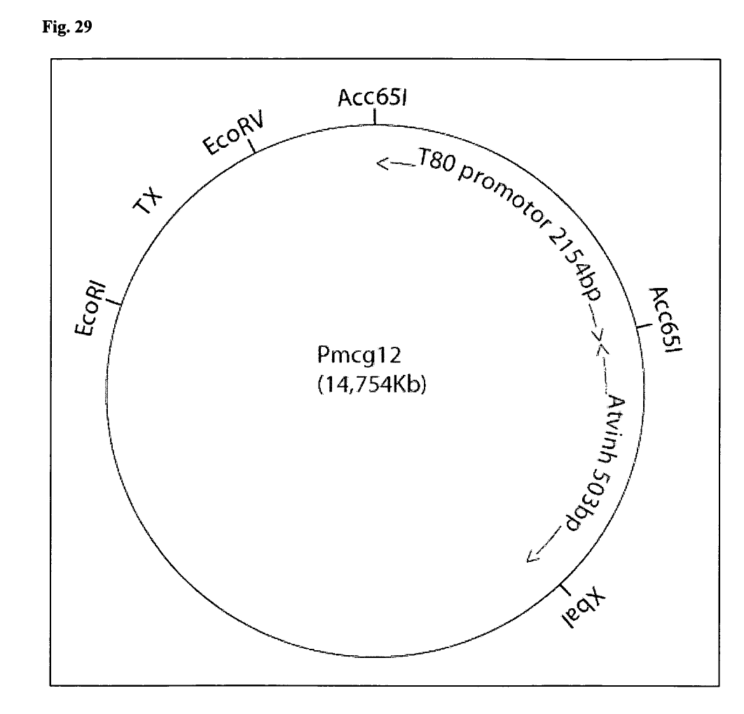

FIG. 29 is a schematic illustrating the generation of pmcg 12-1

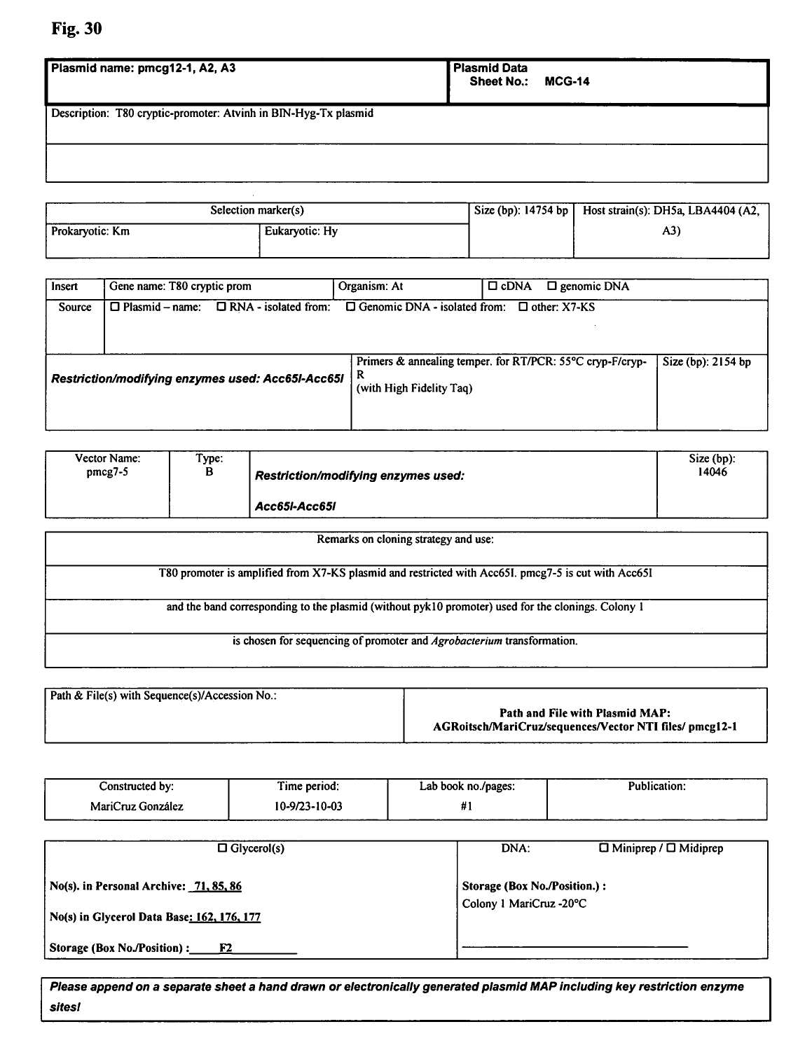

FIG. 30 is the plasmid data sheet for MCG-19;

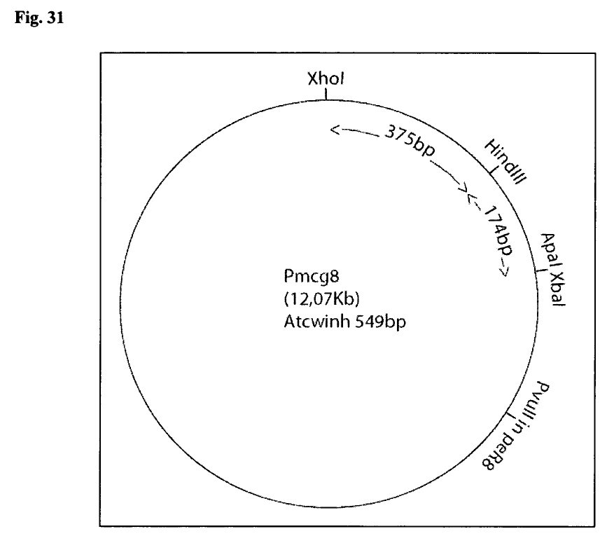

FIG. 31 is a schematic illustrating the generation of pcmg 8;

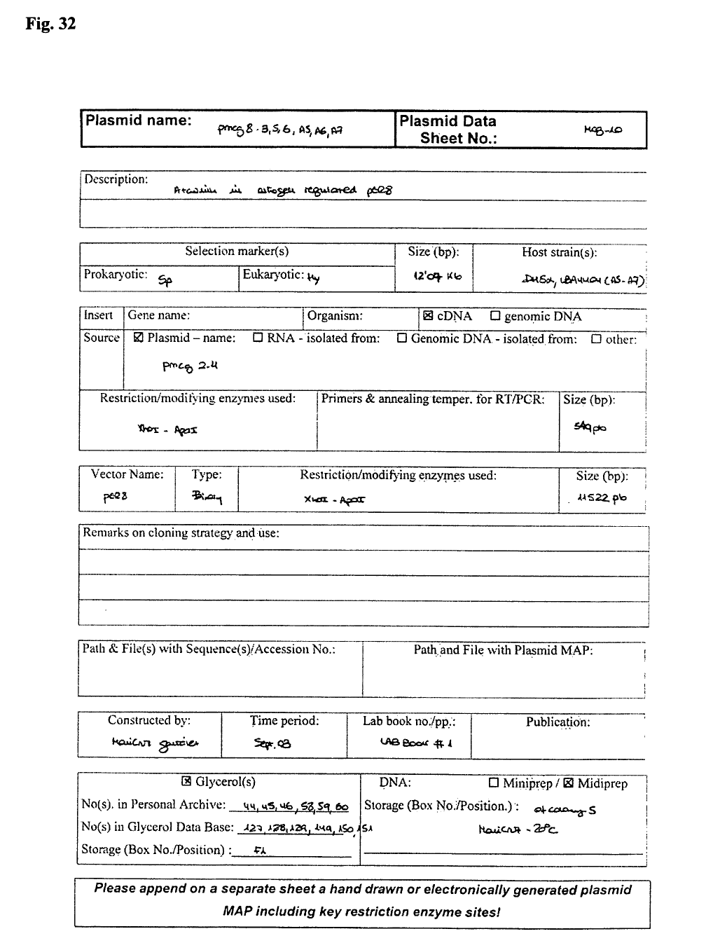

FIG. 32 is the plasmid data sheet for MCG-10;

FIG. 33 is the plasmid data sheet for MCG-11;

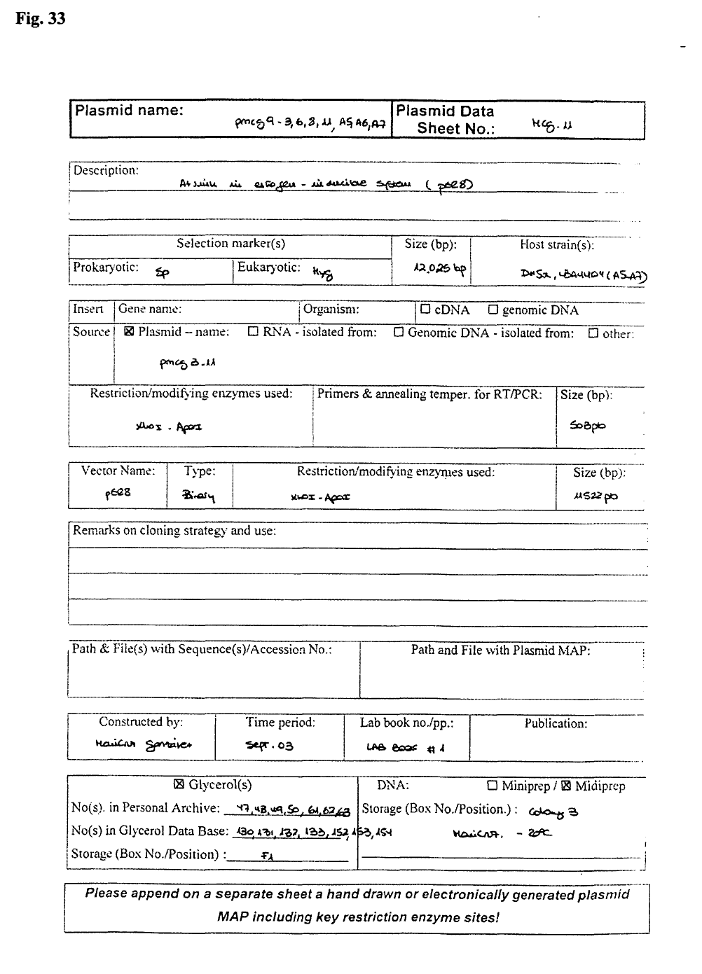

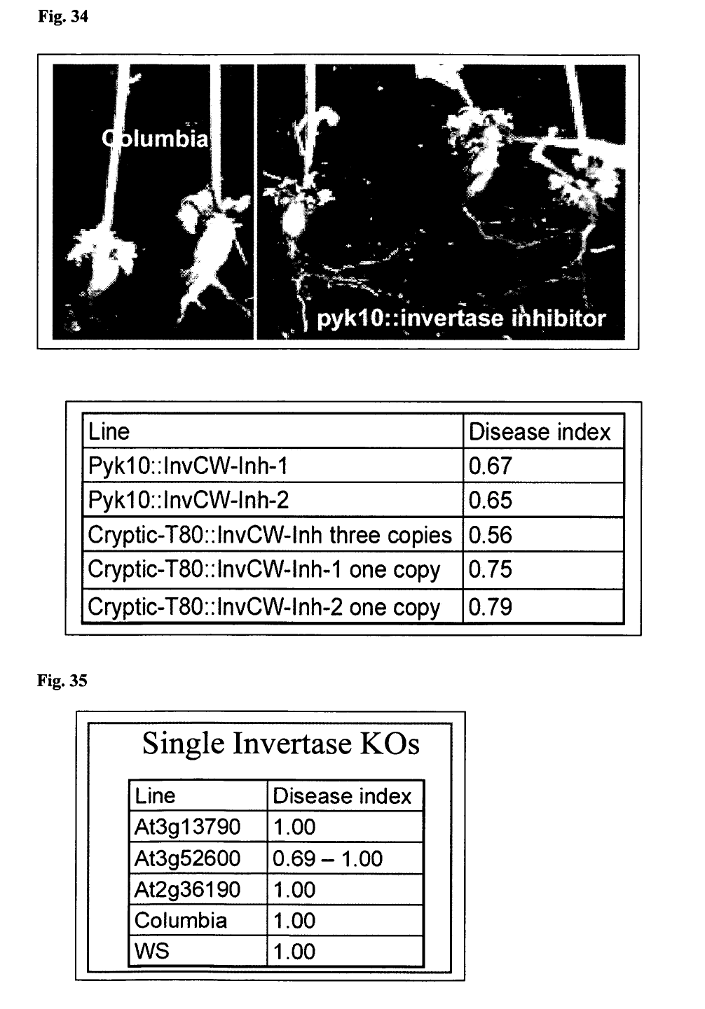

FIG. 34 is a photograph showing representative examples of an infection of wild type plants (Columbia) and transgenic plants expressing the Arabidopsis invertase inhibitor AtCNVIF2, At5g46940 under control of the pyk10 promoter (pyk10:invertase inhibitor) by Plasmodiophora brassicae; whereas the roots of the wildtype plants show severe disease symptoms such as extensive swelling (left), the roots of the transgenic plants are essentially symptom free except for the hypocotyl region, a region where the promoter is not expressed. (A);
and a table indicating the disease index according to Siemens et al. (Siemens et al., 2002) a quantification of the degree of symptoms, of various recombinant A. thaliana strains where infected by Plasmodiophora brassicae; in comparison with wildtype control plants (disease index 1) the plants of individual transgenic lines expressing the Arabidopsis invertase inhibitor AtCNVIF2 either under control of the pyk10 promoter or the cryptic-T80 promoter are strongly affected; the data indicate a positive correlation between copy number and protection (B);

FIG. 35 represents a table where the effect of single invertase knock-outs (KO) on the disease index is indicated for several cell lines;

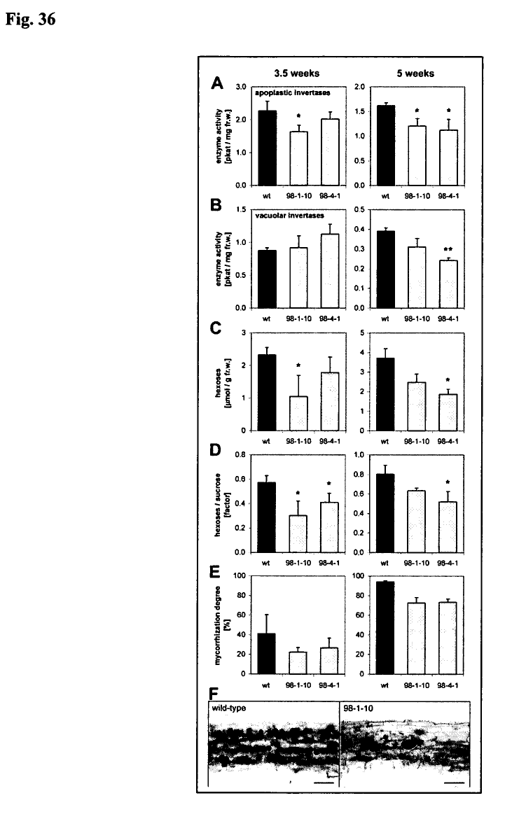

FIG. 36 shows diagrams indicating the activity of various invertases (FIGS. 36 A; B;), the glucose and fructose content of the roots (FIG. 36 C), the ratio of the glucose and fructose contents to the sucrose content of the roots (FIG. 36 D), the degree of mycorrhization (FIG. 36 E), each after 3.5 and 5 weeks, respectively, of infection with G. intraradices in wildtype and two plant lines of A. thaliana, whereby Inylnh stands for invertase inhibitor, and a microphotograph of ink-stained fungal structure in a wildtype and one of the cell lines subject to FIG. 36 A to E (FIG. 36 F);

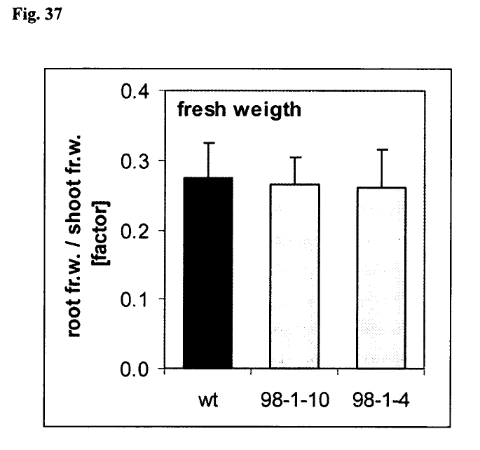

FIG. 37 shows a diagram indicating the result of a biomass analysis for two different recombinant tobacco lines expressing the invertase inhibitor AtCNVIF2 (98-1-10, 98-1-4) and wildtype tobacco plants; and whereby the Fig. shows the root-to-shoot ratio; and

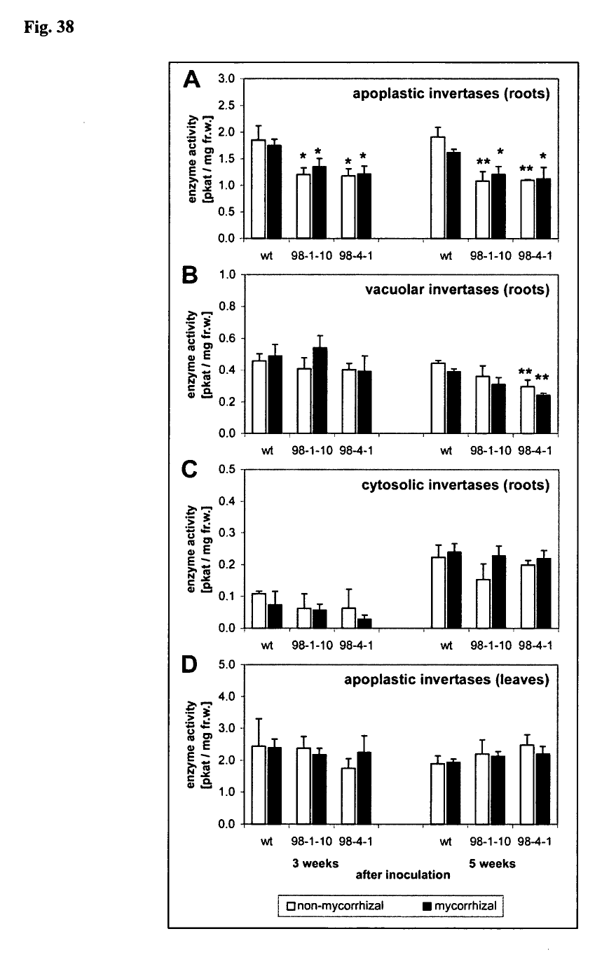

FIG. 38 shows various diagrams indicating the results of the determination of the activities of different invertase isoenzymes such as apoplastic invertase, vacuolar invertase, and cytosolic invertase, in roots and leaves of wildtype tobacco plants (wt) and two different recombinant tobacco lines expressing the invertase inhibitor AtC/VIF2 (98-1-10, 98-1-4).

### EXAMPLE 1

### Generation of Transgenic Plant Cells

In this approach, the cell wall invertase from Chenopodium rubrum was used in order to prevent possible inhibition of Arabidopsis thaliana genes by the plant invertase inhibitors. Different invertase inhibitors were used for the reduction of plant invertase activity in the root: a tobacco cell wall invertase inhibitor (Greiner et al., 1998), and two genes from Arabidopsis with higher homology to cell-wall (At5g46940) and vacuolar invertase inhibitor (At1g47960). Since similar results were obtained for the tobacco and Arabidopsis invertase inhibitors, we focused on the two Arabidopsis genes for all the subsequent approaches. Recently, evidence of in vitro proof of function has been obtained for these two invertase inhibitors, with AtC/VIF1 (At1g47960) specifically inhibiting vacuolar invertase activity, whereas AtC/VIF2 (AT5g46940) inhibits both although with a ten fold higher affinity for vacuolar than for cell wall invertase (Link et al., 2004). But so far, no proof of in vivo activity has been shown. The mentioned genes were engineered under the control of two different Arabidopsis root specific promoters described in the literature: the pyk10 promoter of an Arabidopsis mirosinase (Nitz et al., 2001), and a cryptic promoter reported by Mollier et al. (2000) which is also referred to herein as “crypticT80” or promoter T80-cryptic. In addition, an inducible system in which the expression of the transgene is induced by steroids has also been used for the generation of transgenic plants (Zuo et al., 2000). Constructs for the expression of these genes as well as a reporter gene (β-glucuronidase or green fluorescent protein) have been produced under the control of the three promoters, thus producing a total of 12 constructs. Transgenic lines were obtained by transformation of Arabidopsis plants with the corresponding constructs by floral dipping. With the exception of pyk10:At1g47960, all transformation experiments gave raise to independent transgenic lines. Corresponding control transgenic plants were, in addition, produced by transformation of wild type plants with the corresponding empty plasmids (pBINHygTX, for root specific expression, and pER8, for steroid inducible expression), although they were not used in the characterizations described here. Instead, wild type Col0 Arabidopsis plants were used for comparison with our transgenic plants.

### Plasmid Construction

Putative cell wall (At5g46940) and vacuolar (At1g47960) invertase inhibitors were initially cloned in pBluescript SK+between Acc65I and XbaI site. The corresponding PCR products, obtained by use of specific primers containing restriction sites for the subsequent cloning steps (Acc65I and XhoI for the 5′ primer and ApaI and XbaI for the 3′ primer), were digested with Acc65I and XbaI and cloned into pBluescript giving rise to pmcg2 and pmcg3 (FIG. 15) respectively (plasmid data sheets (PDS) MCG-4 (FIG. 16) and 5 (FIG. 17)).

The following primers were used:

Atcwinh-1:
5′-ctgaggtacctcgagcctgaaatggcttcttctc-3′
Atcwinh-2:
5-ctgatctagagggccctcattcaacaaggcgatc-3′
Atvinh-1:
5′-ctgaggtaccctcgagaagatgaagatgatgaagg-3′
Atvinh-2:
5′-gatctctagagggccctcaaagcaacattctcac-3′

More specifically, and referring to FIG. 16, the cDNA coding for an Arabidopsis thaliana cell wall invertase inhibitor (At5g46940) was amplified from RNA isolated from leaves by use of the primers Atcwinh-1 (5′-CTAGGGTACCTCGAGCCTGAAATGGCTTCTTCTC-3′), and Atcwinh-2 (5′-CTGATCTAGAGGGCCCTCATTCAACAAGGCGATC-3′), that generated Acc65I/XhoI and XbaI/ApaI restriction sites at the 5′ and 3′ end respectively. The generated product was cut with Acc65I and XbaI and cloned between the corresponding sites of the cloning plasmid pBluescript KS(+), generating the plasmid pmcg2.

Furthermore, and referring to FIG. 17, the cDNA coding for an Arabidopsis thaliana vacuolar invertase inhibitor (At1g47960) was amplified from RNA isolated from leaves by use of the primers Atvinh-1 (5′-CTGAGGTACCTCGAGAAGATGAAGATGATGAAGGT-3′), and Atvinh-2 (5′-GATCTCTAGAGGGCCCTCAAAGCAACATTCTCAC-3′), that generated Acc65I/XhoI and XbaI/ApaI restriction sites at the 5′ and 3′ end respectively. The generated product was cut with Acc65I and XbaI and cloned between the corresponding sites of the cloning plasmid pBluescript KS(+), generating the plasmid pmcg3.

First constructs for a root-specific expression of the invertase inhibitor used the root-specific pyk10 promoter of an Arabidopsis myrosinase. Pyk10 promoter was first amplified by PCR from genomic DNA, isolated from Arabidopsis leaves, with the primers pyk10-FORW and pyk10-REV. The product of this first PCR reaction was used for a nested PCR with pyk10-C and pyk10-F2 primers, designed as in Nitz et al. (2001), containing Acc65I restriction sites. The final PCR product was restricted with Acc65I and cloned into pTF2-6 (Nicotiana tabacum cell wall invertase inhibitor in pBINHygTx), giving rise to pmb1 (PDS MCG-3 (FIG. 18)). The following primers were used:

| | |
|---|---|
| pyk10-FORW: | 5′-gatgtacacgttttggtgtggg-3′ |
| pyk10-REV: | 5′-gcttacgtgtttagggaaatgg-3′ |
| pyk10-C: | 5′-ggacggtaccctgcaacgaagtgtacc-3′ |
| pyk10-F2: | 5′-gcaggtaccgtaattctgattttattcaag-3′ |

More specifically, and referring to FIG. 18, a construct for root specific expression of Nicotiana tabacum cell wall invertase inhibitor was generated in two steps. The promoter of pyk10 gene (AJ292756; Nitz et al., 2001), coding for a root specific myrosinase, was amplified by two sequential PCR reactions. In the first one, the primers pyk10-FORW (5′-GATGTACACGTTTTGGTGTGGG-3′) and pyk10-REV (5′-GCTTACGTGTTTAGGGAAATGG-3′) were used for amplification from genomic DNA isolated from Arabidopsis thaliana leaves. The product of this reaction was used in a nested PCR with the primers pyk10-C (5′-GGACGGTACCCTGCAACGAAGTGTACC-3′) and pyk10-F2 (5′-GCAGGTACCGTAATTCTGATTTTATTCAAG-3′), both containing an Acc65I restriction site. The product of this PCR reaction was cut with Acc65I and cloned into the corresponding site of plasmid pTF2-6, which corresponded to a cell wall invertase inhibitor (Y12805) cloned in the binary plasmid pBINHygTx (Gatz and Lenk, 1998) between Acc65I and XbaI restriction sites. The right orientation of the promoter in pmb1 was checked by restriction with different enzymes.

For the expression of the invertase inhibitors under control of the pyk10 promoter, At5g46940 and At1g47960 were cut from pmcg2 and pmcg3 respectively and cloned into pTF2-6 by restriction with Acc65I and XbaI, giving rise to pmcg4 (FIG. 19) and pmcg5 (FIG. 20) (PDS MCG-6 (FIG. 21) and MCG-7 (FIG. 22)). The promoter was inserted in front of the genes in both constructs by digestion of pmb1 with Acc65I, isolation of the pyk10 promoter fragment and cloning into Acc65I restricted pmcg4 and pmcg5, producing pmcg6 (FIG. 23) and pmcg7 (FIG. 24) (PDS MCG-8 (FIG. 25) and MCG-9 (FIG. 26)).

More specifically, and referring to FIG. 21, the cDNA coding for an Arabidopsis thaliana cell wall invertase inhibitor (At5g46940) was cut from pmcg2 plasmid by restriction with Acc65I and XbaI, and cloned between the corresponding sites of pTF2-6. For this, pTF2-6 plasmid was first cut with these enzymes and the band corresponding to the pBINHygTx binary plasmid isolated. Plasmid pmcg4, corresponding to a cell wall invertase inhibitor cDNA from Arabidopsis thaliana in pBINHygTx was generated.

More specifically, and referring to FIG. 22, the cDNA coding for an Arabidopsis thaliana vacuolar invertase inhibitor (At1g47960) was cut from pmcg3 plasmid by restriction with Acc65I and XbaI, and cloned between the corresponding sites of pTF2-6. For this, pTF2-6 plasmid was first cut with these enzymes and the band corresponding to the pBINHygTx binary plasmid isolated. Plasmid pmcg5, corresponding to a vacuolar invertase inhibitor cDNA from Arabidopsis thaliana in pBINHygTx was generated.

More specifically, and referring to FIG. 25, the construct for the root specific expression of an Arabidopsis thaliana cell wall invertase inhibitor (At5g46940) in the binary plasmid pBINHygTx was generated in two steps. First pyk10 promoter was cut from pmb1 with Acc65I and isolated from an agarose gel. The promoter was cloned between the corresponding sites of pmcg4, to generate pmcg6.

More specifically, and referring to FIG. 26, a construct for the root specific expression of an Arabidopsis thaliana vacuolar invertase inhibitor (At1g47960) in the binary plasmid pBINHygTx was generated in two steps. First pyk10 promoter was cut from pmb1 with Acc65I and isolated from an agarose gel. The promoter was cloned between the corresponding sites of pmcg5, to generate pmcg7.

The cryptic promoter was amplified by PCR from plasmid X7-KS, provided by Mollier et al. (2000), by use of cryp-F/R primers, containing an Acc65I restriction site on both ends. The PCR fragment was cloned into pmcg6-1 (pyk10:At5g56940 in pBINHygTx plasmid) giving rise to pmcg11 (FIG. 27) (PDS MCG-13 (FIG. 28)). For the generation of a fusion of cryptic promoter to At 1 g47960, the above described PCR fragment for the promoter was restricted with Acc65I and cloned into pmcg7-5 (pyk10:At1g47960 in pBINHygTx) giving rise to pmcg12 (FIG. 29) (PDS MCG-14 (FIG. 30)). In both plasmids pyk10 promoter present in pmcg6 and pmcg7 was removed by restriction with Acc65I and isolation of the plasmid band.

The following primers were used:

cryp-F: 5′-gatcggtacctcgaattgtgatatattgtaagc-3′
cryp-R: 5′-catggggtaccctgattaattagcaattagtgg-3′

More specifically, and referring to FIG. 28, a construct for root specific expression of an Arabidopsis thaliana cell wall invertase inhibitor (At5g46940) was generated in two steps. The promoter of a root specific cryptic gene (AX063411) was amplified by PCR by using the primers cryp-F (5′-GATCGGTACCTCGAATTGTGATATATTGTAAGC-3′) and cryp-R (5′-CATGGGGTACCCTGATTAATTAGCAATTAGTGG-3′), both containing an Acc65I restriction site, from the plasmid X7-KS provided by Mollier et al. (2000). The product of this PCR reaction was cut with Acc65I and cloned into the corresponding site of pmcg6. pmcg6 was first cut and the band corresponding to the plasmid pBINHygTx containing the cell wall invertase inhibitor isolated from an agarose gel. The right orientation of the promoter in pmcg 11 was checked by restriction with different enzymes.

More specifically, and referring to FIG. 30, a control construct for a strogen inducible expression of the reporter green fluorescent protein (GFP) was obtained from Dirk Becker (Lehrstuhl für Pflanzenphysiologie und Pflanzenbiophysik, Universität Würzburg) and transformed into Agrobacterium to use it for plant transformation.

The corresponding constructs in an estrogen-inducible system, pmcg8 (FIG. 31) and pmcg9 (PDS MCG-10 (FIG. 32) and MCG-11 (FIG. 33)) were obtained by restriction of pER8 plasmid and pmcg2 or pmcg3 with XhoI and ApaI and ligation of the digested plasmid to the isolated fragment corresponding to the gene.

More specifically, and referring to FIG. 32, a construct for a strogen inducible expression of an Arabidopsis thaliana cell wall invertase inhibitor (At5g46940) was generated by isolation of the corresponding cDNA from pmcg2, by restriction with XhoI and ApaI, and cloning between the corresponding sites of binary plasmid pER8.

More specifically, and referring to FIG. 33, a construct for an estrogen regulated expression of an Arabidopsis thaliana vacuolar invertase inhibitor (At1g47960) was generated by isolation of the corresponding cDNA from pmcg3, by restriction with XhoI and ApaI, and cloning between the corresponding sites of binary plasmid pER8.

See also Molier et al. (Mollier et al. (2000). Tagging of a cryptic promoter that confers root-specific gus expression in Arabidopsis thaliana. Plant Cell Rep. 19, 1076-1083.); Nitz I. et al. (Nitz I., Berkefeld H., Puzio P. S. and Grundler F. M. W. (2001). Pyk10, a seedling and root specific gene and promoter from Arabidopsis thaliana. Plant Sci. 161, 337-346.); and Zuo J. et al. (Zuo J., Niu Q. W. and Chua N. H. (2000). An estrogen receptor-based transactivator XVE mediates highly inducible gene expression in transgenic plants. Plant J. 24, 265-273.)

### EXAMPLE 2

### Growth of Transgenic Plants in Culture Medium Containing 1% Sucrose

Initial characterisation of the transgenic lines generated as described in example 1, was done with plants grown in Weck-glasses containing MS medium plus vitamins, MES, sucrose 1% and gelrite 0.3% for polymerisation. The seedlings were pre-grown first in selection medium with the same composition, but containing glucose 1% instead of sucrose and including hygromicin 50 mg/L for selection of transgenics but not for wild type control plants. After approximately 14 days of growth, seedlings were transferred to the culture medium described above.

More specifically, Arabidopsis seeds were pre-grown in MS0222 medium containing 0.5 g/L MES, 1% glucose and 0.3% gelrite, and including hygromicin in case of selection for transgenic seedlings. After 14 days, seedlings were transferred to Weck-glasses containing a similar medium but with sucrose 1% instead of glucose and without antibiotic. Shoot and root fresh weight were quantified and shoot-to-root ratio determined for 6 plants of each independent line. WTCol0, wild type Arabidopsis; AT4-11, pyk10:At5g46940; AT10-3 and 20, cryptic:At5g46940; AT11-9A, cryptic:At1g47960; AT15, pyk10:Cin1. For the Northern blot experiments, RNA was extracted from roots of each individual plant independently.

The quantification of shoot and root fresh weight in the plant showed that, interestingly, inhibition of invertase activity in the root did not result in a reduced root weight but in an unchanged or slightly increased weight, accompanied by a more marked increase in shoot weight mainly due to an increase in number of rosette leaves and shoots. Therefore, shoot-to-root ratio and whole plant biomass was increased in the invertase inhibitor plants. The effect of invertase inhibitor expression on shoot-to-root ratio and plant phenotype was more evident in case of cryptic promoter-driven expression as depicted in FIG. 3, even though gene expression was clearly more enhanced with the pyk10 promoter as depicted in FIG. 4.

More specifically, FIG. 3 represents the normal distribution of shoot-to-root ratio in WT plants and the different transgenic lines, FIG. 4 shows RNA expression, determined by Northern blot, of the corresponding invertase inhibitors in roots of transgenic plants in comparison to control WT plants, and FIG. 5 shows RNA expression of Cin1 in roots of transgenic plants.

The increase of invertase activity in the root by the tissue specific expression of an invertase gene (Cin1) resulted in a high variability of growth between plants of the same transgenic line, not rendering a clear phenotype in comparison to wild type plants. However, in plants grown in the culture medium a difference in phenotype in comparison to control plants was observed, with an increased root (0.33±0.21 respect to 0.19±0.08) and shoot (1.50±0.50 respect to 1.00±0.25) biomass to a similar extent, thus not producing a big difference in shoot-to-root ratio respect to control plants (FIG. 3). In this growth condition, there was also some degree of variability in the phenotype of transgenic plants, some of them showing abnormal proliferation of undifferentiated callus-like green structures instead of differentiated leaves and not flowering, while others flowered and presented an increased number of differentiated leaves (not shown). These structures appear also in some of the invertase inhibitor plants, although proper leaves and inflorescences developed in these plants as well. Impaired proper organ formation, leading to the appearance of poorly differentiated green structures, has been previously described in invertase antisense carrot embryos grown on sucrose-containing media. This effect was attributed to the missignaling produced by the altered sucrose-to-hexose level in the plantlets, since it could be compensated by the addition of glucose and fructose, the products of invertase reaction (Tang et al., 1999). When pyk10:Cin1 plants were grown in different conditions (soil or perlite) a higher variability in phenotype of plants was observed. In some cases plants presented an increased shoot-to-root ratio, whereas other plants of the same line showed a decreased ratio. As a result, the standard deviation calculated for the shoot to root ratio of the independent plants in a single characterisation experiment was in some cases as high as the medium value. This variation could be attributable to a degradation of the corresponding messenger in the plant, as observed by Northern blot studies for plants grown in culture medium (FIG. 5).

### EXAMPLE 3

### Growth of Transgenic Plants in Perlite

In a second characterisation experiment, plants were grown in perlite and watered with Hoagland's solution during 36 days in long day conditions (16 hours light/8 hours darkness), after the initial pre-growth in selection plates. Root and shoot fresh weight and shoot-to-root ratio, as well as seed weight per plant were determined for the different lines under study.

More specifically, Arabidopsis seeds were pre-grown in selection medium for 14 days and then transferred to perlite and grown for 36 days in LD conditions. Shoot and root fresh weight were quantified and shoot-to-root ratio determined for 8 plants of each independent line. WTCol0, wild type Arabidopsis; AT4-11, pyk10:At5g46940; AT10-3, 6.2 and 20, cryptic:At5g46940; AT11-3, 7 and 9A, cryptic:At1g47960; AT15, pyk10:Cin1.

The results are shown in FIG. 6.

As may be taken from FIG. 6, plants of pyk10:Cin1 transgenic line behaved in a different way in respect to the previous experiment. Shoot-to-root ratio was increased in these plants, root fresh weight was decreased (0.017±0.007 respect to 0.038±0.027 in wild-type) and shoot fresh weight slightly increased (0.20±0.09 respect to 0.16±0.07 in wild-type) respect to control plants. For the invertase inhibitor, pyk10-driven expression of invertase inhibitor resulted in a clearer tendency to an increased shoot-to-root ratio than in culture medium, although the increase was again clearer in lines where the expression of the invertase inhibitor was under control of the cryptic promoter (FIG. 6).

The increase of this ratio was mainly due to an increased shoot fresh weight in these lines, as a result of an increased shoot number, whereas root mass was only increased in one of the lines (AT11-3) analysed for cryptic: At1g47960, but not in the other independent lines for the same construct. Interestingly, the analysis of seed weight per plant showed also an important difference between seed yield of transgenic plants and wild types. Seed yield was clearly increased in the different invertase inhibitor lines, whereas in the pyk10:Cin1 line it was slightly decreased respect to control despite the slight increase in shoot biomass (Table 3).

TABLE 3
Duncan's multiple range test for medium values of plants: Plants
are grouped into significance groups designated by a letter. Lines
with the same letter are not significantly different.

| | | | | Seed weight |
|---|---|---|---|---|
| Line | Root FW (g) | Shoot FW(g) | Shoot/root | (mg)/plant |
| WTCol0 | 0.038750 bcd | 0.158750 d | 4.722500 e | 4.712500 ef |
| AT15 | 0.016875 d | 0.203750 d | 12.271250 bc | 2.462500 f |
| AT4-11 | 0.063750 b | 0.630000 bc | 10.526251 cd | 13.425000 bcd |
| AT10-3 | 0.056250 bc | 0.671250 b | 12.902500 bc | 17.562500 b |
| AT10-6.2 | 0.046250 bcd | 0.337500 d | 7.753750 de | 12.049999 cd |
| AT10-20 | 0.026875 cd | 0.408750 cd | 15.785000 ab | 16.600000 bc |
| AT11-3 | 0.108750 a | 1.318750 a | 13.961250 abc | 8.705000 de |
| AT11-7 | 0.044375 bcd | 0.755000 b | 17.344999 a | 27.634998 a |
| AT11-9A | 0.062500 b | 0.778750 b | 13.515000 abc | 23.212500 a |
| Coef. Var. | 57.292% | 39.766% | 32.203% | 35.891% |

The clearest phenotype, in respect to seed yield and shoot-to-root ratio, was obtained in those lines expressing AtC/VIF1 (At1g47960) under the control of the cryptic promoter. Therefore these lines, designated as AT11, were used for a detailed characterisation. Two independent lines for this construct (AT11-7 and AT11-9), that contained different copy number and insertion sites, as shown by Southern blot experiments (FIG. 7), were used for the detailed characterisations.

For preparing the Southern blot of three independent lines for AT11 (cryptic:At1g47960), 2 μg of gDNA of each line were digested with BamHI (B), EcoRI (E), HindIII (H) and XbaI (X) restriction enzymes that do not cut inside the probe sequence. Digested DNA was analysed by electrophoresis in 0.7% agarose gel and blotted onto a nitrocellulose membrane. Filter was hybridised with a radioactive labelled probe corresponding to the HPTII gene.

As a final characterisation experiment, plants were grown in the previously described conditions, in perlite during approximately 49 days in long day conditions (16 hours light/8 hours darkness). In this case, the pre-growth was done in selection medium containing sucrose 1% instead of glucose, to study the influence of the sugar in this medium on subsequent plant growth. Growth time was increased from 36 to 49 days to analyse if the differences between lines are clearer after longer growth periods. Again, root and shoot fresh weight and shoot-to-root ratio were determined for the different lines under study, but in this case the values for seed production correspond to silique dry weight. The main difference between this experiment and the preceding one is that AT15 (pyk10:Cin1) plants behave more similar to wild types, with a shoot-to-root ratio more similar to wild type than in the previous experiment (FIG. 8). Again, the increase of this ratio in invertase inhibitor lines was due to an increased shoot fresh weight. Only a reduction in root fresh weight respect to control plants was observed in AT10-20 (cryptic:At5g4694), accompanied by a slightly reduced shoot weight respect to wild type plants, but with an increased shoot-to-root ratio. In all other lines analysed, shoot-to-root ratio was increased respect to control, although for AT15 the differences with WTCol0 were not statistically significant according to Duncan's test (Table 4).

For such purpose, Arabidopsis seeds were pre-grown as described before, except that glucose was substituted by sucrose 1%, and then transferred to perlite and grown for 49 days in LD conditions. Shoot and root fresh weight were quantified and shoot-to-root ratio determined for 7 plants of each independent line. WTCol0, wild type Arabidopsis; AT4-11, pyk10:At5g46940; AT10-6.2 and 20, cryptic:At5g46940; AT11-7 and 9A, cryptic:At1g47960; AT15, pyk10:Cin1.

In respect to seed yield, determined as silique dry weight per plant, invertase inhibitor lines showed a significant increase of this parameter. In this case, AT15 plants (pyk10:Cin1) showed a slight, but not statistically significant, increase of seed yield respect to wild type. The most pronounced phenotypic difference was again observed for the cryptic promoter-driven invertase inhibitor expression, whereas differences to control were not so pronounced with the pyk10 promoter. Lines AT11-7 and AT11-9A were again the lines showing the more significant difference, in shoot-to-root ratio and seed production, respect to controls.

TABLE 4
Duncan's multiple range test for medium values of plants: Plants
are grouped into significance groups designated by a letter. Lines
with the same letter are not significantly different.

| | | | | Silique DW |
|---|---|---|---|---|
| Line | Root FW (g) | Shoot FW(g) | Shoot/root | (mg)/plant |
| WTCol0 | 0.137143 ab | 0.980429 cd | 6.892000 c | 20.857143 c |
| AT15 | 0.204286 a | 2.257143 ab | 11.454286 bc | 45.571429 bc |
| AT4-11 | 0.101429 bc | 1.580000 bc | 15.611428 ab | 44.714286 bc |
| AT10-6.2 | 0.102857 bc | 1.547143 bc | 16.541429 ab | 66.571429 b |
| AT10-20 | 0.061429 c | 0.704286 d | 12.191428 b | 46.571429 bc |
| AT11-7 | 0.181429 a | 2.867143 a | 18.045714 a | 122.285714 a |
| AT11-9A | 0.147143 ab | 2.425714 a | 17.930000 a | 111.571429 a |
| Coef. Var. | 47.798% | 37.007% | 31.532% | 51.972% |

For such purpose, Arabidopsis seeds were pre-grown in selection medium containing sucrose for 14 days and then transferred to perlite and grown for 49 days in LD conditions. Shoot and root fresh weight were quantified and shoot-to-root ratio determined for 7 plants of each independent line. Silique weight per plant was determined once siliques were dry. WTCol0, wild type Arabidopsis; AT4-11, pyk10:At5g46940; AT10-6.2 and 20, cryptic:At5g46940; AT11-7 and 9A, cryptic:At1g47960; AT15, pyk10:Cin1.

As an example of the phenotype of the invertase inhibitor lines, photographs of AT11-7 and 9A are shown in FIG. 9, corresponding to the last characterisation experiment described. The pictures show that plants of the transgenic lines are bigger and have an increased number of leaves and shoots than the wild type plants. Although shoot weight is increased in plants of the transgenic lines, the increase in seed yield is not only due to the increased shoot mass but in addition silique number and consequently silique weight per shoot fresh weight is increased. As an example, in the experiment analysed in Table 4, silique DW (mg)/shoot FW(g) was increased from 21,28 in wild type plants to 42,76 and 46,10 in AT11-7 and AT11-9A respectively. For this reason, we determined in an independent experiment silique number per shoot fresh weight in wild type and transgenic plants pre-grown in glucose plates, our standard pre-growth conditions, and subsequently grown in perlite for 40 days. Results of Duncan's test for these two parameters are shown in Table 5.

TABLE 5
Duncan's multiple range test for medium values of plants: Plants
are grouped into significance groups designated by a letter. Lines
with the same letter are not significantly different.

| Line | Silique number/Shoot FW | Silique DW (mg)/Shoot FW (g) |
|---|---|---|
| WTCol0 | 38.215497 b | 19.277501 c |
| AT11-7 | 61.655353 a | 45.408005 a |
| AT11-9A | 54.039001 a | 32.318250 b |
| Coef. Var. | 27.16% | 43.15% |

For such purpose, Arabidopsis seeds were pre-grown in selection medium containing glucose for 14 days and then transferred to perlite and grown for 40 days in LD conditions. Silique number and dry weight were quantified, and silique number/shoot FW and silique DW/shoot FW determined for 20 plants of each independent line. Silique dry weight was determined once siliques were dry. WTCol0, wild type Arabidopsis; AT11-7 and 9A, cryptic:At1g47960; AT15, pyk10:Cin1.

In FIG. 9 the phenotype of independent plants of WTCol0, AT11-7 and AT11-9A. For such purpose, Arabidopsis plants were pre-grown in selection medium containing sucrose and transferred to perlite after 14 days. Photographs were obtained after 49 days of growth at LD conditions, at the moment of plant material collection.

### EXAMPLE 4

### Phenotype of Transgenic Plants in Other Growth Conditions

In order to study if the transgenic lines phenotype was associated to the particular growth conditions used in the characterisation experiments, plants were grown in different growth conditions and phenotype analysed in terms of shoot and root fresh weight, shoot-to-root ratio and seed yield. Plants of the two selected transgenic lines, AT11-7 and AT11-9A, and wild type Col0 plants were grown in LD conditions in soil, SD conditions in perlite and the previously described conditions, LD conditions in perlite, for comparison.

### 1. Growth of Plants in Perlite and Short Day Conditions

For the analysis of plant phenotype on short day conditions (8 h light/16 h darkness), seeds were pre-grown in selection medium with glucose for 14 days at LD conditions, and then transferred to perlite and SD conditions for 43 days. At this time shoot and root fresh weight and seed yield were measured. As shown in FIG. 10 (Photographs were obtained after 43 days of growth at SD conditions, at the moment of plant material collection. 10 plants of each line were used in this experiment). plants of the two transgenic lines under analysis are slightly bigger and have an increased number of leaves respect to wild types. In addition, a high percentage of the transgenic plants flowered (100% in AT11-7 and 70% of AT11-9A) and presented some siliques, whereas only 40% of the wild type plants flowered and presented a reduced number of siliques.

Statistic analysis was difficult to perform due to the high variability of silique number and fresh weight between plants of the same line. However shoot-to-root ratio was increased from 12.73±2.62 in wild types to 17.80±4.18 and 15.06±3.30 in AT11-7 and AT11-9A respectively. This increase was significant according to Duncan's test. Silique number per shoot fresh weight was 2.04±2.69 in wild types, 12.98±7.06 and 5.60±6.42 in AT11-7 and AT11-9A respectively, considering all plants. The high variability in wild type and AT11-9A was due to the presence of some plants that had not produced any silique at the time point analysed. The analysis of longer growth periods could result in a more clear difference in phenotype, revealing differences in seed yield between wild type and transgenics once all plants have flowered, and allowing a more reliable statistical analysis of the different parameters under study.

### 2. Growth of Plants in Soil And Long Day Conditions

Plants grown on soil in LD conditions (16 h light/8 h darkness) showed accelerated growth respect to perlite and therefore the growth time for the phenotypic analysis was reduced to 30 days. Transgenic plants showed reduced growth in comparison to perlite-grown plants, with a slightly reduced length respect to wild type plants and increased branching of shoots, but still a difference in shoot-to-root ratio respect to wild types was observed (FIG. 11: Phenotype plants of WTCol0, AT11-7 and AT11-9A. Arabidopsis plants were pre-grown in selection medium containing glucose and transferred to perlite after 14 days. Photographs were obtained after 30 days of growth at LD conditions, at the moment of plant material collection. 6 plants were analysed of each of the lines under study.). This difference was mainly due to a significantly reduced root weight respect to wild type plants and a non-significantly altered shoot weight (Table 6). Nevertheless, this data should be carefully taken into consideration, since root recovery from soil is difficult in comparison to perlite and some loss of material could take place during the root collection.

Analysis of data according to Duncan's test showed that although there was no significant variation of silique number or weight per plant between wild types and transgenics, silique number and fresh weight per gram of shoot fresh weight was significantly increased, being approximately two times the value obtained in wild type plants (Table 6).

TABLE 6
Duncan's multiple range test for medium values of plants:
Plants are grouped into significance groups designated by a letter.
Lines with the same letter are not significantly different.

| Line | Root FW (g) | Shoot FW (g) | Shoot/root | Silique n^{o}/Shoot FW | Silique FW (mg)/Shoot FW (g) |
|---|---|---|---|---|---|
| WTCol0 | 0.186000 a | 1.688333 a | 9.848333 b | 21.060000 b | 67.346670 b |
| AT11-7 | 0.075000 b | 1.310000 ab | 17.589999 a | 43.741664 a | 148.926666 a |
| AT11-9A | 0.065000 b | 0.948333 b | 16.048333 a | 45.190002 a | 158.349996 a |
| Coef. Var. | 42.75% | 28.58% | 34.61% | 33.90% | 36.07% |

For such purpose, Arabidopsis seeds were pre-grown in selection medium containing glucose for 14 days and then transferred to soil and grown for 30 days in LD conditions. Silique number and fresh weight were quantified, and silique number/shoot FW and silique FW/shoot FW determined for 6 plants of each independent line. Silique fresh weight was determined at the time of collection. WTCol0, wild type Arabidopsis; AT11-7 and 9A, cryptic:At1g47960.

This characterisation experiment points out that although size of transgenic plants is reduced in soil respect to wild type plants, instead of increased as observed in perlite, shoot-to-root ratio and seed production per shoot fresh weight are still increased.

### 3. Growth of Plants in Perlite and Long Day Conditions

As a comparison for growth, plants of WTCol0 and the two transgenic lines were grown as in previous experiments. Seedlings pre-grown in selection plates containing glucose were transferred to perlite and grown for additional 40 days. Shoot, root and silique fresh weight as well as silique number were determined and the corresponding parameters under analysis calculated (included in Table 7). For such purpose, Arabidopsis seeds were pre-grown in selection medium containing glucose for 14 days and then transferred to perlite and grown for 40 days in LD conditions. Shoot and root FW were measured in 20 plants of each independent line. Silique fresh weight was determined at the time of collection. WTCol0, wild type Arabidopsis; AT11-7 and 9A, cryptic:At1g47960. More specifically, Arabidopsis seeds were pre-grown in selection medium containing glucose for 14 days and then transferred to perlite and grown for 40 days in LD conditions. Silique number and fresh weight per plant were measured at the time of collection. WTCol0, wild type Arabidopsis; AT11-7 and 9A, cryptic:At1g47960.

TABLE 7
Duncan's multiple range test for medium values of plants: Plants are
grouped into significance groups designated by a letter. Lines with the
same letter are not significantly different.

| Line | Root FW (g) | Shoot FW(g) | Shoot/root | Silique n^{o}/Shoot FW | Silique FW(mg)/Shoot FW(g) |
|---|---|---|---|---|---|
| WTCol0 | 0.034980 a | 0.420710 b | 11.864001 b | 58.289508 b | 147.127991 b |
| AT11-7 | 0.036720 a | 0.666105 a | 18.538249 a | 96.629999 a | 257.697510 a |
| AT11-9A | 0.042080 a | 0.787255 a | 20.966499 a | 87.278503 a | 266.290015 a |
| Coef. Var. | 51.34% | 50.12% | 26.75% | 21.60% | 21.84% |

As observed in the previous characterisation experiments, shoot-to-root ratio was significantly increased in transgenic plants respect to wild types even though, as happened with the previous in soil-characterisation, this ratio was higher in wild type plants in this experiment than in previous characterisations with plants grown in perlite (Tables 3 and 4). Still the shoot-to-root ratio increase is due to the increased shoot fresh weight in transgenic plants, whereas root fresh weight was not significantly altered. Values of shoot weight in transgenic lines were comparable to that obtained in previous characterisation experiments in similar conditions (Table 3.1), but in the case of wild type plants an increase respect to other characterisations was produced resulting in the mentioned increase in shoot-to-root ratio. These results also confirm that overall growth of plants is increased in both wild type and transgenic lines when pre-growth is done in selection plates containing sucrose respect to glucose (see data in Table 3.4 in comparison to Table 3 and 7).

In respect to seed yield, a significant increase in silique number or fresh weight per shoot fresh weight respect to wild type plants was observed for the two independent transgenic lines under analysis (Table 7). The increases respect to wild type are proportional to those observed in a previous experiment done in the same conditions (see Table 5 for comparison), taking into consideration that in the previous experiment silique dry weight instead of fresh weight was determined. In that case, even though results were not shown, changes of silique fresh weight per shoot fresh weight were proportional to those shown for dry weight in Table 5

TABLE 8
Duncan's multiple range test for medium values of plants: Plants
are grouped into significance groups designated by a letter. Lines
with the same letter are not significantly different.

| Line | Silique n°/plant | Silique FW (mg)/plant |
|---|---|---|
| WTCol0 | 23.350000 b | 60.274994 b |
| AT11-7 | 61.600000 a | 187.224976 a |
| AT11-9A | 66.900000 a | 211.472485 a |
| Coef. Var. | 46.81% | 56.06% |

Silique number and fresh weight per plant are also increased respect to wild types in the actual experiment (Table 7) in difference to soil-grown plants, although the increase was smaller than in sucrose pre-grown plants. The phenotype of plants is shown in FIG. 12 (Phenotype plants of WTCol0, AT11-7 and AT11-9A after 40 days of growth at LD conditions. Arabidopsis plants were pre-grown in selection medium containing glucose and transferred to perlite after 14 days. Photographs were obtained after 40 days of growth at LD conditions.)

### EXAMPLE 5

### Determination of Invertase Inhibitor Activity in Roots of Transgenic Plants

As shown herein, Northern blot analysis demonstrated that effectively transgenic lines have an increased expression of the corresponding invertase inhibitor with respect to wild type plants. This increase reflects the accumulation of messenger RNA for the corresponding gene, but does not prove the activity of the enzyme on invertase inhibition. The determination of the inhibition of invertase activity in roots by the inhibitor in an “in vitro” assay presents the difficulty that the assay is generally performed in optimal conditions where the sugar concentration is not the actual concentration present in the root, besides the described protection effect of sucrose on invertase inhibition at a concentration lower than that used in the activity assay (Rausch and Greiner, 2004). In addition, the determination of invertase activity at a determined stage of growth may not necessarily show differences between transgenics and wild types, due to the spatio-temporal activity of the promoter used to direct gene expression. Instead, changes of invertase activity at early stages of development may alter plant growth and assimilate partitioning, resulting in the observed phenotypes. Moreover, Greiner et al. (1999) reported that the stability of the complexes formed between invertases and the inhibitors during preparation may depend on tissue specific factors. The analysis of vacuolar invertase activity in transgenic potato plants that ectopically expressed a tobacco invertase inhibitor showed a decreased activity in leaves but not in transgenic tubers (Greiner et al., 1999), even though levels of transcripts of invertase inhibitor were clearly increased in both organs in respect to control plants. This result was explained by the authors in base of a different stability of the complex in tubers in respect to leaves. For this reason, the determination of the protein levels of the invertase inhibitor by use of a specific antibody would be of interest. However, no good antibody is so far available. Therefore, we are now aiming to obtain antibodies against the invertase inhibitor by heterologous expression of the At1g47960 in E. coli.

As previously mentioned, of the two invertase inhibitors of Arabidopsis used in these studies AtC/VIF1 (At1g47960) inhibits specifically vacuolar invertase activity in in vitro assays, whereas AtC/VIF2 (At5g46940) inhibits both although with a higher affinity for vacuolar than for cell wall invertase (Link et al., 2004). However, so far intracellular localisation of these proteins has not been analysed. As an attempt to analyse the effect of invertase inhibitor on invertase activity in roots, we initially measured total invertase activity in root extracts of wild type and transgenic plants of the two selected lines, AT11-7 and AT11-9A (AtC/VIF1), and AT4-11 (AtC/VIF2). The results showed that there was no significant variation of cell wall invertase activity between roots of control and transgenic plants, not either for vacuolar invertase activity although with a slight increase in AtC/VIF1 invertase inhibitor plants (data not showed). As mentioned before, some dissociation of the complex formed between the invertase and the corresponding inhibitor could occur during preparation of the extracts and the stability of this complex could depend on tissue specific factors present in the root. In order to circumvent possible problems of complex stability during the isolation procedure, a mixed-extract assay was developed in which an aliquot of a root extract of a transgenic plant was mixed with an aliquot of a leaf extract of a wild type plant. This mix was done in a final volume of 570 μl in phosphate buffer pH 4.5, and incubated for 30 minutes at 37° C. for the formation of the complex between the invertase and the proteinaceous inhibitor. After the incubation, sucrose was added at a final concentration of 5 mM and the reaction incubated at 26° C. during 30 min, for invertase mediated degradation of sucrose. In this way, the addition of sucrose after the pre-incubation step should prevent possible sucrose protection effect on invertase inhibition (Weil et al., 1994; Greiner et al., 1998). After the incubation, reaction was stopped in ice and the glucose released measured by use of the GOD reagent (Roitsch et al., 1995). Invertase activity in the mix was compared with the added value of the leaf extract and root extract incubated separately. Although only preliminary tests have been done so far, results showed an invertase inhibitory effect of the transgenic root extracts. As an example, incubation of soluble fraction of a leaf extract with the soluble fraction of a root extract of At1g47960 lines resulted in 79% invertase activity respect to the added value. For At5g46940 soluble fraction, values in mixed extracts assay were 86% of the added value of the corresponding extracts. In a mixed assay with a soluble protein extract of wild type roots value of invertase activity was 123% of the added value. The results suggest that effectively invertase inhibitor activity is increased in soluble fraction of transgenic roots. But so far, due to the limitation of material available, no tests have been done with the cell wall fraction of transgenic root material.

These results suggested that the mixed extracts assay could be a way to determine invertase inhibitor activity in roots of transgenic lines. However, the limitation of root material available made it difficult to improve conditions in the assay. As a tool for establishing the assay, leaves of transgenic tobacco plants expressing an apoplasmic invertase inhibitor (Greiner et al., 1998) under control of a cytokinin inducible promoter (Lin6), used in the previously described research project on invertase role on cytokinin-mediated delay of senescence (Balibrea-Lara et al., 2004), were used. Leaves were infiltrated with a MS-Silwet solution containing kinetin (30 μg/L) and proteins were extracted 3 h after infiltration. The invertase inhibitor expressed in these plants has been shown to be localised in the cell wall (Weil et al., 1994), therefore insoluble protein fraction was used in the assays. Soluble fraction was used as a control where no effect on added values of independently incubated extracts should be observed. The availability of bigger quantities of material in this case allowed concentration of proteins through a Centricon 10K column, with a concentration factor of 11.6 and 21.7 in the soluble and insoluble fraction of the treated leaves respectively. Mixed extracts of wild type leaves cell wall fraction and concentrated cell wall fraction of infiltrated transgenic leaves showed 70% invertase activity respect to added value of corresponding independent reactions (considered as 100%). Similar reduction was observed with a non-concentrated cell wall fraction of infiltrated transgenic leaves, but with a 4:1 ratio of volume of transgenic insoluble fraction respect to wild type soluble fraction. No differences respect to added value were obtained when soluble fractions of transgenic leaves were used in combination with the cell wall fraction of wild type leaves, in accordance with the localisation of the transgene in the cell wall fraction. So far, the concentration of sucrose in the assay for invertase activity was 5 mM, far above the Km of invertases for sucrose, in order to measure maximum invertase activity. We have performed the assays in presence of a smaller concentration of sucrose, 1 mM, in order to see if the effect of the inhibitor on invertase activity is more clearly detected. The invertase activity of a cell wall fraction of wild type leaves combined with a concentrated cell wall fraction of transgenic leaves was 58% of the added value, whereas no differences to added values were observed for a concentrated soluble fraction of transgenic leaves. When a non-concentrated cell wall transgenic extract was used, values in the mixed assay were from 55 to 72% of the added value. Mixed extracts with a soluble concentrated/non-concentrated fraction of transgenic leaves, in combination with cell wall fraction of wild type leaves, showed no difference in invertase activity respect to the added values. These results suggest that this method could be suitable for the determination of invertase inhibition by invertase inhibitor. Invertase inhibitor activity will be evaluated in the different transgenic lines by use of mixed extracts, thus allowing not only the determination of the inhibition of invertase activity but also the localisation of the protein to the cell wall or soluble fraction, although the results obtained so far indicate the presence of inhibitory activity in the soluble fraction for both invertase inhibitors used in the transgenics.

### EXAMPLE 6

### Increased Resistance of Roots of Transgenic Arabidopsis thaliana Against Infection by Plasmodiophora brassicae

Transgenic A. thaliana plants expressing

a) pyk10-Promoter:AtC/VIF2; or
b) crypticT80: AtC/VIF2
were generated in accordance with the experimental procedure outlined in example 1.

Resistance of the roots of the two A. thaliana strains were tested as follows:

Fourteen-day-old plants cultivated under greenhouse conditions or under controlled environment (21° C., 16 h light, 100 μmol photons/s/m²) were routinely inoculated by injecting the soil around each plant with 2 ml of a resting spore suspension of the pathogen with a standard concentration of 10⁶ spores/ml according to Fuchs and Sacristan (1996). Other time points of inoculation were chosen for the respective experiments and are given in the results section.

Disease symptoms were assessed 28 days after inoculation (dai) using a scale consisting of five classes according to Klewer et al. (2001): 0 (no symptoms), 1 (very small clubs, mainly on lateral roots that do not impair the main root), 2 (small clubs covering the main root and few lateral roots), 3 (medium sized to bigger clubs, also including the main root, plant growth might be impaired), 4 (severe clubs in lateral, main root or rosette, fine roots completely destroyed, plant growth is affected). Disease index (DI) was calculated using the five-grade scale according to the formula: DI=(1·n₁+2·n₂+3·n₃+4·n₄)·100/4·Nt, where n₁ to n₄ is the number of plants in the indicated class and Nt is the total number of plants tested.

The results are depicted in FIGS. 34 and 35. As may be taken from FIG. 34, the roots of transgenic plants infected by Plasmodiophora brassicae showed an increase in resistance.

In contrast, single KO lines were not significantly affected as depicted in FIG. 35.

### EXAMPLE 7

### Infection of Roots of Tobacco (Nicotiana tabacum) by the Mycorhiza Fungus Glomus intraradices: Effect on Mycorrhization

Arbuscular mycorrhiza (AM) represents a widespread mutualistic association between soil-born fungi of the phylum Glomeromycota and most land plants.

We have studied the impact of a reduced hexose availability on mycorrhization of N. tabacum or M. truncatula. Decreased root hexose content was achieved by root-specific expression of A. thaliana invertase inhibitor. By specifically inhibiting root invertase activity the requirement of sufficient hexose supply for AM growth could be documented. N. tabacum pyk10::InvInh plants with decreased acid invertase activity in roots exhibited a diminished mycorrhization. Insofar this sample provides for another piece of evidence that the interaction between microorganisms and plants may be affected in a negative manner. Based on this observation also the interaction between plants and plant pathogens such as Plasmodiophora can be affected and thus a strategy for the protection of plants based on inhibition of invertase activity is provided.

### Plasmid Constructions, Stable Plant Transformation and Determination of Plant Invertase Activities

The pyk10 promoter was amplified by PCR using genomic DNA and subcloned into the vector pTF2-6 (T. Fatima and T. Roitsch, unpublished) to generate pMB1-18. The cDNA encoding AtC/VIF2 (at5g64620) was amplified by RT-PCR using total RNA, initially cloned into the vector pBluescript KS+ to generate pMCG2, and subsequently subcloned thereof as Acc65I-KpnI fragment into the binary vector pTF2-6 to generate plasmid pMCG4. To generate a transcriptional fusion between the pyk10 promoter and the cDNA encoding AtC/VIF2, a 1467 bp pyk10 promoter fragment was subcloned as Acc65I fragment from pMB1-18 into the binary vector pMCG4, linearized by Acc65I, to generated pMCG6. The pyk10::InvInh construct was transformed in tobacco (Nicotiana tabacum cv. SR1) using Agrobacterium tumefaciens strain LBA4404 and standard transformation procedures (Horsch et al. 1985). Transgenic lines expressing the pyk10::InvInh fusion were characterized by PCR (M. Gonzalez and T. Roitsch, unpublished).

The results of this kind of experiments are depicted in FIG. 36 to 38. More specifically, FIG. 36 shows the result of an analysis of transgenic tobacco plants with root-specific expression of an invertase inhibitor. A and B, Cell wall (A) and vacuolar (B) invertase activity in roots of wild-type SR1 plants and NT pyk10::InvInh plants of two independent lines (98-1-10 and 98-4-1) 3.5 and 5 weeks after inoculation with G. intraradices. C, Glucose and fructose content of the roots. D, Ratio of the glucose and fructose contents to the sucrose content of the roots. E, Degree of mycorrhization. To allow statistical analysis, the degree of mycorrhization in percent of the root length was determined for every root system in 50 to 100 root pieces of each 1 cm length. Plants were in two independent experiments inoculated with G. intraradices either 2.5 weeks after sowing and harvested 3.5 weeks later or inoculated 4 weeks after sowing and harvested 5 weeks later. Data are presented as mean values+SD (at 3.5 weeks: n=5; at 5 weeks: n=3). The data from the transgenic lines were pairwise compared to the wild-type by the Student t test. *P<0.05, **P<0.01. F, Ink-stained fungal structures in a wild-type and a NT pyk10::InvInh plant of line 98-1-10, each 5 weeks after inoculation. Bars represent 100 μm.

FIG. 37 shows the result of a biomass analysis of NTpyk10::InvInh plants. Root-to-shoot ratio of the fresh weight of 6-week-old non-mycorrhizal wild-type SR1 plants and plants of two independent NT pyk10::InvInh lines (98-1-10 and 98-4-1). Mean values of +SD are given. (n≧33).

FIG. 38 shows the result of determining invertase activities in non-mycorrhizal and mycorrhizal NT pyk10::Inylnh plants. A, Cell wall bound invertase activities in roots. B, Vacuolar invertase activities in roots. C, Cytosolic invertase activities in roots. D, Apoplastic invertase activities in leaves. Wild-type SR1 plants and plants of the two NT pyk10::InvInh lines 98-1-10 and 98-4-1 were inoculated with G. intraradices 4 weeks after sowing and harvested 3 and 5 weeks later. Data are presented as mean values+SD (n=3). The data from the non-mycorrhizal and mycorrhizal transgenic plants were compared to the non-mycorrhizal and mycorrhizal wild-type plants, respectively, using the Student t test. *P<0.05, **P<0.01.

As confirmed and illustrated, respectively, by the results indicated in FIGS. 36 to 38, because a general undersupply of the root with carbon by defective phloem loading resulted in decreased mycorrhization, the analysis of plants with reduced invertase activity and decreased phloem unloading complement this study. This aspect was implemented by expressing the Arabidopsis gene AtC/VIF2 coding for an inhibitor of acid invertases (Link et al., 2004) under control of the root- and seedling-specific pyk10 promoter from Arabidopsis (Nitz et al., 2001) in transgenic N. tabacum plants (NT pyk10::InvInh). Recombinant AtC/VIF2 protein was shown to affect apoplastic and vacuolar invertase activities in vitro (Link et al, 2004). Plants of the two independent NT pyk10::InvInh lines, 98-1-10 and 98-4-1, showed reduced apoplastic invertase activities in the root (FIG. 36 A). Vacuolar invertase activity was inhibited in vitro only in one line at later developmental stages (FIG. 36 B). Neutral cytosolic invertase activity levels were not affected; the same was true for invertases in leaves (FIG. 38). Non-mycorrhizal plants showed a similar affection of invertase activities (FIG. 38). According to the reduced apoplastic invertase activity, the roots had lower contents of glucose and fructose (FIG. 36 C shows the sum of both hexoses) and a reduced ratio of both hexoses to sucrose (FIG. 36 D). However, in contrast to rolC::ppa tobacco, plants with root-specific overexpression of invertase inhibitor were not altered in their vegetative growth and their root or shoot biomass compared to wild-type plants, whereby FIG. 37 shows the root-to-shoot ratio of the fresh weight as determined in principle, as described in the example part herein. Nevertheless, corresponding to the lower hexose levels in roots of pyk10::InvInh plants we found a lower mycorrhization level with G. intraradices (FIG. 36 E). Moreover, colonized roots of NT pyk10::InvInh plants showed a lower density of fungal structures compared to the wild-type (FIG. 36 F) reflected by a significant decrease in fungus-specific rRNA 5 weeks after inoculation (data not shown). This indicates that the carbon supply in the AM interaction depends on the activity of apoplastic invertases that deliver hexoses.

### REFERENCES

Throughout the present specification the following references were recited the disclosure of which is herein incorporated by reference in their entirety.
Balibrea Lara M. E., Gonzalez Garcia M.-C., Fatima T., Ehness R., Lee T. K., Proles R., Tanner W. and Roitsch T. (2004). Extracellular invertase is an essential component of cytokinin-mediated delay of senescence. Plant Cell 16, 1276-1287.
Bret-Harte M. and Silk W. K. (1994). Nonvascular, symplasmic diffusion of sucrose cannot satisfy the carbon demands of growth in the primary root tip of Zea mays L. Plant Physiol. 105, 19-33.
Eschrich W. (1980). Free space invertase, its possible role in phloem unloading. Ber. Deutsch. Bot. Ges. Bd. 93, S363-378.
Farrar J. F. and Jones D. L. (2000). The control of carbon acquisition by roots. New Phytol. 147, 43-53.
Fuchs, H., M. D. Sacristan (1996): Identification of a gene in Arabidopsis thaliana controlling resistance to clubroot (Plasmodiophora brassicae) and characterization of the resistance response. Mol. Plant. Microbe Interact. 9, 91-97.
Gatz C, Lenk I (1998) Promoters that respond to chemical inducers. Trends in Plant Science 3, 352-358.
Goetz M., Godt D. E., Guivarc'h A., Kahmann U., Chriqui D. and Roitsch T. (2001). Induction of male sterility in plants by metabolic engineering of the carbohydrate supply. Proc. Natl. Acad. Sci. U.S.A. 98, 6522-6527.
Greiner S., Krausgrill S. and Rausch T. (1998). Cloning of a tobacco apoplasmic invertase inhibitor: Proof of function of the recombinant protein and expression analysis during plant development. Plant Physiol. 116, 733-742.
Greiner S., Rausch T., Sonnewald U. and Herbers K. (1999). Ectopic expression of a tobacco invertase inhibitor homolog prevents cold-induced sweetening of potato tubers. Nature Biotech. 17, 708-711.
Heyer A. G., Raap M., Schroeer B., Marty B. and Willmitzer L. (2004). Cell wall invertase expression at the apical meristem alters floral, architectural, and reproductive traits in Arabidopsis thaliana. Plant J. 39, 161-169.
Klewer A., H. LuerBen, H. Graf, J. Siemens (2001): RFLP markers to characterise Plasmodiophora brassicae single-spore-isolates with different virulence patterns. J. Phytopathol. 149, 1-7.
Link M, Rausch T, Greiner S (2004) In Arabidopsis thaliana, the invertase inhibitors AtC/VIF1 and 2 exhibit distinct target enzyme specificities and expression profiles. FEBS Lett 27: 105-109.
Link M., Rausch T. and Greiner S. (2004). In Arabidopsis thaliana, the invertase inhibitors AtC/VIF1 and 2 exhibit distinct target enzyme specificities and expression profiles. FEBS Lett. 573, 105-109.
Miller E. M. and Chourey P. S. (1992). The maize invertase-deficient miniature-1 seed mutation is associated with aberrant pedicel and endosperm development. Plant Cell 2, 1107-1119.
Nitz I, Berkefeld H, Puzio P S, Grundler F M (2001) Pyk10, a seedling and root specific gene and promoter from Arabidopsis thaliana. Plant Sci 161: 337-346. Rausch T. and Greiner S. (2004). Plant protein inhibitors of invertases. Biochim. Biophys. Acta 1696, 253-261.
Roitsch T., Bittner M. and Godt D. E. (1995). Induction of apoplastic invertase of Chenopodium rubrum by D-glucose and a glucose analogue and tissue-specific expression suggest a role in sink-source regulation. Plant Physiol. 108, 285-294.
Roitsch T. and Gonzalez M.-C. (2004). Function and regulation of plant invertases: sweet sensations. Trends Plant Sci. 9, 606-613.
Siemens, J., Nagel, M., Ludwig-Müller, J., and Sacristan, M. D. 2002. The interaction of Plasmodiophora brassicae and Arabidopsis thaliana: Parameters for disease quantification and screening of mutant lines. J. Phytopath. 150:592-605.
Sturm A., Sebkova V., Lorenz K., Hardegger M., Lienhard S. and Unger C. (1995). Development- and organ-specific expression of the genes for sucrose synthase and three isoenzymes of acid β-fructosidase in carrot. Planta 195, 601-610.
Tang G.-Q., Lüscher M. and Sturm A. (1999). Antisense repression of vacuolar and cell wall invertase in transgenic carrot alters early plant development and sucrose partitioning. Plant Cell 11, 177-189.
Tymowska-Lalanne Z. and Kreis M. (1998). Expression of the Arabidopsis thaliana invertase gene family. Planta 207, 259-265.
Von Schweinichen and Büttner M. (2005). Expression of a plant cell wall invertase in roots of Arabidopsis leads to early flowering and an increase in whole plant biomass. Plant Biol. 7, 469-475.
Weber H., Borisjuk L., Heim U., Sauer N. and Wobus U. (1997). A role for sugar transporters during seed development: molecular characterization of a hexose and a sucrose carrier in fava bean seeds. Plant Cell 9, 895-908.
Weil M., Krausgrill S., Schuster A. and Rausch T. (1994). A 17-kDa Nicotiana tabacum cell-wall peptide acts as in-vitro inhibitor of the cell-wall isoform of acid invertase. Planta 193, 438-445.
Weschke W., Panitz R., Gubatz S., Wang Q., Radchuk R., Weber H. and Wobus U. (2003). The role of invertases and hexose transporters in controlling sugar ratios in maternal and filial tissues in barley caryopses during early development. Plant J. 33, 395-411.
Wobus U. and Weber H. (1999). Sugars as signal molecules in plant seed development. Biol. Chem. 380, 937-944.

The features of the present invention disclosed in the specification, the claims and/or the drawings may both separately and in any combination thereof be material for realizing the invention in various forms thereof.

1. A method for increasing shoot-to-root ratio of a plant comprising the step of inhibiting the activity of an invertase in the root tissue of said plant.

2. A method for increasing seed production of a plant comprising the step of inhibiting the activity of an invertase in the root tissue of said plant.

3. A method for increasing resistance to a disease of a plant comprising the step of inhibiting the activity of an invertase in the root tissue of said plant.

4. The method according to any of claims 1 to 3, wherein the activity of the invertase is inhibited either by
(d) a knock-down of the invertase or
(e) knock-out of the invertase, or
(f) an inhibitor to the invertase.

5. The method according to claim 4, wherein the inhibitor is active in the root tissue of the plant.

6. The method according to claim 4 or 5, wherein the inhibitor is a polypeptide.

7. The method according to any of claims 4 to 6, wherein the inhibitor is encoded by a nucleic acid.

8. The method according to claim 7, wherein the nucleic acid is under the control of a transcription element and/or a translation element, whereby such transcription element and/or such translation element allows for the specific transcription and/or translation of the nucleic acid in root tissue.

9. The method according to claim 8, whereby the transcription element is a promoter, preferably a root specific promoter.

10. The method according to claim 9, whereby the promoter is an inducible promoter.

11. The method according to claim 9 or 10, whereby the promoter is selected from the group comprising promoter pyk10, promoter T80-cryptic, and promoter WRKY6.

12. The method according to claim 11, whereby the promoter is promoter T80-cryptic.

13. The method according to any of claims 4 to 12, wherein the inhibitor is selected from the group comprising tobacco invertase inhibitors and Arabidopsis invertase inhibitors, whereby, preferably, the tobacco invertase inhibitors are selected from the group comprising NT-CIF1, Y12805; Nt-VIF, AY145781 and/or the Arabidopsis invertase inhibitors are selected from the group comprising AtC/VIF1, At1g47960; AtC/VIF2, At5g64620, and AtC/VIF3, At3g17130.

14. The method according to claim 4, wherein the knock-down is caused by post-transcriptional gene silencing and/or co-suppression.

15. The method according to any of claims 1 to 14, wherein the invertase is an invertase selected from the group comprising a soluble invertase, a vacuolar invertase, a neutral/alkaline invertase and a cytoplasmatic invertase.

16. The method according to any of claims 1 to 14, whereby the invertase is an invertase selected from the group comprising a cell wall bound invertase, and an extracellular, apoplasmic but not cell wall bound invertase, whereby preferably the invertase is a cell wall bound invertase.

17. The method according to claim 15, wherein the invertase is an invertase having an amino acid sequence, whereby the amino acid sequence is encoded by a nucleic acid which is selected from the group of nucleic acid sequences comprising nucleic acid sequences SEQ.ID.No. 1 to 14.

18. The method according to claim 16, wherein the invertase is an invertase having an amino acid sequence, whereby the amino acid sequence is encoded by a nucleic acid which is selected from the group of nucleic acid sequences comprising nucleic acid sequences SEQ.ID.No. 15 to 36.

19. The method according to any of claims 4 to 18 to the extent the claims refer to claim 3, wherein the disease of the plant is a disease involving or affecting the root tissue

20. The method according to any of claims 4 to 19 to the extent the claims refer to claim 3, wherein the disease of the plant is transferred or caused by a pathogen.

21. The method according to claim 20, wherein the pathogen is selected from the group comprising Plasmodiophora brassicacae, Verticillium and nematodes, whereby the nematode preferably is Heterodera schachtii Schm.

22. The method according to any of claims 19 to 21, wherein the disease is selected from the group comprising diseases which are caused by or associated with an organism selected from the group comprising Pythium aphanidermatum, Pythium ultimum, Phytophthora syringae P. undulata, Oxysporum f. sp. radicis-lycopersici, Meloidogyne hapla, Phytophtora quercina and Rhizoctonia solani Kuhn.

23. The method according to any of claims 1 to 22, wherein the plant is a member of the family of Brassicacae.

24. The method according to claim 23, wherein the plant is selected from the group comprising rapeseed, cabbage and china cabbage.

25. A nucleic acid molecule, preferably coding for an invertase, having a nucleic acid sequence, whereby be nucleic acid sequence is selected from the group of nucleic acid sequences SEQ.ID.No. 1 to 36, or a nucleic acid essentially complementary thereto.

26. A nucleic acid molecule which hybridizes, preferably under stringent conditions, to the nucleic acid sequence according to claim 25.

27. A nucleic acid molecule which, but for the degeneracy of the genetic code, would hybridize, preferably under stringent conditions, to the nucleic acid according to claim 25 or 26.

28. A polypeptide, preferably an invertase, encoded by a nucleic acid molecule according to any of claims 25 to 27.

29. A vector comprising a nucleic acid molecule according to any of claims 25 to 27.

30. The vector according to claim 29, whereby the vector is a plant vector, more preferable a plant expression vector.

31. The vector according to claim 30, wherein the vector comprises a root specific promoter.

32. A cell, preferably a plant cell, comprising nucleic acid molecule according to any of claims 25 to 27 and/or a vector according to any of claims 29 to 31.

33. A tissue and/or an organ comprising a nucleic acid molecule according to any of claims 25 to 27 and/or a vector according to any of claims 29 to 31 and/or a cell according to claim 32.

34. The tissue and/or organ according to claim 33, wherein the tissue is a root tissue and/or the organ is a root.

35. An organism, preferably a plant, comprising a nucleic acid molecule according to any of claims 25 to 27 and/or a vector according to any of claims 29 to 31 and/or a cell according to claim 32.

36. Use of a nucleic acid construct for the modification of the genome of a plant, whereby the construct comprises
(i) a root specific promoter; and
(j) a nucleic acid coding for an invertase inhibitor,
wherein the promoter and the nucleic acid coding for the invertase are operably linked to each other.

37. Use of a nucleic acid construct for inhibiting the activity or presence of an invertase, preferably an invertase in root and/or root tissue, whereby the construct comprises
(k) a root specific promoter; and
(l) a nucleic acid coding for an invertase inhibitor,
wherein the promoter and the nucleic acid coding for the invertase are operably linked to each other.

38. Use of a nucleic acid construct for increasing shoot-to-root ratio, seed production and/or resistance to disease of a plant, whereby the construct comprises
(m) a root specific promoter; and
(n) a nucleic acid coding for an invertase inhibitor,
wherein the promoter and the nucleic acid coding for the invertase are operably linked to each other.

39. Use of a nucleic acid construct for the manufacture of a medicament for the treatment of a plant disease, whereby the construct comprises
(o) a root specific promoter; and
(p) a nucleic acid coding for an invertase inhibitor,
wherein the promoter and the nucleic acid coding for the invertase are operably linked to each other.

40. Use according to claim 39, whereby the medicament is for gene therapy of a plant.

41. Use according to claim 40, whereby the plant is a plant cell or a plant tissue, preferably prior to regeneration to a mature plant.

42. Use of a nucleic acid construct for the generation of a transgenic plant, whereby the transgenic plant preferably shows one or more of an increase in shoot-to-root ratio, increase in seed production and increase in resistance to pathogens and/or diseases.

43. Use of a polypeptide according to claim 28 as a target molecule.

44. Use according to claim 43, wherein the polypeptide is a target molecule for an inhibitor in vitro and/or in vivo.

45. Use according to claim 43 or 44, wherein the target molecule is a target molecule in the root tissue of a plant.

46. Use according to claim 43, wherein the target molecule is used in s screening method for the identification of a plant protection agent.

47. Use according to claim 43, wherein the target molecule is used in s screening method for the identification of a plant growth promoter.


SEQUENCE LISTING (46 sequences)

SEQ ID NO: 1 - DNA, 407 bp, Brassica napus
gatccatcgactgcctggttcaacaaaaaagatgggtactggagaatgcttgtgggctcaaagaacaagcgtagaggaattgcttatatgtacaaaagtcgtgacttcaagaaatgggtcaaaactagacgtcccgtacacactagaaaagcaaccggtatgtgggaatgtcccgactttttcccggtttccattggcaagaaaaccggtttagacaccagctatgacggtccaaacaccaagcatgtgttgaaggttagcttggacctgaccaggtacgagtactacactcttggaacgtatgatactaagaaggatcgttacaagcctgatggtaccagtcctgatggttgggatggtttgagactagattatggtaacttctatgcgtcgaagtccttctttga

SEQ ID NO: 2 - DNA, 410 bp, Brassica napus
gaccccacaacggcctggcttggacgtgacggagaatggcgagtaatcgtcggaagctcgacggacgatcgacgaggattagcgattctttacaaaagcagagatttcttcaactggacgcaatcaacgaagcctttacattacgaagacttaaccggaatgtgggaatgtcctgattttttcccggtttcgataaccggatcggacggtgtagaagcgtcgtcggttggtgagaatgggattaagcatgtgcttaaagtgagtttgattgagacattgcatgtttaatacacgattgggagttatgatcgtgagaaagatgtttacgtaccggatcttgggtttgtgcaaaacgaatcaactccgaggttagattacgggaaatattacgcgtccaagtcgttcttcga

SEQ ID NO: 3 - DNA, 407 bp, Brassica napus
gatccgacctctgcttggttcaacaaaaaagatgggtactggagaatgcttgtgggctcaaagaacaagcgtagaggaattgcttatatgtacaaaagtcgtgacttcaagaaatgggtcaaaactagacgtcccgtacacactagaaaagcaaccggtatgtgggaatgtcccgactttttcccggtttccatcggcaagaaaaccggtttagacaccagctatgacggtccaaacaccaagcatgtgttgaaggttagcttggacctgaccaggtacgagtactacactcttggaacgtatgatactaagaaggatcgttacaagcctgatggtaccactcctgatggttgggatggtttgagactagattatggtaacttctatgcatcgaaaactttctacga

SEQ ID NO: 4 - DNA, 410 bp, Brassica napus
gacccgacgactgcttggaagacatcagccggaaaatggcggatcactattggttccaagatcaacagaaccgggatctcactcgtgtacgacacgactgacttcaagacttacgagaaactcgacacattgttgcacaaagttccaaacaccgggatgtgggagtgtgctgacttttatcctgtgtctaagaccttggttaaggggctcgactcatcggtcaatggaccagatgtgaagcacatcgtgaaggctagtatggacgacaccagaatcgaccattatgccataggaacatatttcgattcgaacgggacgtggatcccggatgatcctactattgatgttgggattagtactagtttaagatatgattacggaaagttctatgcgtcgaagacctttttcga

SEQ ID NO: 5 - DNA, 407 bp, Brassica napus
gaccccacgactgcttggttcaacaaaaaagatgggcactggagaatgcttgtgggctcaaagaacaagcgtagaggaattgcttatatgtacaaaagtcgtgacttcaagaaatgggtcaaaactagacgtcccgtacacactagaaaagcaaccggtatgtgggaatgtcccgactttttcccggtttccatcggcaagaaaaccggtttagacaccagctatgacggtccaaacaccaagcatgtgttgaaggttagcttggacctgaccaggtacgagtactacactcttggaacgtatgatactaagaaggatcgttacaagcctgatggtaccactcctgatggttgggatggtttgagactagattatggtaacttctatgcgtcgaagacctttttcga

SEQ ID NO: 6 - DNA, 407 bp, Brassica napus
gacccgaccacggcttggttcaacaaaaaagatgggcactggagaatgcttgtgggctcaaagaacaagcgtagaggaattgcttatatgtacaaaagtcgtgacttcaagaaatgggtcaaaactagacgtcccgtacacactagaaaagcaaccggtatgtgggaatgtcccgactttttcccggtttccatcggcaagaaaaccggtttagacaccagctatgacggtccaaacaccaagcatgtgttgaaggttagcttggacctgaccaggtacgagtactacactcttggaacgtatgatactaagagggatcgttacaagcctgatggtaccactcctgatggttgggatggtttgagactagattatggtaacttctatgcctcgaagacttttttcga

SEQ ID NO: 7 - DNA, 410 bp, Brassica napus
gatccaacaacggcttggttcaacaaggaagatgggtattggagaatgcttgttggctcaaagagaaagaacagaggaattgcttatatgtacaagagccgtgacttcaaaaaatgggtcaaaggcaaacatcctaaccactcaagaaagaaaaccggtatgtgggaatgtcccgatttcttcccggtattcgtaaccgacaagaaaaacggtttggacttcagctacgacggtccaaacgccaagcatgtgttgaaggttagtttggacttgaccagatacgagtactacactcttggaacgtatgacaccaagaaggatcgttacaggccagacggttacactcctgacggttgggatggtttgagatttgattatggtaactactatgcgtcaaaatcgttcttcga

SEQ ID NO: 8 - DNA, 412 bp, Brassica napus
gacccatccactgcttggctaggccaagacaagaaatggagagtgattatcggaagcaagattcaccgtcgtggactagccatcacttacacgagtaaagactttctaaaatggggaaaatctccagagccgtcgcattacgacgacggaagtggaatgtgggaatgtcctgattttttcccggtcacgaggtttggttctaacggcgtggaaacgtcttcgtttggtgaacctaatgagattttgaagcacgtgttgaaaataagtttggacgacacgaaacatgattataacacgattggtacgtacgatcgggttaaagataaattcgtaccggacaatggtttcaagatggacggtacggctccgagatacgatacggaaagtattacgcgtccaaaacattctatga

SEQ ID NO: 9 - DNA, 410 bp, Brassica napus
gatccgtccactgcctggttcaacaaaaaagatgggtatcggagaatgcttgttggctcaaagagaaagaacagaggaattgcttatatgtacaagagccgtgacttcagaaaatgggtcaaaagcaaacgtcctatccactcaagaaagaaaaccggtatgtgggaatgtcccgatttcttcccggtatccgtaaccgacaagaaaaacggtttggacttcagctacgacggtccaaacgccaagcatgtgttgaaggttagtttggacttgaccagatacgagtactacactcttggaacgtatgacaccaagaaggatcgttacaggccagacggttacactcctgacggttgggatggtttgagatttgattatggtaactactatgcgtcgaagtcattttacga

SEQ ID NO: 10 - DNA, 410 bp, Brassica napus
gacccaaccaccgcttggcttggacgtgacggagaatggcgagtaatcgtcggaagctcgacggacgatcgacgaggattagcgattctttacaaaagcagagatttcttcaactggacgcaatcaatgaagcctttacattacgaagacttaaccggaatgtgggaacgtcctgattttttcccggtttcgataaccggatcggacggtgtagaaacgtcgtcggttggtgagaatgggattaagcgtgtgcttaaagtgagtttgattgagacattgcatgattattacacgattgggagttatgatcgtgagaaagatgtttacgtaccggatcttgggtttgcgcaaaacgaatcagctccgaggttagattacgggaaatattacgcgtccaagtccttctacga

SEQ ID NO: 11 - DNA, 413 bp, Brassica napus
gatcccaccactgcctggctaggccaaggcaagaaatggagagtgatcatcggaagcaagattcaccgtcgtggactagccattacttacacgagtaaagactttctaaaatgggaaaaatctccagagccgttgcattacgacgacggaagtggaatgtgggaatgtcctgattttttcccagtcacgaggtttggttctaacggcgtggaaacgtcttcgtttggtgaacctaatgagattttgaagcacgtgttgaagataagtttggacgacacgaaacatgactattacacgattggtacgaacgatcgggtcaaagataaattcgtaccggacaatggtttcaagatggacggtacggttccgagatacgattatggaaagtattacgcgtcaaaaacgttcttcga

SEQ ID NO: 12 - DNA, 407 bp, Brassica napus
gacccgtcgacggcgtggtactccaaagacgggcattggagaaccgtggtagggtcaaaaagaaagcgtagaggaattgcttacatctacagaagccgagatttcaagcattgggtcaaagctaagcacccggttcactctaaacagtcaaccggtatgtgggaatgtcctgatttcttcccggtttccttaaccgatttccgaaacggtttggacttggattacgtcggtccaaacaccaagcatgtgttgaaggttagcttggacattacccggtacgagtattacacgcttggtaaatacgatcttaagaaggaccggtacataccggacggtaatactcccgatggttgggagggtttgagattcgattacggtaatttctacgcgtccaagtcatttttcga

SEQ ID NO: 13 - DNA, 408 bp, Brassica napus
gatccgtccactgcgtggcttggacgtgacggagaatggcgagtaatcgtcggaagctcgacggacgatcgacgaggattagcgattctttacaaaagcagagatttcttcaactggacgcaatcaacgaagcctttacatacgaagacttaaccggaatgtgggaatgtcctgattttttcccggtttcgataaccggatcggacggtgtagaaacgtcgtcggttggtgggaatgggattaagcatgtgctaaagtgagtttgattgggacattgcatgattattacacgattgggagttatgatcgtgagaaagatggttacgtaccggatcttgggtttgtgcaaaacgaatcagctccgaggttagattacgggaaatattacgcgtccaaatcattctacga

SEQ ID NO: 14 - DNA, 754 bp, Brassica napus
tccttcacctgttctaccagtacaatcccaaaggtgcggtttggggtaacattgtgtgggctcattcagtttctaaggacttgatcaattgggaagctcttgaaccggctatttacccctccaaatggtttgatatcaatggtacatggtccggttcagccaccaacgtaccgggaaaaggaccggttatcctctacactggtatcaccgagaaccatactcagatccaaaattatgccattccccaagacctttccgacccatacctcaagaaatggatcaagcccgacgacaaccctatcgtaagacccgaccatggcgagaatggatccgctttccgtgacccgacaactgcttggttcaacaaaaaagatgggcactggagaatgcttgtgggctcaaagaacaagcgtagaggaattgcttatatgtacaaaagtcgtgacttcaagaaatgggtcaaaactagacgtcccgtacacactagaaaagcaaccggtatgtgggaatgtcccgactttttcccggtttccatcggcaagaaaaccggtttagacaccagctatgacggtccaaacaccaagcatgtgttgaaggttagcttggacctgaccaggtacgagtactacactcttggaacgtatgatactaagaaggatcgttacaagcctgatggtaccactcctgatggttgggatggtttgagactagattatggtaacttctacgcatcaaaaactttctacgaaaggaa

SEQ ID NO: 15 - DNA, 759 bp, Brassica napus
tccttcacttgttttatcagtacaatccatacggcgccgtttgggatgtaagaatcgtgtggggtcactccacgtcacttgatctagttaactggaccccacagcctccagcattcagtccatctcagccgtcagacatcaacggttgttggtcaggctccgtcacgattctaccaaacggcacaccggtgatcctctacaccggcattgaccaaaacaaaagtcaagtccaaaacgtcgccgttccgcttaacatctccgatccatatctccgcgaatggtcaaagtcgccggcaaatcctctgatggctcctaacgccgtcaacggaatcaaccccgaccggttccgagacccgaccaccgcgtggctaggacacgacggagaatggagagtcatcgtcggaagctcgacggacgatcgtcgaggattagcggttctttacaagagcagagatttcttcaactggacgcaagcgacgaagcctcttcaccacgaagacttaaccggaatgtgggagtgtcctgattttttcccggtttcgataaccggaacggacggtctcgagacgtcgtcgtttggtgaggtgaaacacgtgctgaaagtgagtttgatcgagacgttgcatgattattacacggttgggagttacgaccgtgagaaggatgtttacgtaccggatcatgggtttgtgcaagatggttcggctccgaggctggactacgggaagttttacgcaaccaaaaccttctacgaaagga

SEQ ID NO: 16 - DNA, 759 bp, Brassica napus
tccttcacttgttttaccagtataatccatacggcgccgtttgggatgtaagaatcgtgtggggtcactccacgtcacttgatctagttaactggaccccacagcctccagcattcagtccatctcagccgtcagacatcaacggttgttggtcaggctccgtcacgattctaccaaacggcacaccggtgatcctctacaccggcattgaccaaaacaaaagtcaagtccaaaacgtcgccgttccgcttaacatctccgatccatatctccgcgaatggtcaaagtcgccggcaaatcctctgatggctcctaacgccgtcaacggaatcaaccccgaccggttccgagacccgaccaccgcgtggctaggacacgacggagaatggagagtcatcgtcggaagctcgacggacgatcgtcgaggattagcggttctttacaagagcagagatttcttcaactggacgcaagcgacgaagcctcttcaccacgaagacttaaccggaatgtgggagtgtcctgattttttcccggtttcgataaccggaacggacggtctcgagacgtcgtcgtttggtgaggtgaaacacgtgctgaaagtgagtttgatcgagacgttgcatgattattacacggttgggagttacgaccgtgagaaggatgtttacgtaccggatcatgggtttgtgcaagatggttcggctccgaggctggactacgggaagttttacgcatccaagaccttttatgaaagga

SEQ ID NO: 17 - DNA, 759 bp, Brassica napus
tccttcacttcttttatcagtataatccatacggcgccgtttgggatgtaagaatcgtgtggggtcactccacgtcacttgatctagttaactggaccccacagcctccagcattcagtccatctcagccgtcagacatcaacggttgttggtcaggctccgtcacgattctaccaaacggcacaccggtgatcctctacaccggcattgaccaaaacaaaagtcaagtccaaaacgtcgccgttccgctcaacatctccgatccatatctccgcgaatggtcaaagtcgccggcaaatcctctgatggctcctaacgccgtcaacggaatcaaccccgaccggttccgagacccgaccaccgcgtggctaggacacgacggagaatggagagtcatcgtcggaagctcgacggacgatcgtcgaggattagcggttctttacaagagcagagatttcttcaactggacgcaagcgacgaagcctcttcaccacgaagacttaaccggaatgtgggagtgtcctgattttttcccggtttcgataaccggaacggacggtctcgagacgtcgtcgtttggtgaggtgaaacacgtgctgaaagtgagtttgatcgagacgttgcatgattattacacggttgggagttacgaccgtgagaaggatgtttacgtaccggatcatgggtttgtgcaagatggttcggctccgaggctggactacgggaagttttacgcatccaaaaccttctacgaaagga

SEQ ID NO: 18 - DNA, 753 bp, Brassica napus
tccttcacttcttttaccagtataacccttacgatgcacaatccggaaacatagtctggggacattctacatcaactgatcttatcaactggacacctcagccgccggcactactccgatcagagccttatgattttaaaggctgtttttcaggttctacaacaattctctccggcggaaaaccggcaattctctataccggagtagacttctccgatatccaagttcaaaatctagccgtgcccaaaaatttattggacccttaccttatagaatgggtaaaatcaccttataatccactaataacacctaattcagtgaataaaattgatggtcaaaatttcagagacccgactactgcttgggtaaatcctacagatggtaattggagaatggtagttggaaataaaaaaaataacacaggaattggtttattgtacaaaagcaagaattttattgattggattcaaactgaacaacctttgcattttttaaacaattctggaatgtgggaatgtcctgattttttcccagtttcaacaattagtcaaattggtttagacacttcgattatgggtccaaatgtaaaacatgtattcaaagtaagtgtagcaaattctgattactatacaattggaatatataatccaaataaggatatctttgtcccggataatgaatccttggatattggattaggatttagatatgattatggaaagtattacgcatcaaaaaccttctttgaaagga

SEQ ID NO: 19 - DNA, 702 bp, Brassica napus
tccttcacctgttttatcagtataacccttacgatgcacaatccggaaacatagtctggggacattctacatcaactgatcttatcaactggacacctcagccgccggcactactccgatcagagccgtatgattttaaaggctgtttttcaggttctacaacaattctctccggcggaaaaccggcaattctctataccggagtagacttctccgatatccaagttcaaaatctagccgtgcccaaaaatttattggacccttaccttatagaatgggtaaaatcaccttataatccactaataacacctaattcagtgaataaaattgatggtcaaaatttcagagacccaactactgcttgggtaaatcctacagatggtaattggagaatggtagttggaaataaaaaaaataacacaggaattggtttattgtacaaaagcaagaattttattgattggattcaaactgaacaacctttgcattttttaaacaattctggaatgtgggaatgtcctgattttttcccagtttcaacaattagtcaaattggtttagacacttcaattatgggtccaaatgtaaaacatgtattcaaagtaagtgtagcaaattctgattactatacaattggaatatataatccaaataaggatatttttgtcccggataatgaatccttggatattggattaggat

SEQ ID NO: 20 - DNA, 752 bp, Brassica napus
tccttcacttgttttatcagtacaacccttacgatgcacaatccggaaacatagtctggggacattctacatcaactgatcttatcaactggacacctcagccgccggcactactccgatcagagccttatgattttaaaggctgtttttcaggttctacaacaattctctccggcggaaaaccggcaattctctataccggagtagacttctccgatatccaagttcaaaatctagccgtgcccaaaaatttattggacccttaccttatagaatgggtaaaatcaccttataatccactaataacacctaattcagtgaataaaattgatggtcaaaatttcagagacccaactactgcttgggtaaatcctacagatggtaattggagaatggtagttggaaataaaaaaaataacacaggaattggtttattgtacaaaagcaagaattttattgattggattcaaactgaacaacctttgcattttttaaacaattctggaatgtgggaatgtcctgattttttcccagtttcaacaattagtcaaattggtttagacacttcaattatgggtccaaatgtaaaacatgtattcaaagtaagtgtagcaaattctgattactatacaattggaatatataatccaaataaggatatttttgtcccggataatgaatccttggatattggattaggatttagatatgattatggaaagtattacgctaccaaaccttctacgaaagga

SEQ ID NO: 21 - DNA, 752 bp, Brassica napus
tccttcactttttctatcagtacaatccttacgatgcacaatccggaaacatagtctggggacattctacatcaactgatcttatcaactggacacctcagccgccgcactactccgatcagagccttatgattttaaaggctgtttttcaggttctacaacaattctctccggcggaaaaccggcaattctctataccggagtagacttctccgatatccaagttcaaaatctagccgtgcccaaaaatttattggacccttaccttatagaatgggtaaaatcaccttataatccactaataacacctaattcagtgaataaaattgatggtcaaaatttcagagacccaactactgcttgggtaaatcctacagatggtaattggagaatggtagttggaaataaaaaaaataacacaggaattggtttattgtacaaaagcaagaattttattgattggattcaaactgaacaacctttgcattttttaaacaattctggaatgtgggaatgtcctgattttttcccagtttcaacaattagtcaaattggtctagacacttcaattatgggtccaaatgtaaaacatgtattcaaagtaagtgtagcaaattctgattactatacaattggaatatataatccaaataaggatatttttgtcccggataatgaatccttggatattggattaggatttagatatgattatggaaagtattacgcatccaaaaccttttacgaaagga

SEQ ID NO: 22 - DNA, 752 bp, Brassica napus; identical to SEQ ID NO: 20

SEQ ID NO: 23 - DNA, 410 bp, Brassica napus
gatccctcgactgcatggaagacatcagccggaaaatggcggatcactattggttccaaaatcaacagaaccgggatctcactcgtgtacgacacgactgacttcaagacttacgagaaactcgacacattgttgcacaaagttccaaacaccgggatgtgggagtgtgttgacttttatccagtgtctaagaccttggttaaggggctcgacacatcggtcaatggaccagatgtgaagcacgtcgtgaaggctagtatggacgacaccagaatcgaccattatgccataggaacatatttcgattcgaacgggacgtggatcccggatgatcctactattgatgttgggattagtactagtttaagatatgattgcggaaagttttatgcctccaagtccttttacga

SEQ ID NO: 24 - DNA, 444 bp, Brassica napus
gacccatcgacggcctggaagacatcagatggaaaatggcggatcacaatcggttccaagatcaacaaaaccgggatctcactagtgtacgacacaatcgacttcaagacttacgagaaacacgacacattgttgcacaaggttccaaacacggggatgtgggagtgtgttgacttctatccagtgtctaagaccgcgatcaatgggctcgacacatcagtcaacggaccaaatgtgaagcacatcgtgaaggctagcatggacgacaccaggtttgatcattatgccgtggggacgtactttgattcgaacggaacatggatcccggatgatcctactattgatgttgggatgagtgctagtttgagatatgattacggaaagttctatgcctcaaagtcattctatgaatggtaactactatgcgtcgaagtcattttacga

SEQ ID NO: 25 - DNA, 782 bp, Brassica napus
tccttcacttgttttacaatataatcctttagctccagagttcagtagaagaatcatatggggccactctgtttcacaagacatggtcaactggatccaactcccgccagcactttctccctctgagtcctacgacatcaacagctgctggtcaggatccgctacgatcctccccgatggcaaacctgtgatcttgtacaccggaatcgataaccaagagagacgggaagacagacggcaagtcacagttcttgccgtacctaaagatgcttccgaccctttgcttcgtgagtggatgaagccaaagcaaaaccctgtcatggatccatcagaggacatccttcactactgtttccgtgaccctaccactgcatggcaaggtcaagatggtaaatggagagttctcataggagctaaggagagagatactctaagaggagtagctcttttgtaccatagtactgatgattgtgagcaatggactaggtatcaagaacctttacttgtagcacaagccaacgaaatgttggagtgcgttgactttttcccggttaagctcatgggtaaagaaggtgtagatacttcggtgaacaatgctagtgtgaggcatgtgttgaaagttagttttgaggaagaacttggaggcaaagattgttatgttattggctcatattgttccgagactgatagatttgtcccggactcagagctcacttacacacgtgctgatctgagatatgatgatggatggttttacgctaccaaaaccttctatgaaagga

SEQ ID NO: 26 - DNA, 952 bp, Brassica napus
tccttcactttttctatcaatataacccaaatgcagccgtgtggggtgacattgtttggggtcacgcagtgtctaaagatcttatccattggcttcacctcccgtttgcaatggttcctgaccaatggtacgacgctaacggtgtttggaccggttcggctactttcctcgatgatggttctattgtcatgctctacactggatccaccgacaaattcgtacaagtattactttttaaaaccatttctctgttaaataagtatttgattcataattcttttgatatgatatcaataactcttttgatatactggtgacagatttatataaaaccttaattaatcctttaaaaatcatttcaaaataagatttatcggaccaattagcgataataatattagctattattatgtttttaggttcaaaaccttgccctatcctgaggaccccaatgatccacttttgttgaaatggaccaagttctctggcaacccggttctcgaaccgcctccaggtatcggtgataaggacttccgtgacccaacaactgcgtggaagacatcagacggaaaatggcggatcactatcggccccaaaatcaatagaaccggaatatcccttgtttatgacaccgtcgatttcaagacctacgagaaacacgacatcttgttgcaccaagtcccaaacacaggaatgtgggagtgcgttgacttttatccggtgtcaaagactaagcaccacggtcttgacacttcagttaatggaccggatgttaggcatatagtgaaagctagcatggacgatacaagaattgaccattatgccattgggacgtactatgattctaatggaacatgggtcccggataatccttcaattgatgttgggattagtaccggtttgagatacgattacggtaaattttacgcaacaaaaactttctacgaaagga

SEQ ID NO: 27 - DNA, 719 bp, Brassica napus
tccttcacttattctatcaatataatccaaatgcagccgtgtggggtgacattgtttggggtcacgctgtatcaaaggatcttatccactggctttacctcccatttgccatggttcctgaccaatggtacgatgctaacggcgtttggaccggttcagctactttccttgatgatggttctattgtcatgctctacactggttccaccgacaaattcgtacaggtaatgatttttaaaatccatttattgcttcattaatcactttagtaatgcttgtgacagtataaaacgtgaataattgtttcataattaatattgattccaattcttgtgttaatgttgttatttactacgcttttaggttcaaaaccttgcctatcctgaagacccaaaagatccacttttgttgaaatggaccaagttctccggcaacccggttcttgtaccacctccaggtatcggtgctaaggacttccgtgacccaacaactgcatggaagacttcagacgggaaatggcggatcactattggctccaaaatcaacagaaccggaatatctcttatttacgatacaacagatttcaagacctacgagaaacatgagaccttgttgcaccaagtcccaaacactggagtgggagtgtgttgacttttatccagtgtccaagactaaagacaagggtcttgacacttcggtcaatggaccggatgttaagc

SEQ ID NO: 28 - DNA, 945 bp, Brassica napus
tccttcacttgttttaccagtacaatccaaatggagccgtgtggggtgacattttttggggtcacgctgtatcaaaggatcttatccactggctttacctcccatttgccatggttcctgaccagtggtacgatgctaacggcgtttggaccggttcggctactttccttgatgatggttctattgtcatgctctacactggttccaccgacaaatttgtacaagtaatgattttgaaaatccatttgttgcttaactaaccactctattaatgcttgtgacagtatggaatgtgaataaatgtttcataaatgtaatagtgattccaattcttgtgtttggtagcataaaatatatttcaatataaatgtttaaagcaattagcgacaatatgttgtttactacgcttttaggttcaaaaccttgcctatcctgaagaccctaaagatccacttttgttgaaatggaccaagttctccggcaacccggttcttgtaccacctccaggtatcggtgctaaggacttccgtgacccaacaactgcatggaagacatcagacgggaaatggcggatcactattggctccaaaatcaacagaaccggaatatctcttatttacgatacaacagatttcaagacctacgagaagcatgagaccttgttgcaccaagtcccaaacactggaatgtgggagtgcgttgacttttatccagtgtccaagactaaagacaagggtcttgacacttcggtcaatggaccggatgttaagcatatcattaaggctagcatggacgatactagaattgaccattatgccattgggacatactatgactctaatgcaacatgggtccccgataatccttcaatcgatgtcgggattagtaccggtttgagatatgattacggtaaattttacgcaaccaagaccttcttcgaaagga

SEQ ID NO: 29 - DNA, 948 bp, Brassica napus
tccttcacttgttttatcaatataacccaaatgcagccgtgtggggtgacattgtttggggtcacagtgtctaaagatcttatccattggcttcacctcccgtttgcaatggttcctgaccaatggtacgacgctaacggtgtttggaccggttcggctactttcctcgatgatggttctattgtcatgctctacactggatccaccgacaaattcgtacaagtattacttttaaaaccatttctctgttaaataagtatttgattcataatgcttttgatatgatatcaataaatcttttgatatactggtgacagatttatataaaacgtaattaattctttataaatcgtttcatataagattttatcggaccaattaacgataataatattagctattattatgtttttaggttcaaaaccttgcctatcctgaggaccccaatgatccacttttgttgaaatggaccaagttctctggcaacccggttctcgaaccgcctccaggtatcggtgataaggacttccgtgacccaacaactgcgtggaagacatcagacggaaaatggcggatcactatcggtctccaaaatcaatagaaccggaatatcccttgtttatgacacagtcgatttcaagacctacgagaaacacgatatcttgttgcaccaagtcccaaacactggaatgtgggagtgcgttgacttttatccggtgtcaaagactaagcaccacggtcttgacacttcagttaatggaccggatgttaggcatatagtgaaagctagcatggacgatacaagaattgaccattttgccattgggacgtactatgattctaatggaacatgggtcccggataatccttcaattgatgtcgggattagtaccggtttgagatacgattacggtaaattttacgcatccaaaaccttctacgaaagga

SEQ ID NO: 30 - DNA, 894 bp, Brassica napus
tccttcacctgttttatcagtacaacccaaatgcagccgtgtggggtgacattgtttggggtcacgctgtatcaaaggatcttatccactggctttacctcccatttgccatggttcctgaccaatggtacgatgctaacggcgtttggaccggttcagctactttccttgatgatggttctattgtcatgctctacactggttccaccgacaaattcgtacaggtaatgatttttaaaatccatttattgcttcattaatcactttagtaatgcttgtgacagtataaaacgtgaataattgtttcataattaatattgattccaattcttgtgttaatgttgttatttactacgcttttaggttcaaaaccttgcctatcctgaagacccaaaagatccacttttgttgaaatggaccaagttctccggcaacccggttcttgtaccacctccaggtatcggtgctaaggacttccgtgacccaacaactgcatggaagacttcagacgggaaatggcggatcactattggctccaaaatcaacagaaccggaatatctcttatttacgatacaacagatttcaagacctacgagaaacatgagaccttgttgcaccaagtcccaaacactggaatgtgggagtgtgttgacttttatccagtgtccaagactaaagacaagggtcttgacacttcggtcaatggaccggatgttaagcatatcattaaggctagcatggacgatactagaattgaccattatgccattgggacatactatgactctaatgcaacatgggtccccgacaatccttcaatcgatgtcgggattagtaccggtttgagatatgattacggtaaattttacgctccaagaccttctttgaaagga

SEQ ID NO: 31 - DNA, 939 bp, Brassica napus
tccttcactttttctaccagtataacccaaatgcagccgtgtggggtgacattgtttggggtcacgcagtgtctaaagatcttatccattggcttcacctcccgtttgcaatggttcctgaccaatggtacgacgctaacggtgtttggaccggttcggctactttcctcgatgatggttctattgtcatgctctacactggatccaccgacaaattcgtacaagtattactttttaaaaccatttctctgttaaataagtatttgattcataattcttttgatatgatatcaataactcttttgatatactggtgacagatttatataaaacttaattaattctttaaaaatcatttcaaaataagattttatcggaccaattagcgataataatattagctattattatgtttttaggttcaaaaccttgcctatcctgaggaccccaatgatccacttttgttgaaatggaccaagttctctggcaacccggttctcgaaccgcctccaggtatcggtgataaggacttccgtgacccaacaactgcgtggaagacatcagacggaaaatggcggatcactatcggctccaaaatcaatagaaccggaatatcccttgtttatgacaccgtcgatttcaagacctacgagaaacacgacatcttgttgcaccaagtcccaaacacaggaatgtgggagtgcgttgacttttatccggtgtcaaagactaagcaccacggtcttgacacttcagttaatggaccggatgttaggcatatagtgaaagctagcatggacgatacaagaattgaccattatgccattgggacgtactatgattctaatggaacgtgggtcccggataatccttcaattgatgttgggattagtaccggtttgagatacgattacggtaaattttacgcatcaaaaacca

SEQ ID NO: 32 - DNA, 710 bp, Brassica napus
tccttcactttttttatcagtacaaccctgattcagctatttggggaaatatcacatggggccatgcaatatccacggacttgatccattggctttacttgcccttcgccttggttcctgatcaatggtacgatatcaacggtgtctggaccgggtccgcgaccttcctacccgacggtcagatcatgatgttatacaccggtgataccaatgattacgtgcaggtgcaaaatcttgcgtaccccgccaacttatcggatcccctcctcatcgactgggtcaagtaccggggcaacccggtcatggttccaccacccggcattggtgtcaaggactttagagacccaacgactgcttggaccggaccacaaaacgggcagtggctgcttaccatcgggtccaagattggtaaaacgggtattgcaattgtttatggtacttccaacttcacaaactttaagctattggatggagttttgcatgcggttccgggtacgggtatgtgggagtgtgtggacttttacccggtatcaaccgatgaggcaaacgggttggacacatcatataacgggccaggtataaagcatgtgttaaaagcaagtttagatgacgataagcatgattactatgctattgggacatatgacccggtaaagaacaaatggactcctgataacccgcaattggatgtgggtatcgggttgagacta

SEQ ID NO: 33 - DNA, 753 bp, Brassica napus
tccttcacttgttttatcagtacaaccctgattcagctatttggggaaatatcacatggggccatgcaatatccacggacttgatccattggctttacttgcccttcgccttggttcctgatcaatggtacgatatcaacggtgtctggaccgggtccgcgaccttcctacccgacggtcagatcatgatgttatacaccggtgataccaatgattacgtgcaggtgcaaaatcttgcgtaccccgccaacttatcggatcccctcctcatcgactgggtcaagtaccggggcaacccggtcatggttccaccacccggcattggtgtcaaggactttagagacccaacgactgcttggaccggaccacaaaacgggcagtggctgcttaccatcgggtccaagattggtaaaacgggtattgcaattgtttatggtacttccaacttcacaaactttaagctattggatggagttttgcatgcggttccgggtacgggtatgtgggagtgtgtggacttttacccggtatcaaccgatgaggcaaacgggttggacacatcatataacgggccaggtataaagcatgtgttaaaagcaagtttagatgacgataagcatgattactatgctattgggacatatgacccggtaaagaacaaatggactcctgataacccgcaattggatgtgggtatcgggttgagactggactacgggaaatactacgcaaccaaaaccttcttcgaaagga

SEQ ID NO: 34 - DNA, 753 bp, Brassica napus
tccttcacttgttttaccagtataaccctgattcagctatttggggaaatatcacatggggccatgcaatatccacggacttgatccattggctttacttgcccttcgccttggttcctgatcaatggtacgatatcaacggtgtctggaccgggtccgcgaccttcctacccgacggtcagatcatgatgttatacaccggtgataccaatgattacgtgcaggtgcaaaatcttgcgtaccccgccaacttatcggatcccctcctcatcgactgggtcaagtaccggggcaacccggtcatggttccaccacccggcattggtgtcaaggactttagagacccaacgactgcttggaccggaccacaaaacgggcagtggctgcttaccatcgggtccaagattggtaaaacgggtattgcaattgtttatggtacttccaacttcacaaactttaagctattggatggagttttgcatgcggttccgggtacgggtatgtgggagtgtgtggacttttacccggtatcaaccgatgaggcaaacgggttggacacatcatataacgggccaggtataaagcatgtgttaaaagcaagtttagatgacgataagcatgattactatgctattgggacatatgacccggtaaagaacaaatggactcctgataacccgcaattggatgtgggtatcgggttgagactggactacgggaaatactacgcatccaaaaccttcttcgaaagga

SEQ ID NO: 35 - DNA, 753 bp, Brassica napus
tccttcacttgttctatcagtataaccctgattcagctatttggggaaatatcacatggggccatgcaatatccacggacttgatccattggctttacttgcccttcgccttggttcctgatcaatggtacgatatcaacggtgtctggaccgggtccgcgaccttcctacccgacggtcagatcatgatgttatacaccggtgataccaatgattacgtgcaggtgcaaaatcttgcgtaccccgccaacttatcggatcccctcctcatcgactgggtcaagtaccggggcaacccggtcatggttccaccacccggcattggtgtcaaggactttagagacccaacgactgcttggaccggaccacaaaacgggcagtggctgcttaccatcgggtccaagattggtaaaacgggtattgcaattgtttatggtacttccaacttcacaaactttaagctattggatggagttttgcatgcggttccgggtacgggtatgtgggagtgtgtggacttttacccggtatcaaccgatgaggcaaacgggttggacacatcatataacgggccaggtataaagcatgtgttaaaagcaagtttagatgacgataagcatgattactatgctattgggacatatgacccggtaaagaacaaatggactcctgataacccgcaattggatgtgggtatcgggttgagactggactacgggaaatactacgcttccaaaaccttctacgaaagga

SEQ ID NO: 36 - DNA, 753 bp, Brassica napus
tccttcacttgttctatcagtataaccctgattcagctatttggggaaatatcacatggggccatgcaatatccacggacttgatccattggctttacttgcccttcgccttggttcctgatcaatggtacgatatcaacggtgtctggaccgggtccgcgaccttcctacccgacggtcagatcatgatgttatacaccggtgataccaatgattacgtgcaggtgcaaaatcttgcgtaccccgccaacttatcggatcccctcctcatcgactgggtcaagtaccggggcaacccggtcatggttccaccacccggcattggtgtcaaggactttagagacccaacgactgcttggaccggaccacaaaacgggcagtggctgcttaccatcgggtccaagattggtaaaacgggtattgcaattgtttatggtacttccaacttcacaaactttaagctattggatggagttttgcatgcggttccgggtacgggtatgtgggagtgtgtggacttttacccggtatcaaccgatgaggcaaacgggttggacacatcatataacgggccaggtataaagcatgtgttaaaagcaagtttagatgacgataagcatgattactatgctattgggacatatgacccggtaaagaacaaatggactcctgataacccgcaattggatgtgggtatcgggttgagactggactacgggaaatactacgcttccaaaaccttcttcgaaagga

SEQ ID NO: 37 - DNA, 34 bp, artificial sequence
  primer
ctgaggtacctcgagcctgaaatggcttcttctc

SEQ ID NO: 38 - DNA, 34 bp, artificial sequence
  primer
ctgatctagagggccctcattcaacaaggcgatc

SEQ ID NO: 39 - DNA, 35 bp, artificial sequence
  primer
ctgaggtaccctcgagaagatgaagatgatgaagg

SEQ ID NO: 40 - DNA, 34 bp, artificial sequence
  primer
gatctctagagggccctcaaagcaacattctcac

SEQ ID NO: 41 - DNA, 22 bp, artificial sequence
  primer
gatgtacacgttttggtgtggg

SEQ ID NO: 42 - DNA, 22 bp, artificial sequence
  primer
gcttacgtgtttagggaaatgg

SEQ ID NO: 43 - DNA, 27 bp, artificial sequence
  primer
ggacggtaccctgcaacgaagtgtacc

SEQ ID NO: 44 - DNA, 30 bp, artificial sequence
  primer
gcaggtaccgtaattctgattttattcaag

SEQ ID NO: 45 - DNA, 33 bp, artificial sequence
  primer
gatcggtacctcgaattgtgatatattgtaagc

SEQ ID NO: 46 - DNA, 33 bp, artificial sequence
  primer
catggggtaccctgattaattagcaattagtgg